(19) United States
(12) Patent Application Publication
(10) Pub. No.: US 2015/0336985 A1
(43) Pub. Date: Nov. 26, 2015

(54) FUNGICIDAL HETEROCYCLIC COMPOUNDS

(71) Applicant: E. I. DU PONT DE NEMOURS AND COMPANY, Wilmington, DE (US)

(72) Inventors: Mei H. Dung, Garnet Valley, PA (US); Robert James Pasteris, Newark, DE (US)

(21) Appl. No.: 14/410,302

(22) PCT Filed: May 31, 2013
(86) PCT No.: PCT/US13/43517

§ 371 Date: Dec. 22, 2014

Related U.S. Application Data
(60) Provisional application No. 61/663,161, filed on Jun. 22, 2012.

(51) Int. Cl. C07D 513/04; A01N 43/90; A01N 43/56; C07D 417/06; A01N 43/60

(52) U.S. Cl. CPC C07D 513/04; C07D 417/06; A01N 43/60; A01N 43/56; A01N 43/90

(57) ABSTRACT

Disclosed are compounds of Formula 1, including all geometric and stereoisomers, tautomers, N-oxides, and salts thereof,

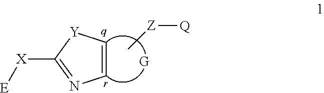

wherein
E, X, Y, G, Z and Q are as defined in the disclosure.
   Also disclosed are compositions containing the compounds of Formula 1 and methods for controlling plant disease caused by a fungal pathogen comprising applying an effective amount of a compound or a composition of the invention.

### FIELD OF THE INVENTION

This invention relates to certain heterocyclic compounds, their tautomers, N-oxides, salts and compositions, and methods of their use as fungicides.

### BACKGROUND OF THE INVENTION

The control of plant diseases caused by fungal plant pathogens is extremely important in achieving high crop efficiency. Plant disease damage to ornamental, vegetable, field, cereal, and fruit crops can cause significant reduction in productivity and thereby result in increased costs to the consumer. Many products are commercially available for these purposes, but the need continues for new compounds which are more effective, less costly, less toxic, environmentally safer or have different sites of action.

PCT Patent Publications WO 2007/014290, WO 2008/013925, WO 2008/091580 and WO 2011/085170 disclose amide fungicides.

### SUMMARY OF THE INVENTION

This invention is directed to compounds of Formula 1 (including all geometric and stereoisomers), tautomers, N-oxides, and salts thereof, agricultural compositions containing them and their use as fungicides:

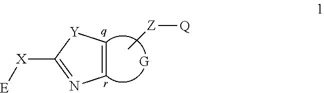

wherein
E is a radical selected from the group consisting of

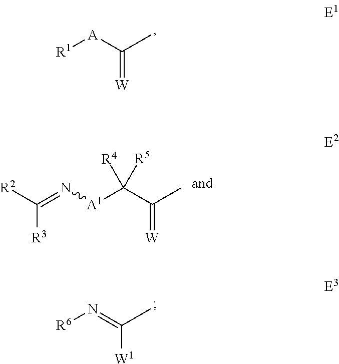

X is a radical selected from the group consisting of

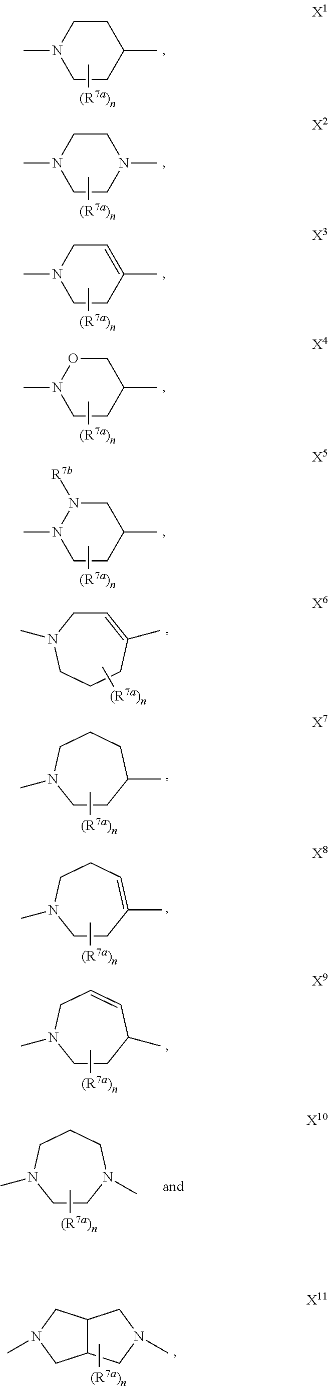

wherein the bond projecting to the left is connected to E, and the bond projecting to the right is connected to the carbon atom in Formula 1;
Y is O, S, NH or N(CH₃);
G together with the two carbon atoms indentified as “q” and “r” in Formula 1 forms a 5- to 6-membered ring containing ring members selected from carbon atoms and up to 2 heteroatoms independently selected from up to 1 O, up to 1 S and up to 2 N atoms, wherein up to 1 carbon atom ring member is selected from C(═O), C(═S) and C(═NOH), the ring optionally substituted with up to 2 substituents independently selected from R⁸ on carbon atom ring members and methyl on nitrogen atom ring members;
Z is a saturated, partially unsaturated or fully unsaturated chain containing 1- to 3-atoms selected from up to 3 carbon, up to 1 O, up to 1 S and up to 2 N atoms, the chain optionally substituted with up to 2 substituents independently selected from R^{9a} on carbon atoms and R^{9b} on nitrogen atoms;
Q is phenyl or naphthalenyl, each optionally substituted with up to 3 substituents independently selected from R^{10a}; or
a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, each ring or ring system optionally substituted with up to 3 substituents independently selected from R^{10a} on carbon atom ring members and R^{10b} on nitrogen atom ring members; or
a 3- to 7-membered nonaromatic carbocyclic ring, a 5- to 7-membered nonaromatic heterocyclic ring or an 8- to 11-membered nonaromatic bicyclic ring system, each ring or ring system containing ring members selected from carbon atoms and up to 4 heteroatoms independently selected from up to 2 O, up to 2 S and up to 4 N atoms, wherein up to 3 carbon atom ring members are independently selected from C(═O) and C(═S), and the sulfur atom ring members are independently selected from S(═O)ₛ(═NR²⁰)_{f}, each ring or ring system optionally substituted with up to 3 substituents independently selected from R^{10a} on carbon atom ring members and R^{10b} on nitrogen atom ring members;
A is CH(R¹¹), N(R¹²) or C(═O);
A¹ is O, S, C(R¹⁴)₂, N(R¹³), —OC(R¹⁴)₂—, —SC(R¹⁴)₂— or —N(R¹³)C(R¹⁴)₂—, wherein the bond projecting to the left is connected to the nitrogen atom, and the bond projecting to the right is connected to the carbon atom in Formula 1;
W is O or S;
W¹ is OR¹⁵, SR¹⁶, NR¹⁷R¹⁸ or R¹⁹;
R¹ and R⁶ are each optionally substituted phenyl, optionally substituted naphthalenyl or an optionally substituted 5- to 6-membered heteroaromatic ring; or cyano, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₅-C₁₀ alkylcycloalkylalkyl, C₂-C₈ alkoxyalkyl, C₂-C₈ haloalkoxyalkyl, C₄-C₁₀ cycloalkoxyalkyl, C₃-C₁₀ alkoxyalkoxyalkyl, C₂-C₈ alkylthioalkyl, C₂-C₈ haloalkylthioalkyl, C₂-C₈ alkylsulfinylalkyl, C₂-C₈ alkylsulfonylalkyl, C₂-C₈ alkylaminoalkyl, C₂-C₈ haloalkylaminoalkyl, C₃-C₁₀ dialkylaminoalkyl, C₄-C₁₀ cycloalkylaminoalkyl, C₃-C₈ alkoxycarbonylalkyl, C₃-C₈ haloalkoxycarbonylalkyl, C₁-C₈ alkoxy, C₁-C₈ haloalkoxy, C₂-C₈ alkenyloxy, C₂-C₈ haloalkenyloxy, C₂-C₈ alkynyloxy, C₃-C₈ haloalkynyloxy, C₃-C₈ cycloalkoxy, C₃-C₈ halocycloalkoxy, C₄-C₁₀ cycloalkylalkoxy, C₂-C₈ alkoxyalkoxy, C₂-C₈ alkylcarbonyloxy, C₂-C₈ haloalkylcarbonyloxy, C₁-C₈ alkylthio, C₁-C₈ haloalkylthio, C₃-C₈ cycloalkylthio, C₁-C₈ alkylamino, C₁-C₈ haloalkylamino, C₂-C₈ dialkylamino, C₂-C₈ halodialkylamino, C₃-C₈ cycloalkylamino, C₁-C₈ alkylsulfonylamino, C₁-C₈ halo alkylsulfonylamino, C₂-C₈ alkylcarbonylamino, C₂-C₈ haloalkylcarbonylamino, C₃-C₁₀ trialkylsilyl, pyrrolidinyl, piperidinyl or morpholinyl;
R² is H, amino, cyano, halogen, —CH(═O), —C(═O)OH, —C(═O)NH₂, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₃-C₆ cycloalkenyl, C₃-C₆ halocycloalkenyl, C₄-C₆ alkylcycloalkyl, C₄-C₆ cycloalkylalkyl, C₄-C₆ halocycloalkylalkyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₄-C₆ cycloalkoxycarbonyl, C₅-C₆ cycloalkylalkoxycarbonyl, C₂-C₆ alkylaminocarbonyl, C₃-C₆ dialkylaminocarbonyl, C₁-C₆ alkoxy, C₁-C₆ haloalkoxy, C₂-C₆ alkenyloxy, C₂-C₆ haloalkenyloxy, C₂-C₆ alkynyloxy, C₃-C₆ haloalkynyloxy, C₃-C₆ cycloalkoxy, C₃-C₆ halocycloalkoxy, C₂-C₆ alkoxyalkoxy, C₂-C₆ alkylcarbonyloxy, C₂-C₆ haloalkylcarbonyloxy, C₁-C₆ alkylthio, C₁-C₆ haloalkylthio, C₃-C₆ cycloalkylthio, C₁-C₆ alkylamino, C₂-C₆ dialkylamino, C₁-C₆ haloalkylamino, C₂-C₆ halodialkylamino, C₃-C₆ cycloalkylamino, C₁-C₆ alkylsulfonylamino, C₁-C₆ halo alkylsulfonylamino C₂-C₆ alkylcarbonylamino or C₂-C₆ haloalkylcarbonylamino;
R³ is H, cyano, halogen, hydroxy, C₁-C₃ alkyl, C₁-C₃ haloalkyl, C₁-C₃ alkoxy or C₁-C₃ haloalkoxy; or
R² and R³ are taken together with the carbon atom to which they are attached to form a 3- to 7-membered ring containing ring members selected from carbon atoms and up to 4 heteroatoms independently selected from up to 2 O, up to 2 S and up to 2 N atoms, wherein up to 3 carbon atom ring members are independently selected from C(═O) and C(═S), and the sulfur atom ring members are independently selected from S(═O)ₛ(═NR²⁰)_{f}, the ring optionally substituted with up to 4 substituents independently selected from halogen, cyano, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
R⁴ is optionally substituted phenyl, optionally substituted naphthalenyl or an optionally substituted 5- to 6-membered heteroaromatic ring; or H, cyano, halogen, hydroxy, —CH(═O), C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₁-C₄ alkoxy, C₁-C₄ haloalkoxy, C₂-C₄ alkylcarbonyloxy, C₂-C₄ haloalkylcarbonyloxy, C₂-C₅ alkoxycarbonyloxy, C₂-C₅ alkylaminocarbonyloxy, C₃-C₅ dialkylaminocarbonyloxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl C₂-C₄ alkylcarbonyl, C₂-C₄ haloalkylcarbonyl, C₂-C₅ alkoxycarbonyl, C₂-C₅ alkylaminocarbonyl or C₃-C₅ dialkylaminocarbonyl;
R⁵ is H, C₁-C₃ alkyl or C₁-C₃ haloalkyl;
each R^{7a} is independently halogen, cyano, hydroxy, C₁-C₄ alkyl, C₁-C₄ haloalkyl, C₂-C₄ alkenyl or C₁-C₄ alkoxy; or
two R^{7a} are taken together as C₁-C₄ alkylene or C₂-C₄ alkenylene to form a bridged or fused ring system;
R^{7b} is H, cyano, C₁-C₃ alkyl, C₁-C₃ haloalkyl, C₃-C₆ cycloalkyl, C₁-C₃ alkoxy, C₂-C₃ alkylcarbonyl or C₂-C₃ alkoxycarbonyl;
each R⁸ is independently cyano, halogen, hydroxy, methyl or methoxy;
each R^{9a} is independently cyano, halogen, hydroxy, C₁-C₄ alkyl, C₁-C₄ haloalkyl, C₃-C₆ cycloalkyl, C₂-C₄ alkoxyalkyl, C₁-C₄ alkoxy, C₁-C₄ haloalkoxy, C₂-C₄ alkylcarbonyl or C₂-C₄ alkoxycarbonyl;
each R^{9b} is independently cyano, C₁-C₄ alkyl, C₁-C₄ haloalkyl, C₃-C₆ cycloalkyl, C₁-C₄ alkoxy, C₂-C₄ alkylcarbonyl or C₂-C₄ alkoxycarbonyl;
each R^{10a} is independently amino, cyano, halogen, hydroxy, nitro, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₄ hydroxyalkyl, C₃-C₆ cycloalkyl, C₃-C₆ halocycloalkyl, C₄-C₁₀ cycloalkylalkyl, C₄-C₁₀ alkylcycloalkyl, C₅-C₁₀ alkylcycloalkylalkyl, C₆-C₁₄ cycloalkylcycloalkyl, C₂-C₄ alkoxyalkyl, C₁-C₄ alkoxy, C₁-C₄ haloalkoxy, C₂-C₆ alkylcarbonyloxy, C₁-C₄ alkylthio, C₁-C₄ haloalkylthio, C₂-C₆ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or
phenyl or naphthalenyl, each optionally substituted with up to 3 substituents independently selected from cyano, halogen, C₁-C₂ alkyl, C₁-C₂ haloalkyl, C₁-C₂ alkoxy and C₁-C₂ haloalkoxy; or
a 5- to 6-membered heteroaromatic ring containing ring members selected from carbon atoms and up to 4 heteroatoms independently selected from up to 2 O, up to 2 S and up to 4 N atoms, the ring optionally substituted with up to 3 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members; or
a 3- to 7-membered nonaromatic ring containing ring members selected from carbon atoms and up to 4 heteroatoms independently selected from up to 2 O, up to 2 S and up to 4 N atoms, wherein up to 3 carbon atom ring members are independently selected from C(═O) and C(═S), the ring optionally substituted with up to 3 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
R^{10b} is cyano, C₁-C₃ alkyl, C₁-C₃ haloalkyl, C₃-C₆ cycloalkyl C₁-C₃ alkoxy, C₂-C₃ alkylcarbonyl or C₂-C₃ alkoxycarbonyl;
R¹¹ is H, cyano, halogen, hydroxy, —CH(═O), C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₃-C₅ alkoxycarbonylalkyl, C₂-C₄ alkylcarbonyl, C₂-C₄ haloalkylcarbonyl, C₂-C₅ alkoxycarbonyl, C₂-C₅ alkylaminocarbonyl, C₃-C₅ dialkylaminocarbonyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
R¹² is H, C₁-C₄ alkyl, C₁-C₄ haloalkyl, C₂-C₄ alkenyl, C₂-C₄ haloalkenyl, C₃-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₃-C₅ alkoxycarbonylalkyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₂-C₅ alkylaminocarbonyl or C₃-C₅ dialkylaminocarbonyl;
R¹³ is H, cyano, C₁-C₄ alkyl, C₁-C₄ haloalkyl, C₂-C₄ alkoxyalkyl, C₂-C₄ alkylthioalkyl, C₁-C₄ alkylsulfonyl, C₁-C₄ haloalkylsulfonyl, C₂-C₄ alkylcarbonyl, C₂-C₄ haloalkylcarbonyl, C₂-C₄ alkoxycarbonyl, C₂-C₄ alkylaminocarbonyl or C₃-C₅ dialkylaminocarbonyl; or
R¹³ and R³ are taken together with the atoms to which they are attached to form a 5- to 7-membered partially saturated ring containing ring members selected from carbon atoms and up to 3 heteroatoms independently selected from up to 1 O, up to 1 S and up to 1 N atom, the ring optionally substituted with up to 3 substituents independently selected from cyano, halogen, nitro, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
each R¹⁴ is independently H, C₁-C₃ alkyl or C₁-C₃ haloalkyl;
R¹⁵ and R¹⁶ are each C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₃-C₆ halocycloalkyl, C₄-C₈ alkylcycloalkyl, C₄-C₈ cycloalkylalkyl, C₄-C₈ halocycloalkylalkyl, C₅-C₈ alkylcycloalkylalkyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₂-C₆ alkylaminocarbonyl, C₃-C₈ dialkylaminocarbonyl or C₄-C₈ cycloalkylaminocarbonyl;
R¹⁷ is H, amino, cyano, hydroxy, C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₄-C₈ cycloalkylalkyl, C₂-C₆ alkoxyalkyl, C₁-C₆ alkoxy, C₁-C₆ haloalkoxy, C₁-C₆ alkylsulfonyl, C₁-C₆ haloalkylsulfonyl, C₁-C₆ alkylamino, C₁-C₆ haloalkylamino, C₂-C₈ dialkylamino, C₂-C₈ halodialkylamino, C₂-C₆ alkylcarbonyl or C₂-C₆ haloalkylcarbonyl;
R¹⁸ is H, C₁-C₆ alkyl, C₁-C₆ haloalkyl, C₃-C₆ alkenyl, C₃-C₆ alkynyl or C₃-C₆ cycloalkyl; or
R¹⁷ and R¹⁸ are taken together as —(CH₂)₄—, —(CH₂)₅— or —(CH₂)₂O(CH₂)₂—;
R¹⁹ is H, cyano, halogen, C₁-C₄ alkyl, C₁-C₄ haloalkyl, C₂-C₄ alkoxyalkyl, C₂-C₄ alkylcarbonyl, C₂-C₄ alkoxycarbonyl, C₂-C₃ alkylaminocarbonyl or C₃-C₆ dialkylaminocarbonyl;
each R²⁰ is independently H, cyano, C₁-C₆ alkyl, C₁-C₆ haloalkyl, C₃-C₈ cycloalkyl, C₃-C₈ halocycloalkyl, C₁-C₆ alkoxy, C₁-C₆ haloalkoxy, C₁-C₆ alkylamino, C₂-C₈ dialkylamino, C₁-C₆ haloalkylamino or phenyl;
n is 0, 1 or 2; and
s and f are independently 0, 1 or 2 in each instance of S(═O)ₛ(═NR²⁰)_{f}; provided that:
(a) that the sum of s and f is 0, 1 or 2; and
(b) when A is C(═O) or CH(R¹¹) and R¹¹ is hydroxy, then R¹ is bonded through a carbon atom to A.

More particularly, this invention pertains to a compound of Formula 1 (including all geometric and stereoisomers), tautomers, an N-oxide, or a salt thereof.

This invention also relates to a fungicidal composition comprising (a) a compound of the invention (i.e. in a fungicidally effective amount); and (b) at least one additional component selected from the group consisting of surfactants, solid diluents and liquid diluents.

This invention also relates to a fungicidal composition comprising (a) a compound of Formula 1; and (b) at least one other fungicide (e.g., at least one other fungicide having a different site of action).

This invention further relates to a method for controlling plant diseases caused by fungal plant pathogens comprising applying to the plant or portion thereof, or to the plant seed, a fungicidally effective amount of a compound of the invention (e.g., as a composition described herein).

### DETAILS OF THE INVENTION

As used herein, the terms “comprises,” “comprising,” “includes,” “including,” “has,” “having,” “contains,” “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As referred to in the present disclosure and claims, “plant” includes members of Kingdom Plantae, particularly seed plants (Spermatopsida), at all life stages, including young plants (e.g., germinating seeds developing into seedlings) and mature, reproductive stages (e.g., plants producing flowers and seeds). Portions of plants include geotropic members typically growing beneath the surface of the growing medium (e.g., soil), such as roots, tubers, bulbs and corms, and also members growing above the growing medium, such as foliage (including stems and leaves), flowers, fruits and seeds.

As referred to herein, the term “seedling”, used either alone or in a combination of words means a young plant developing from the embryo of a seed or bud of a vegetative propagation unit such as tuber, corm or rhizome.

As referred to herein, the term “broadleaf” used either alone or in words such as “broadleaf crop” means dicot or dicotyledon, a term used to describe a group of angiosperms characterized by embryos having two cotyledons.

Generally when a molecular fragment (i.e. radical) is denoted by a series of atom symbols (e.g., C, H, N, O, S) the implicit point or points of attachment will be easily recognized by those skilled in the art. In some instances herein, particularly when alternative points of attachment are possible, the point or points of attachment may be explicitly indicated by a hyphen (“-”).

In the above recitations, the term “alkyl”, used either alone or in compound words such as “alkylthio” or “haloalkyl” includes straight-chain and branched alkyl, such as, methyl, ethyl, n-propyl, i-propyl, and the different butyl, pentyl and hexyl isomers. “Alkenyl” includes straight-chain and branched alkenes such as ethenyl, 1-propenyl, 2-propenyl, and the different butenyl, pentenyl and hexenyl isomers. “Alkenyl” also includes polyenes such as 1,2-propadienyl and 2,4-hexadienyl. “Alkynyl” includes straight-chain and branched alkynes such as ethynyl, 1-propynyl, 2-propynyl, and the different butynyl, pentynyl and hexynyl isomers. “Alkynyl” can also include moieties comprised of multiple triple bonds such as 2,5-hexadiynyl. “Alkylene” denotes a straight-chain or branched alkanediyl. Examples of “alkylene” include CH₂, CH₂CH₂, CH(CH₃), CH₂CH₂CH₂, CH₂CH(CH₃), and the different butylene isomers. “Alkenylene” denotes a straight-chain or branched alkenediyl containing one olefinic bond. Examples of “alkenylene” include CH═CH, CH₂CH═CH and CH═C(CH₃).

“Alkoxy” includes, for example, methoxy, ethoxy, n-propyloxy, i-propyloxy, and the different butoxy, pentoxy and hexyloxy isomers. “Alkenyloxy” includes straight-chain and branched alkenyl attached to and linked through an oxygen atom. Examples of “alkenyloxy” include H₂C═CHCH₂O, CH₃CH═CHCH₂O and (CH₃)₂C≡CHCH₂O. “Alkynyloxy” includes straight-chain and branched alkynyloxy moieties. Examples of “alkynyloxy” include HC≡CCH₂O, CH₃C≡CCH₂O and CH₃C≡CCH₂CH₂O. The term “alkylthio” includes straight-chain and branched alkylthio moieties such as methylthio, ethylthio, and the different propylthio, butylthio, pentylthio and hexylthio isomers. “Alkylsulfinyl” includes both enantiomers of an alkylsulfinyl group. Examples of “alkylsulfinyl” include CH₃S(═O), CH₃CH₂S(═O), CH₃CH₂CH₂S(═O), (CH₃)₂CHS(═O), and the different butylsulfinyl, pentylsulfinyl and hexylsulfinyl isomers. Examples of “alkylsulfonyl” include CH₃S(═O)₂, CH₃CH₂S(—O)₂, CH₃CH₂CH₂S(═O)₂, (CH₃)₂CHS(═O)₂, and the different butylsulfonyl, pentylsulfonyl and hexylsulfonyl isomers. “Alkylamino” includes an NH radical substituted with a straight-chain or branched alkyl group. Examples of “alkylamino” include CH₃CH₂NH, CH₃CH₂CH₂NH, and (CH₃)₂CHCH₂NH. Examples of “dialkylamino” include (CH₃)₂N, (CH₃CH₂CH₂)₂N and CH₃CH₂(CH₃)N.

“Alkylcarbonyl” denotes a straight-chain or branched alkyl group bonded to a C(═O) moiety. Examples of “alkylcarbonyl” include CH₃C(═O), CH₃CH₂CH₂C(═O) and (CH₃)₂CHC(═O). Examples of “alkoxycarbonyl” include CH₃OC(═O), CH₃CH₂OC(═O), CH₃CH₂CH₂OC(═O), (CH₃)₂CHOC(═O), and the different butoxy- and pentoxycarbonyl isomers. Examples of “alkylaminocarbonyl” include CH₃NHC(═O), CH₃CH₂NHC(═O), CH₃CH₂CH₂NHC(═O), (CH₃)₂CHNHC(═O), and the different butylamino- and pentylaminocarbonyl isomers. Examples of “dialkylaminocarbonyl” include (CH₃)₂NC(═O), (CH₃CH₂)₂NC(═O), CH₃CH₂(CH₃)NC(═O), (CH₃)₂CH(CH₃)NC(═O) and CH₃CH₂CH₂(CH₃)NC(═O).

“Alkoxyalkyl” denotes alkoxy substitution on alkyl. Examples of “alkoxyalkyl” include CH₃OCH₂, CH₃OCH₂CH₂, CH₃CH₂OCH₂, CH₃CH₂CH₂CH₂OCH₂ and CH₃CH₂OCH₂CH₂. “Alkoxyalkoxy” denotes alkoxy substitution on another alkoxy moiety. “Alkoxyalkoxyalkyl” denotes alkoxyalkoxy substitution on alkyl. Examples of “alkoxyalkoxyalkyl” include CH₃OCH₂OCH₂ CH₃OCH₂OCH₂CH₂ and CH₃CH₂OCH₂OCH₂.

“Alkylthioalkyl” denotes alkylthio substitution on alkyl. Examples of “alkylthioalkyl” include CH₃SCH₂, CH₃SCH₂CH₂, CH₃CH₂SCH₂, CH₃CH₂CH₂CH₂SCH₂ and CH₃CH₂SCH₂CH₂; “alkylsulfinylalkyl” and “alkylsulfonylalkyl” include the corresponding sulfoxides and sulfones, respectively. “Alkylcarbonylthio” denotes a straight-chain or branched alkylcarbonyl attached to and linked through a sulfur atom. Examples of “alkylcarbonylthio” include CH₃C(═O)S, CH₃CH₂CH₂C(═O)S and (CH₃)₂CHC(═O)S.

“Alkylaminoalkyl” denotes alkylamino substitution on alkyl. Examples of “alkylaminoalkyl” include CH₃NHCH₂, CH₃NHCH₂CH₂, CH₃CH₂NHCH₂, CH₃CH₂CH₂CH₂NHCH₂ and CH₃CH₂NHCH₂CH₂. Examples of “dialkylaminoalkyl” include ((CH₃)₂CH))₂NCH₂, (CH₃CH₂CH₂)₂NCH₂ and CH₃CH₂(CH₃)NCH₂CH₂.

The term “alkylcarbonylamino” denotes alkyl bonded to a C(═O)NH moiety. Examples of “alkylcarbonylamino” include CH₃CH₂C(═O)NH and CH₃CH₂CH₂C(═O)NH. “Alkylsulfonylamino” denotes an NH radical substituted with alkylsulfonyl. Examples of “alkylsulfonylamino” include CH₃CH₂S(═O)₂NH and (CH₃)₂CHS(═O)₂NH.

The term “alkylcarbonyloxy” denotes a straight-chain or branched alkyl bonded to a C(═O)O moiety. Examples of “alkylcarbonyloxy” include CH₃CH₂C(═O)O and (CH₃)₂CHC(═O)O. Examples of “alkoxycarbonyloxy” include CH₃CH₂CH₂OC(═O)O and (CH₃)₂CHOC(═O)O. The term “alkoxycarbonylalkyl” denotes alkoxycarbonyl substitution on alkyl. Examples of “alkoxycarbonylalkyl” include CH₃CH₂OC(═O)CH₂, (CH₃)₂CHCH₂OC(═O)CH₂ and CH₃OC(═O)CH₂CH₂.

The term “alkylaminocarbonyloxy” denotes a straight-chain or branched alkylaminocarbonyl attached to and linked through an oxygen atom. Examples of “alkylaminocarbonyloxy” include (CH₃)₂CHCH₂NHC(═O)O and CH₃CH₂NHC(═O)O. Examples of “dialkylaminocarbonyloxy” include CH₃CH₂CH₂(CH₃)NC(═O)O and (CH₃)₂NC(═O)O.

“Cycloalkyl” includes, for example, cyclopropyl, cyclobutyl, cyclopentyl and cyclohexyl. The term “cycloalkylalkyl” denotes cycloalkyl substitution on an alkyl moiety. Examples of “cycloalkylalkyl” include cyclopropylmethyl, cyclopentylethyl, and other cycloalkyl moieties bonded to a straight-chain or branched alkyl group. The term “alkylcycloalkyl” denotes alkyl substitution on a cycloalkyl moiety and includes, for example, ethylcyclopropyl, i-propylcyclobutyl, methylcyclopentyl and methylcyclohexyl. “Cycloalkenyl” includes groups such as cyclopentenyl and cyclohexenyl as well as groups with more than one double bond such as 1,3- or 1,4-cyclohexadienyl.

The term “cycloalkoxy” denotes cycloalkyl attached to and linked through an oxygen atom such as cyclopentyloxy and cyclohexyloxy. The term “cycloalkylthio” denotes cycloalkyl attached to and linked through a sulfur atom such as cyclopropylthio and cyclopentylthio. The term “cycloalkoxyalkyl” denotes cycloalkoxy substitution on an alkyl moiety. Examples of “cycloalkoxyalkyl” include cyclopropyloxymethyl, cyclopentyloxyethyl, and other cycloalkoxy groups bonded to a straight-chain or branched alkyl moiety. “Cycloalkylalkoxy” denotes cycloalkyl substitution on an alkoxy moiety. Examples of “cycloalkylalkoxy” include cyclopropylmethoxy, cyclopentylethoxy, and other cycloalkyl groups bonded to a straight-chain or branched alkoxy moiety.

“Alkylcycloalkylalkyl” denotes an alkyl group substituted with alkylcycloalkyl. Examples of “alkylcycloalkylalkyl” include methylcyclohexylmethyl and ethylcycloproylmethyl. The term “cycloalkylcycloalkyl” denotes cycloalkyl substitution on another cycloalkyl ring, wherein each cycloalkyl ring independently has from 3 to 7 carbon atom ring members. Examples of cycloalkylcycloalkyl include cyclopropylcyclopropyl (such as 1,1′-bicyclopropyl-1-yl, 1,1′-bicyclopropyl-2-yl), cyclohexylcyclopentyl (such as 4-cyclopentylcyclohexyl) and cyclohexylcyclohexyl (such as 1,1′-bicyclohexyl-1-yl), and the different cis- and trans-cycloalkylcycloalkyl isomers, (such as (1R,2S)-1,1′-bicyclopropyl-2-yl and (1R,2R)-1,1′-bicyclopropyl-2-yl).

“Cycloalkylamino” denotes an NH radical substituted with cycloalkyl. Examples of “cycloalkylamino” include cyclopropylamino and cyclohexylamino. The term “cycloalkylaminoalkyl” denotes cycloalkylamino substitution on an alkyl group. Examples of “cycloalkylaminoalkyl” include cyclopropylaminomethyl, cyclopentylaminoethyl, and other cycloalkylamino moieties bonded to a straight-chain or branched alkyl group.

“Cycloalkylcarbonyl” denotes cycloalkyl bonded to a C(═O) group including, for example, cyclopropylcarbonyl and cyclopentylcarbonyl. The term “cycloalkoxycarbonyl” means cycloalkoxy bonded to a C(═O) group, for example, cyclopropyloxycarbonyl and cyclopentyloxycarbonyl. “Cycloalkylaminocarbonyl” denotes cycloalkylamino bonded to a C(═O) group, for example, cyclopentylaminocarbonyl and cyclohexylaminocarbonyl. “Cycloalkylalkoxycarbonyl” denotes cycloalkylalkoxy bonded to a C(═O) group. Examples of “cycloalkylalkoxycarbonyl” include cyclopropylethoxycarbonyl and cyclobutylmethoxycarbonyl.

The term “halogen”, either alone or in compound words such as “haloalkyl”, or when used in descriptions such as “alkyl substituted with halogen” includes fluorine, chlorine, bromine or iodine. Further, when used in compound words such as “haloalkyl”, or when used in descriptions such as “alkyl substituted with halogen” said alkyl may be partially or fully substituted with halogen atoms which may be the same or different. Examples of “haloalkyl” or “alkyl substituted with halogen” include F₃C, F₂HC, ClCH₂, CF₃CH₂ and CF₃CCl₂. The terms “haloalkenyl”, “haloalkynyl” “haloalkoxy”, “haloalkylthio”, “haloalkylamino”, “haloalkylsulfinyl”, “haloalkylsulfonyl”, “halocycloalkyl”, and the like, are defined analogously to the term “haloalkyl”. Examples of “haloalkenyl” include Cl₂C≡CHCH₂ and CF₃CH₂CH═CHCH₂. Examples of “haloalkynyl” include HC≡CCHCl, CF₃C≡C, CCl₃C≡C and FCH₂C≡CCH₂. Examples of “haloalkoxy” include CF₃O, CCl₃CH₂O, F₂CHCH₂CH₂O and CF₃CH₂O. Examples of “haloalkylthio” include CCl₃S, CF₃S, CCl₃CH₂S and ClCH₂CH₂CH₂S. Examples of “haloalkylamino” include CF₃(CH₃)CHNH, (CF₃)₂CHNH and CH₂ClCH₂NH. Examples of “haloalkylsulfinyl” include CF₃S(═O), CCl₃S(═O), CF₃CH₂S(═O) and CF₃CF₂S(═O). Examples of “haloalkylsulfonyl” include CF₃S(—O)₂, CCl₃S(—O)₂, CF₃CH₂S(—O)₂ and CF₃CF₂S(—O)₂. Examples of “halocycloalkyl” include 2-chlorocyclopropyl, 2-fluorocyclobutyl, 3-bromocyclopentyl and 4-chorocyclohexyl. The term “halodialkyl”, either alone or in compound words such as “halodialkylamino”, means at least one of the two alkyl groups is substituted with at least one halogen atom, and independently each halogenated alkyl group may be partially or fully substituted with halogen atoms which may be the same or different. Examples of “halodialkylamino” include (BrCH₂CH₂)₂N and BrCH₂CH₂(ClCH₂CH₂)N.

“Hydroxyalkyl” denotes an alkyl group substituted with one hydroxy group. Examples of “hydroxyalkyl” include HOCH₂CH₂, CH₃CH₂(OH)CH and HOCH₂CH₂CH₂CH₂.

“Trialkylsilyl” includes 3 branched and/or straight-chain alkyl radicals attached to and linked through a silicon atom, such as trimethylsilyl, triethylsilyl and tert-butyldimethylsilyl.

The total number of carbon atoms in a substituent group is indicated by the “Cᵢ-Cⱼ” prefix where i and j are numbers from 1 to 14. For example, C₁-C₄ alkylsulfonyl designates methylsulfonyl through butylsulfonyl; C₂ alkoxyalkyl designates CH₃OCH₂; C₃ alkoxyalkyl designates, for example, CH₃CH(OCH₃), CH₃OCH₂CH₂ or CH₃CH₂OCH₂; and C₄ alkoxyalkyl designates the various isomers of an alkyl group substituted with an alkoxy group containing a total of four carbon atoms, examples include CH₃CH₂CH₂OCH₂ and CH₃CH₂OCH₂CH₂.

The term “unsubstituted” in connection with a group such as a ring or ring system means the group does not have any substituents other than its one or more attachments to the remainder of Formula 1. The term “optionally substituted” means that the number of substituents can be zero. Unless otherwise indicated, optionally substituted groups may be substituted with as many optional substituents as can be accommodated by replacing a hydrogen atom with a non-hydrogen substituent on any available carbon or nitrogen atom. Commonly, the number of optional substituents (when present) ranges from 1 to 3. As used herein, the term “optionally substituted” is used interchangeably with the phrase “substituted or unsubstituted” or with the term “(un)substituted.” The term “optionally substituted” without recitation of number or identity of possible substituents (e.g., phenyl and naphthalenyl in definition of R¹ and R⁶) refers to groups which are unsubstituted or have at least one non-hydrogen substituent that does not extinguish the biological activity possessed by the unsubstituted analog.

The number of optional substituents may be restricted by an expressed limitation. For example, the phrase “optionally substituted with up to 3 substituents independently selected from R^{10a}” means that 0, 1, 2 or 3 substituents can be present (if the number of potential connection points allows). When a range specified for the number of substituents (e.g., p being an integer from 1 to 3 in Exhibit 2) exceeds the number of positions available for the substituents on a group (e.g., 2 positions available for (R₁₀ₐ)ₚ on Q-4 in Exhibit 2), then the actual higher end of the range is recognized to be the number of available positions.

When a compound is substituted with a substituent bearing a subscript that indicates the number of said substituents can vary (e.g., (R^{10a})ₚ in Exhibit 2 wherein p is 1 to 3), then said substituents are independently selected from the group of defined substituents, unless otherwise indicated. When a variable group is shown to be optionally attached to a position, for example (R^{10a})ₚ in Exhibit 2 wherein p may be 0, then hydrogen may be at the position even if not recited in the definition of the variable group.

Naming of substituents in the present disclosure uses recognized terminology providing conciseness in precisely conveying to those skilled in the art the chemical structure. For sake of conciseness, locant descriptors may be omitted.

Unless otherwise indicated, a “ring” or “ring system” as a component of Formula 1 is carbocyclic or heterocyclic. The term “ring system” denotes two or more connected rings. The term “bicyclic ring system” denotes a ring system consisting of two rings sharing two or more common atoms. In a “fused bicyclic ring system” the common atoms are adjacent, and therefore the rings share two adjacent atoms and a bond connecting them. In a “bridged bicyclic ring system” the common atoms are not adjacent (i.e. there is no bond between the bridgehead atoms). A “bridged bicyclic ring system” can be formed by bonding a segment of one or more atoms to nonadjacent ring members of a ring.

The term “ring member” refers to an atom (e.g., C, O, N or S) or other moiety (e.g., C(═O), C(═S) or S(═O)ₛ(═NR²⁰)_{f}) forming the backbone of a ring or ring system. The term “aromatic” indicates that each ring atom is essentially in the same plane and has a p-orbital perpendicular to the ring plane, and that (4n+2) π electrons, where n is a positive integer, are associated with the ring to comply with Hückel's rule.

The term “carbocyclic ring” denotes a ring wherein the atoms forming the ring backbone are selected only from carbon. Unless otherwise indicated, a carbocyclic ring can be a saturated, partially unsaturated, or fully unsaturated ring. When a fully unsaturated carbocyclic ring satisfies Hückel's rule, then said ring is also called an “aromatic ring”. “Saturated carbocyclic” refers to a ring having a backbone consisting of carbon atoms linked to one another by single bonds; unless otherwise specified, the remaining carbon valences are occupied by hydrogen atoms.

As used herein, the terms “partially unsaturated ring” or “partially unsaturated heterocycle” refer to a ring which contain unsaturated ring atoms and one or more double bonds but is not aromatic. The term “nonaromatic” includes rings that are fully saturated as well as partially or fully unsaturated, provided that the rings are not aromatic.

The terms “heterocyclic ring”, “heterocycle” or “heteroaromatic bicyclic ring system” denote a ring wherein at least one of the atoms forming the ring backbone is other than carbon. Unless otherwise indicated, a heterocyclic ring can be a saturated, partially unsaturated, or fully unsaturated ring. When a fully unsaturated heterocyclic ring satisfies Hückel's rule, then said ring is also called a “heteroaromatic ring” or aromatic heterocyclic ring. “Saturated heterocyclic ring” refers to a heterocyclic ring containing only single bonds between ring members. The terms “heteroaromatic ring system” or “heteroaromatic bicyclic ring system” denote a ring wherein at least one of the atoms forming the ring backbone is other than carbon and at least one ring is aromatic. Unless otherwise indicated, heterocyclic rings and heteroaromatic ring systems can be attached through any available carbon or nitrogen by replacement of a hydrogen on said carbon or nitrogen.

The wavy bond between the nitrogen atom and the atom represented by A¹ means a single bond and the geometry about the adjacent double (i.e. the bond linking the nitrogen atom to the substituents R² and R³) is either cis-(Z), trans-(E), or a mixture thereof.

As described above, G forms a 5- to 6-membered ring including as ring members the two carbon atoms indentified as “q” and “r” in Formula 1. The other 3 to 4 ring members (i.e. the intervening linking atoms) are selected from carbon atoms and up to 2 heteroatoms independently selected from up to 1 O, up to 1 S and up to 2 N atoms, wherein up to 1 carbon atom ring member is selected from C(═O), C(═S) and C(═NOH), the ring optionally substituted with up to 2 substituents independently selected from R⁸ on carbon atom ring members and methyl on nitrogen atom ring members. In this definition the ring members selected from up to 1 O, up to 1 S and up to 2 N atoms are optional, because the number of heteroatom ring members may be zero. The nitrogen atom ring members may be oxidized as N-oxides, because compounds relating to Formula 1 also include N-oxide derivatives. The up to 1 carbon atom ring member selected from C(═O), C(═S) and C(═NOH) are in addition to the up to 2 heteroatoms selected from up to 1 O, up to 1 S and up to 2 N atoms. The optional substituents (when present) are attached to available carbon and nitrogen atom ring members of the intervening linking atoms.

As described above, Z is a saturated, partially unsaturated or fully unsaturated chain containing 1- to 3-atoms selected from up to 3 carbon, up to 1 O, up to 1 S and up to 2 N atoms. When Z is denoted as a chain consisting of a series of atoms wherein alternative points of attachment are possible (e.g., Z is OCH₂CH₂ or NOCH₂), then the atom on the left is connected to G-ring and the atom on the right is connected to Q in Formula 1 (i.e. G-OCH₂CH₂-Q and G=NOCH₂-Q). When Z is denoted as a radical wherein alternative bonds of attachment are possible (e.g., Z is CH), then both configurations are allowed (i.e. G=CH—Z or G-CH═Z), unless otherwise indicated. Note in some instances the G-ring is denoted as a radical wherein its connection to Z is indicated as a single bond or a double bond (e.g., G-1 and G-2 in Exhibit 1), in those instances one skilled in the art can easily determine how to select an appropriate Z group.

As described above, Q is (inter alia) a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, each ring or ring system optionally substituted with up to 3 substituents independently selected from R^{10a} on carbon and R^{10b} nitrogen atom ring members. In this definition the nitrogen atom ring members may be oxidized as N-oxides, because compounds relating to Formula 1 also include N-oxide derivatives. As R^{10a} and R^{10b} are optional, 0 to 3 substituents may be present, limited only by the number of available points of attachment.

As described above, Q is (inter alia) a 3- to 7-membered nonaromatic carbocyclic ring, a 5- to 7-membered nonaromatic heterocyclic ring or an 8- to 11-membered nonaromatic bicyclic ring system, each ring or ring system containing ring members selected from carbon atoms and up to 4 heteroatoms independently selected from up to 2 O, up to 2 S and up to 4 N atoms, wherein up to 3 carbon atom ring members are independently selected from C(═O) and C(═S) and the sulfur atom ring members are independently selected from S(═O)ₛ(═NR²⁰)_{f}, each ring or ring system optionally substituted with up to 3 substituents independently selected from R^{10a} on carbon and R^{10b} nitrogen atom ring members. In this definition when no heteroatom ring members are present, the ring or ring system is carbocyclic. If at least one heteroatom ring member is present, the ring or ring system is heterocyclic. The definition of S(═O)ₛ(═NR²⁰)_{f} allows up to 2 sulfur ring members, which can be oxidized sulfur moieties (e.g., S(═O) or S(═O)₂) or aminated moieties (e.g., S(═NR²⁰)) or unoxidized sulfur atoms (i.e. when s and f are both zero). The nitrogen atom ring members may be oxidized as N-oxides, because compounds relating to Formula 1 also include N-oxide derivatives. The up to 3 carbon atom ring members selected from C(═O) and C(═S) are in addition to the up to 4 heteroatoms selected from up to 2 O, up to 2 S and up to 4 N atoms.

As described above, R² and R³ may be taken together with the carbon atom to which they are directly attached to form a 3- to 7-membered ring. Thus, the 3- to 7-membered ring includes as a ring member the carbon atom to which the substituents R² and R³ are attached. The other 2 to 6 ring members are selected from carbon atoms and up to 4 heteroatoms independently selected from up to 2 O, up to 2 S, and up to 2 N atoms, wherein up to 3 carbon atom ring members are independently selected from C(═O) and C(═S), and the sulfur atom ring members are independently selected from S(═O)ₛ(═NR²⁰)_{f}, the ring optionally substituted with up to with up to 4 substituents independently selected from halogen, cyano, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members. In this definition the heteroatoms are optional, because the number of heteroatom ring members may be zero. When no heteroatom ring member is present, the ring is carbocyclic. If at least one heteroatom ring member is present, the ring is heterocyclic. The definition of S(═O)ₛ(═NR²⁰)_{f} allows up to 2 sulfur ring members, which can be oxidized sulfur moieties (e.g., S(═O) or S(═O)₂) or aminated moieties (e.g., S(═NR²⁰)) or unoxidized sulfur atoms (i.e. when s and f are both zero). The nitrogen atom ring members may be oxidized as N-oxides, because compounds relating to Formula 1 also include N-oxide derivatives. The ring is optionally substituted with up to 4 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members.

As described above, R³ and R¹³ may be taken together with the linking atoms to which they are directly attached to form a 5- to 7-membered partially unsaturated ring. Thus, the 5- to 7-membered ring includes as a ring member the carbon atom to which R³ is directly attached, the nitrogen atom in Formula 1 depicted as “═N˜” and the nitrogen atom to which R¹³ is directly attached. The other 2 to 4 ring members of the ring are selected from up to 1 O, up to 1 S and up to 1 N atom. In this definition the ring members selected from up to 1 O, up to 1 S and up to 1 N atom are optional, because the number of heteroatom ring members may be zero. The ring is optionally substituted with up to 3 substituents independently selected from cyano, halogen, nitro, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members. These optional substituents (when present) are attached to available carbon and nitrogen atom ring members in the portion of the ring provided by R³ and R¹³. The nitrogen atom ring members may be oxidized as N-oxides, because compounds relating to Formula 1 also include N-oxide derivatives.

As described above, A is CH(R¹¹), N(R¹²) or C(═O), provided that when A is C(═O) or CH(R¹¹) and R¹¹ is hydroxy, then R¹ is bonded through a carbon atom to A. Thus, this definition does not include the possibility of “—R¹—C(═O)—” or “—R¹—CH(OH)—” wherein R¹ is connected via a nitrogen atom.

Compounds of Formula 1 can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers. Compounds of Formula 1 may be present as a mixture of stereoisomers, individual stereoisomers, or as an optically active form.

Compounds of Formula 1 can exist as one or more conformational isomers due to restricted rotation about an amide bond (e.g., C(═W)—N) in Formula 1. Compounds of Formula 1 comprise mixtures of conformational isomers. In addition, compounds of Formula 1 include compounds that are enriched in one conformer relative to others.

Molecular depictions drawn herein follow standard conventions for depicting stereochemistry. To indicate stereoconfiguration, bonds rising from the plane of the drawing and towards the viewer are denoted by solid wedges where the broad end of the wedge is attached to the atom rising from the plane of the drawing towards the viewer. Bonds going below the plane of the drawing and away from the viewer are denoted by dashed wedges where the narrow end of the wedge is attached to the atom further away from the viewer. Constant width lines indicate bonds with a direction opposite or neutral relative to bonds shown with solid or dashed wedges; constant width lines also depict bonds in molecules or parts of molecules in which no particular stereoconfiguration is intended to be specified.

One skilled in the art recognizes that compounds of Formula 1 can exist in equilibrium with one or more of its respective tautomeric counterparts. Unless otherwise indicated, reference to a compound by one tautomer description is to be considered to include all tautomers. For example, when E is E² and R³ is hydroxy, then reference to the tautomeric form depicted by Formula 1¹ also includes the tautomeric form depicted by Formula 1².

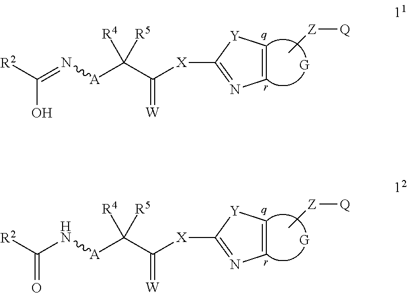

The compounds of the present invention include N-oxide derivatives of Formula 1. One skilled in the art will appreciate that not all nitrogen-containing heterocycles can form N-oxides since the nitrogen requires an available lone pair of electrons for oxidation to the oxide; one skilled in the art will recognize those nitrogen-containing heterocycles which can form N-oxides. One skilled in the art will also recognize that tertiary amines can form N-oxides. Synthetic methods for the preparation of N-oxides of heterocycles and tertiary amines are very well known by one skilled in the art including the oxidation of heterocycles and tertiary amines with peroxy acids such as peracetic and m-chloroperbenzoic acid (MCPBA), hydrogen peroxide, alkyl hydroperoxides such as tert-butyl hydroperoxide, sodium perborate, and dioxiranes such as dimethyldioxirane. These methods for the preparation of N-oxides have been extensively described and reviewed in the literature, see for example: T. L. Gilchrist in Comprehensive Organic Synthesis, vol. 7, pp 748-750, S. V. Ley, Ed., Pergamon Press; M. Tisler and B. Stanovnik in Comprehensive Heterocyclic Chemistry, vol. 3, pp 18-20, A. J. Boulton and A. McKillop, Eds., Pergamon Press; M. R. Grimmett and B. R. T. Keene in Advances in Heterocyclic Chemistry, vol. 43, pp 149-161, A. R. Katritzky, Ed., Academic Press; M. Tisler and B. Stanovnik in Advances in Heterocyclic Chemistry, vol. 9, pp 285-291, A. R. Katritzky and A. J. Boulton, Eds., Academic Press; and G. W. H. Cheeseman and E. S. G. Werstiuk in Advances in Heterocyclic Chemistry, vol. 22, pp 390-392, A. R. Katritzky and A. J. Boulton, Eds., Academic Press.

One skilled in the art recognizes that because in the environment and under physiological conditions salts of chemical compounds are in equilibrium with their corresponding nonsalt forms, salts share the biological utility of the nonsalt forms. When the compounds forming the present mixtures and compositions contain acidic or basic moieties, a wide variety of salts can be formed, and these salts are useful in the present mixtures and compositions for controlling plant diseases caused by fungal plant pathogens (i.e. are agriculturally suitable). When a compound contains a basic moiety such as an amine function, salts include acid-addition salts with inorganic or organic acids such as hydrobromic, hydrochloric, nitric, phosphoric, sulfuric, acetic, butyric, fumaric, lactic, maleic, malonic, oxalic, propionic, salicylic, tartaric, 4-toluenesulfonic or valeric acids. When a compound contains an acidic moiety such as a carboxylic acid or phenol, salts include those formed with organic or inorganic bases such as pyridine, triethylamine or ammonia, or amides, hydrides, hydroxides or carbonates of sodium, potassium, lithium, calcium, magnesium or barium.

Compounds selected from Formula 1, stereoisomers, N-oxides, and salts thereof, typically exist in more than one form, therefore Formula 1 includes all crystalline and non-crystalline forms of the compounds that Formula 1 represents. Non-crystalline forms include embodiments which are solids such as waxes and gums as well as embodiments which are liquids such as solutions and melts. Crystalline forms include embodiments which represent essentially a single crystal type and embodiments which represent a mixture of polymorphs (i.e. different crystalline types). The term “polymorph” refers to a particular crystalline form of a chemical compound that can crystallize in different crystalline forms, these forms having different arrangements and/or conformations of the molecules in the crystal lattice. Although polymorphs can have the same chemical composition, they can also differ in composition due to the presence or absence of co-crystallized water or other molecules, which can be weakly or strongly bound in the lattice. Polymorphs can differ in such chemical, physical and biological properties as crystal shape, density, hardness, color, chemical stability, melting point, hygroscopicity, suspensibility, dissolution rate and biological availability. One skilled in the art will appreciate that a polymorph of a compound represented by Formula 1 can exhibit beneficial effects (e.g., suitability for preparation of useful formulations, improved biological performance) relative to another polymorph or a mixture of polymorphs of the same compound represented by Formula 1. Preparation and isolation of a particular polymorph of a compound represented by Formula 1 can be achieved by methods known to those skilled in the art including, for example, crystallization using selected solvents and temperatures.

Embodiments of the present invention as described in the Summary of the Invention include those described below. In the following Embodiments, Formula 1 includes stereoisomers, tautomers, N-oxides, and salts thereof, and reference to “a compound of Formula 1” includes the definitions of substituents specified in the Summary of the Invention unless further defined in the Embodiments.

### Embodiment 1

A compound of Formula 1 wherein E is E-3.

### Embodiment 2

A compound of Formula 1 wherein E is E-1 or E-2.

### Embodiment 3

A compound of Formula 1 or Embodiment 2 wherein E is E-1.

### Embodiment 4

A compound of Formula 1 or Embodiment 2 wherein E is E-2.

### Embodiment 5

A compound of Formula 1 or any one of Embodiments 1 through 4 wherein X is X¹, X², X³, X⁴, X⁵ or X¹¹.

### Embodiment 6

A compound of Embodiment 5 wherein X is X¹, X² or X³.

### Embodiment 7

A compound of Embodiment 5 wherein X is X⁴, X⁵ or X.

### Embodiment 8

A compound of Embodiment 6 wherein X is X¹ or X².

### Embodiment 9

A compound of Embodiment 8 wherein X is X².

### Embodiment 10

A compound of Embodiment 8 wherein X is X¹.

### Embodiment 11

A compound of Formula 1 or any one of Embodiments 1 through 10 wherein Y is S.

### Embodiment 12

A compound of Formula 1 or any one of Embodiments 1 through 11 wherein G together with the two carbon atoms identified as “q” and “r” in Formula 1 forms a 5- to 6-membered ring containing ring members selected from carbon atoms and up to 2 heteroatoms independently selected from up to 1 O, up to 1 S and up to 2 N atoms, wherein up to 1 carbon atom ring member is selected from C(═O) and C(═NOH), the ring optionally substituted with up to 2 substituents independently selected from R⁸ on carbon atom ring members.

### Embodiment 13

A compound of Formula 1 or any one of Embodiments 1 through 12 wherein G together with the two carbon atoms indentified as “q” and “r” in Formula 1 forms a 5- to 6-membered ring selected from G-1 through G-34 in Exhibit 1.

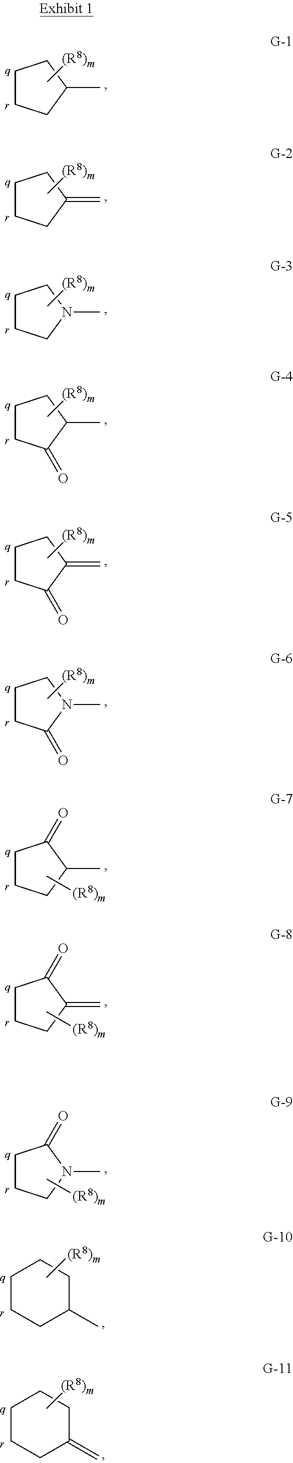

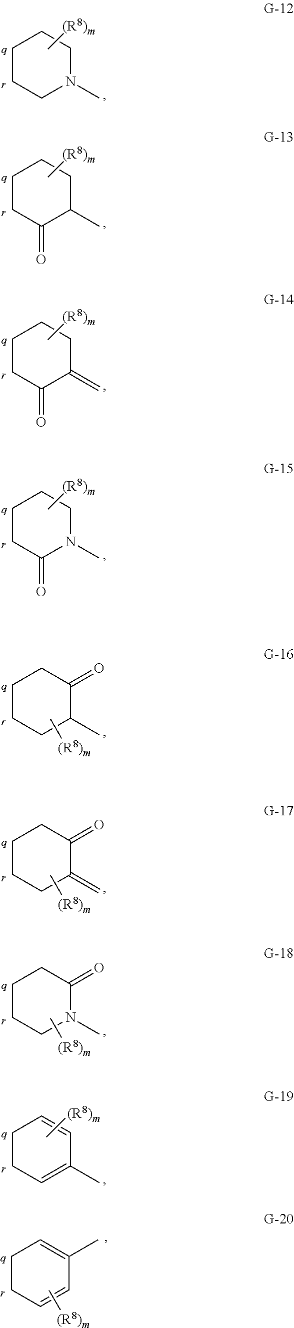

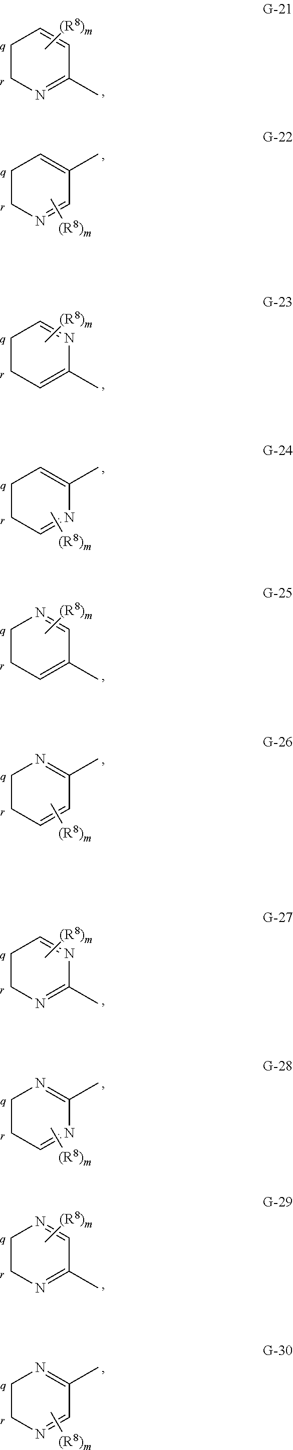

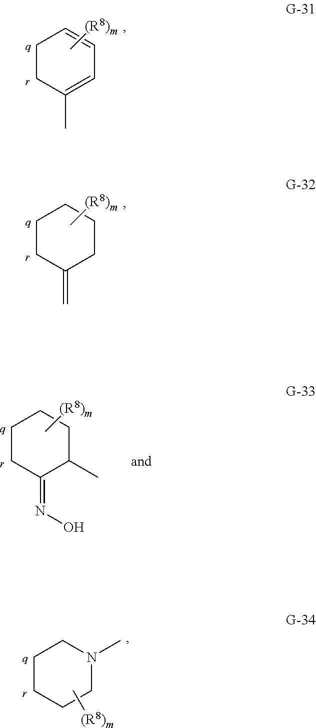

wherein the bond projecting to the right or down is connected to Z in Formula 1; and m is 0, 1 or 2.

### Embodiment 14

A compound of Embodiment 13 wherein G is selected from G-12, G-13, G-14, G-15, G-31, G-32 and G-33.

### Embodiment 15

A compound of Embodiment 14 wherein G is selected from G-12, G-13, G-14 and G-15.

### Embodiment 16

A compound of Embodiment 15 wherein G is selected from G-13 through G-15.

### Embodiment 17

A compound of Embodiment 16 wherein G is G-13.

### Embodiment 18

A compound of Embodiment 16 wherein G is G-15.

### Embodiment 19

A compound of Embodiment 13 wherein G is selected from G-1 through G-26.

### Embodiment 20

A compound of Embodiment 19 wherein G is selected from G-1 through G-20.

### Embodiment 21

A compound of Embodiment 20 wherein G is selected from G-4 through G-9 and G-13 through G-18.

### Embodiment 22

A compound of Embodiment 20 wherein G is selected from G-19 and G-20.

### Embodiment 23

A compound of Embodiment 20 wherein G is selected from G-4, G-6, G-7, G-9, G-13 and G-15.

### Embodiment 24

A compound of Embodiment 23 wherein G is G-4.

### Embodiment 25

A compound of Embodiment 23 wherein G is G-6.

### Embodiment 26

A compound of Embodiment 23 wherein G is G-7.

### Embodiment 27

A compound of Embodiment 23 wherein G is G-9.

### Embodiment 28

A compound of any one of Embodiments 13 through 27 wherein m is 0 or 1.

### Embodiment 29

A compound of Embodiment 28 wherein m is O.

### Embodiment 30

A compound of Formula 1 or any one of Embodiments 1 through 29 wherein Z is a saturated, partially unsaturated or fully unsaturated chain containing 1- to 3-atoms selected from up to 3 carbon, up to 1 O, up to 1 S and up to 2 N atoms, the chain optionally substituted with up to 1 substituent selected from R^{9a} on a carbon atom and R^{9b} on nitrogen atom.

### Embodiment 31

A compound of Embodiment 30 wherein Z is O, S, NH, CH₂, CH₂CH₂, CH₂CH₂CH₂, OCH₂, CH₂O, OCH₂CH₂, CH₂CH₂O, SCH₂, CH₂S, SCH₂CH₂, CH₂CH₂S, NHCH₂, CH₂NH, NHCH₂CH₂, CH₂CH₂NH, CH, CHCH₂, CHCH₂CH₂, NNH, NNHCH₂, NO or NOCH₂, each optionally substituted with up to 1 substituent selected from R^{9a} on a carbon atom and R^{9b} on a nitrogen atom.

### Embodiment 32

A compound of Embodiment 31 wherein Z is O, S, NH, CH₂, CH₂CH₂, OCH₂, CH₂O, SCH₂, CH₂S, NHCH₂, CH₂NH, CH or NOCH₂, each optionally substituted with up to 1 substituent selected from R^{9a} on a carbon atom and R^{9b} on a nitrogen atom.

### Embodiment 33

A compound of Embodiment 32 wherein Z is NH, CH₂, NHCH₂, CH or NOCH₂, each optionally substituted with up to 1 substituent selected from R^{9a} on a carbon atom and R^{9b} on a nitrogen atom.

### Embodiment 34

A compound of Embodiment 33 wherein Z is CH₂ or CH.

### Embodiment 35

A compound of Embodiment 34 wherein Z is CH₂.

### Embodiment 36

A compound of Formula 1 or any one of Embodiments 1 through 35 wherein Q is selected from Q-1 through Q-102 in Exhibit 2.

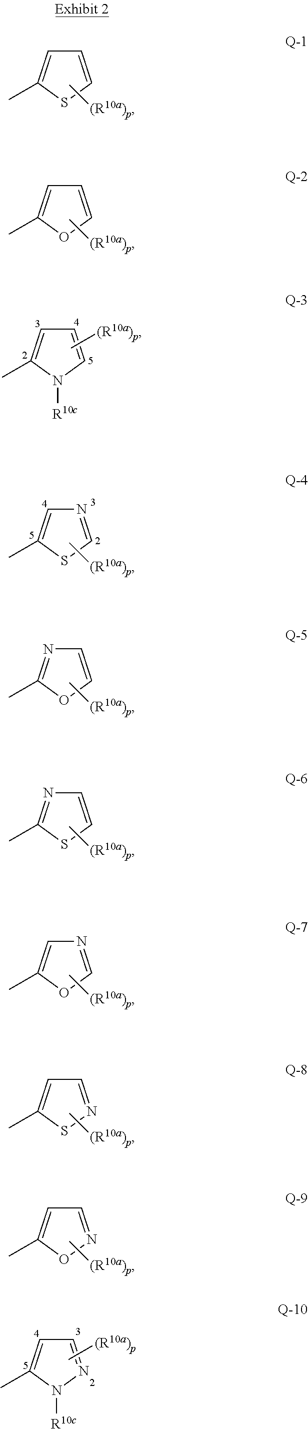

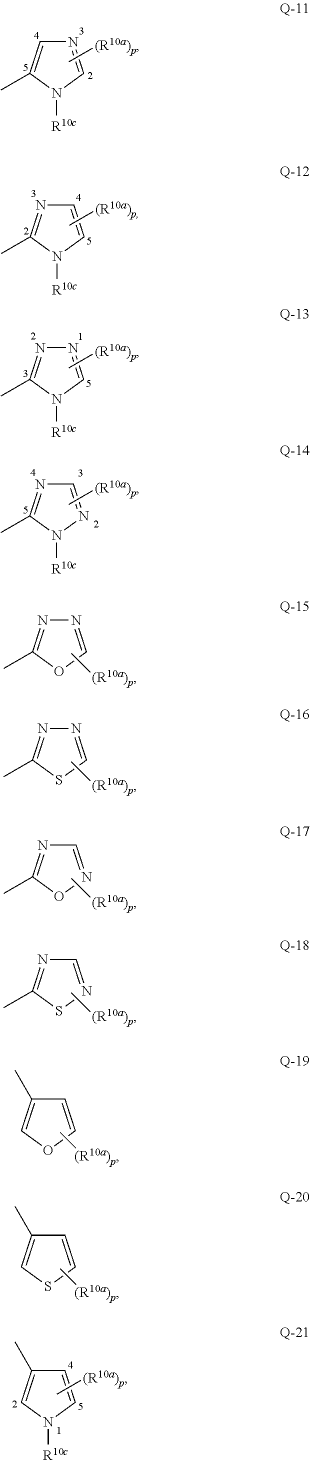

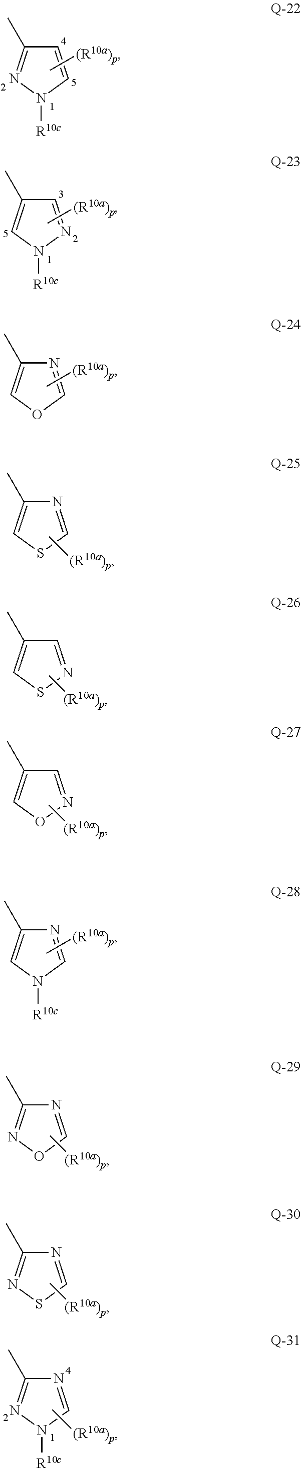

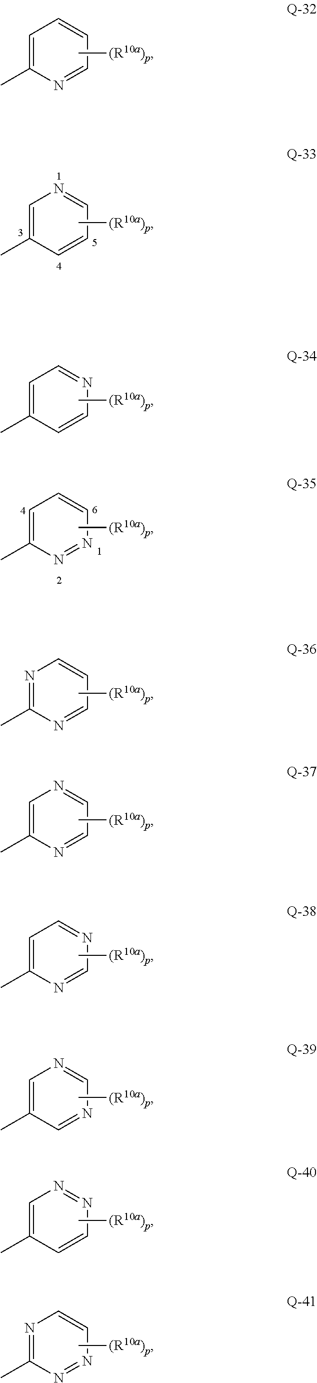

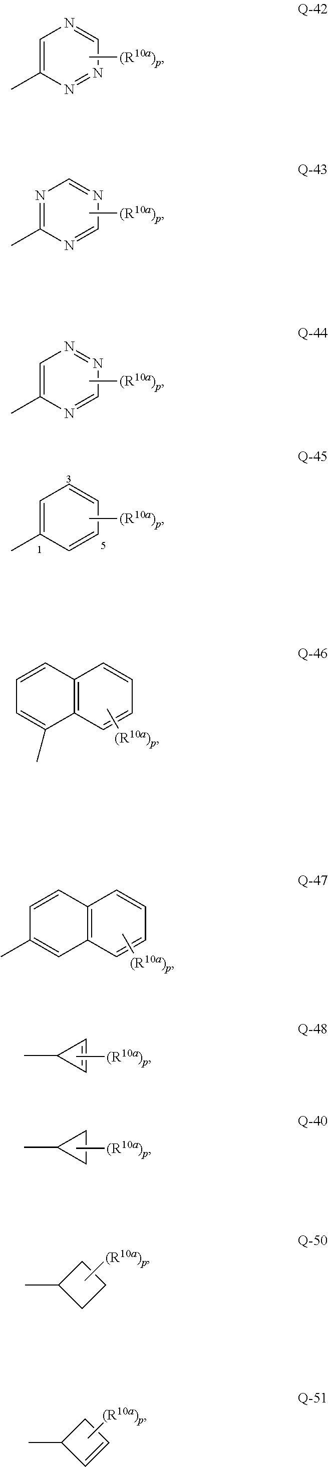

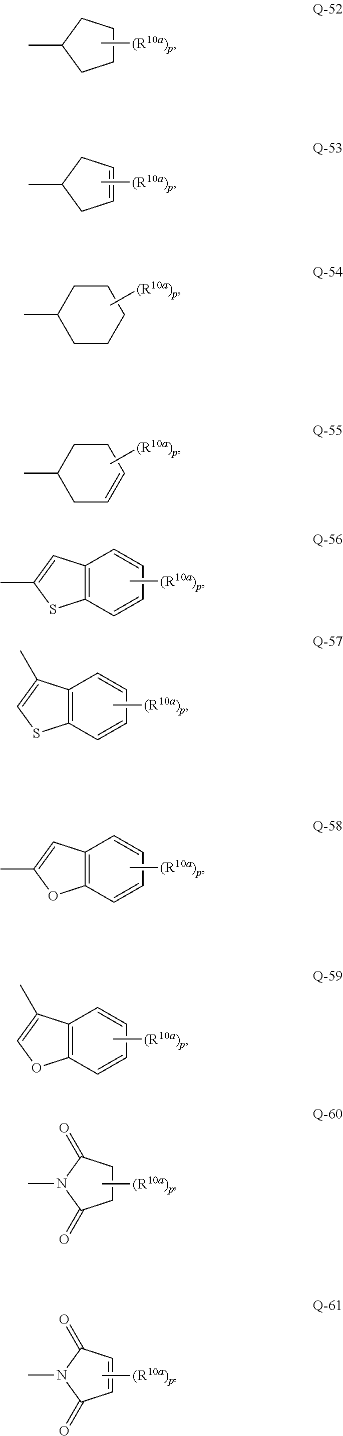

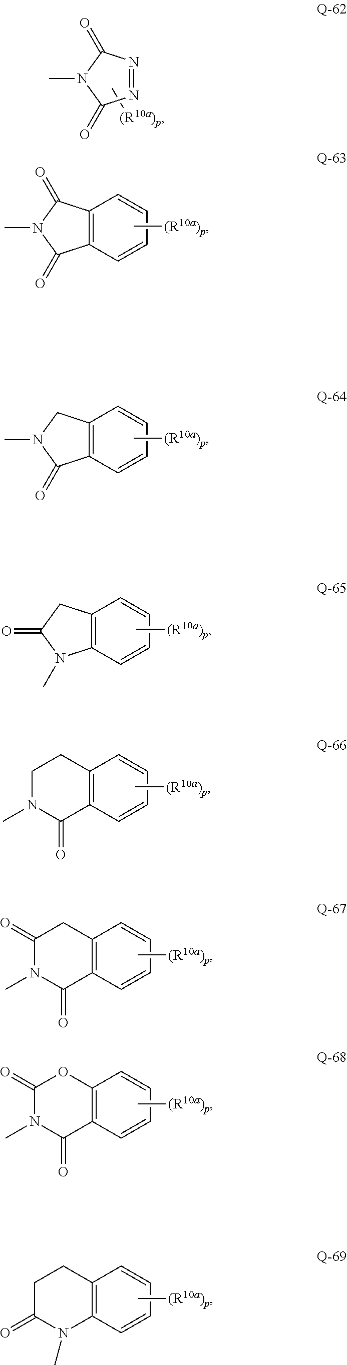

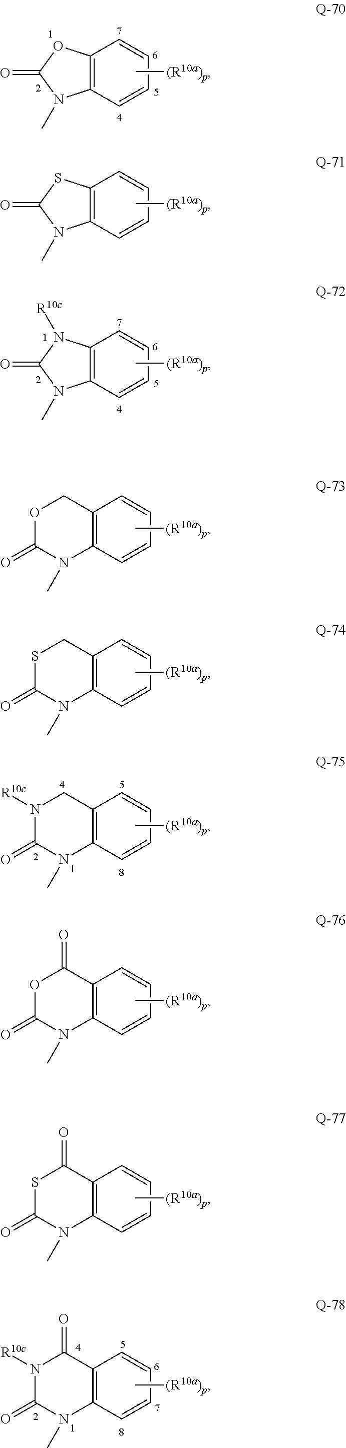

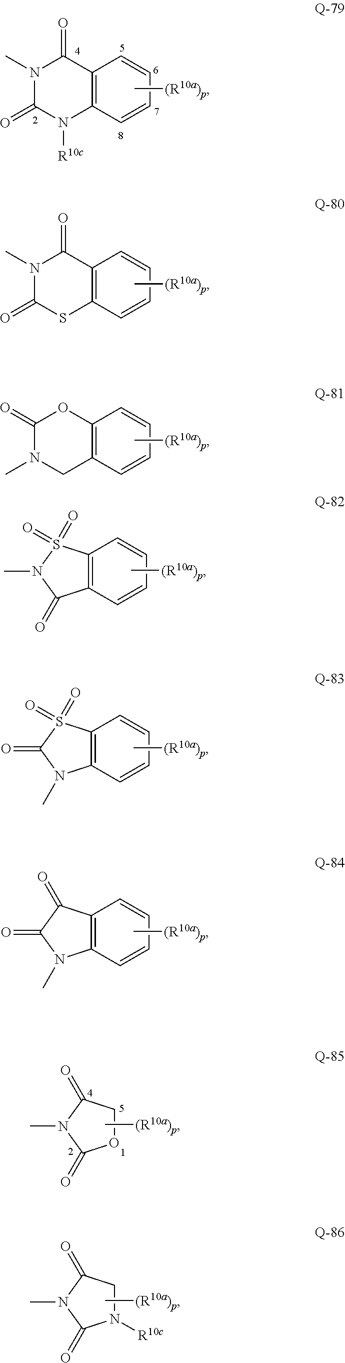

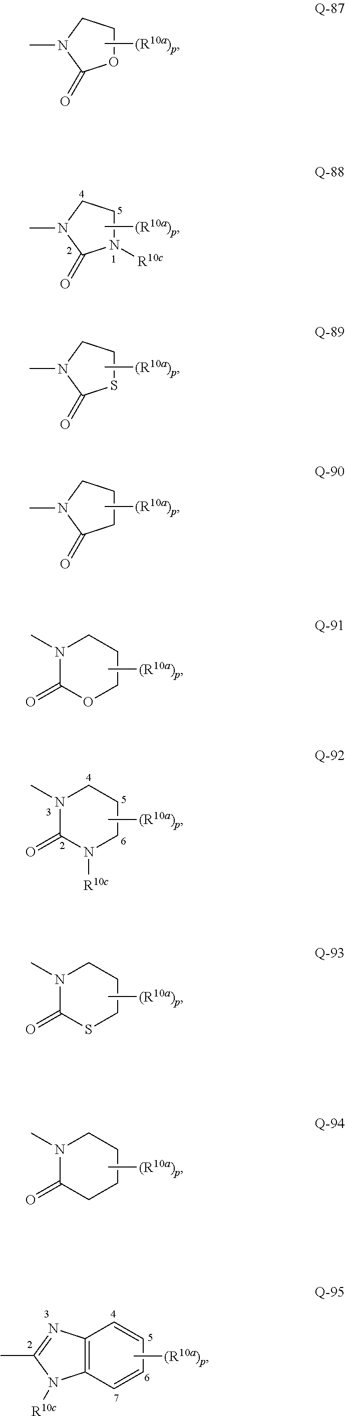

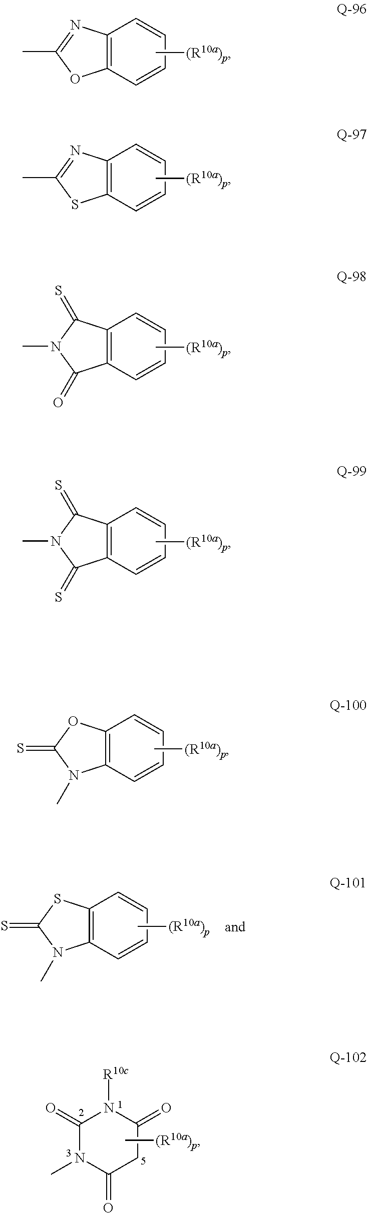

wherein the bond projecting to the left is connected to Z; R^{10c} is selected from H and R^{10b}; and p is 0, 1, 2 or 3.

### Embodiment 37

A compound of Embodiment 36 wherein Q is selected from Q-1, Q-20, Q-32, Q-33, Q-34, Q-45, Q-46, Q-47, Q-60 through Q-73, Q-76 through Q-79, Q-84 through Q-94 and Q-98 through Q-102.

### Embodiment 38

A compound of Embodiment 37 wherein Q is selected from Q-1, Q-45, Q-63, Q-64, Q-65, Q-68, Q-69, Q-70, Q-71, Q-72, Q-73, Q-76, Q-78, Q-79, Q-84, Q-85, Q-98, Q-99, Q-100, Q-101 and Q-102.

### Embodiment 39

A compound of Embodiment 38 wherein Q is selected from Q-45, Q-63, Q-64, Q-65, Q-68, Q-69, Q-70, Q-71, Q-72, Q-84 and Q-85.

### Embodiment 40

A compound of Embodiment 39 wherein Q is selected from Q-45, Q-63, Q-65, Q-70, Q-71, Q-72, Q-84 and Q-85.

### Embodiment 41

A compound of Embodiment 40 wherein Q is selected from Q-45, Q-63, Q-65, Q-70, Q-71, Q-72 and Q-84.

### Embodiment 42

A compound of Embodiment 41 wherein Q is selected from Q-45, Q-63, Q-70, Q-71, Q-72 and Q-84.

### Embodiment 43

A compound of Embodiment 42 wherein Q is Q-45.

### Embodiment 44

A compound of any one of Embodiments 36 through 43 wherein p is 0, 1 or 2.

### Embodiment 45

A compound of Embodiment 44 wherein p is O.

### Embodiment 46

A compound of Embodiment 44 wherein p is 2.

### Embodiment 47

A compound of Formula 1 or any one of Embodiments 1 through 46 wherein A is CH(R¹¹) or N(R¹²).

### Embodiment 48

A compound of Embodiment 47 wherein A is CH(R¹¹).

### Embodiment 48a

A compound of Embodiment 48 wherein A is CH₂.

### Embodiment 49

A compound of Embodiment 47 wherein A is N(R¹²).

### Embodiment 49a

A compound of Embodiment 49 wherein A is NH.

### Embodiment 50

A compound of Formula 1 or any one of Embodiments 1 or 49a wherein A¹ is O, S, C(R¹⁴)₂, N(R¹³) or —OC(R¹⁴)₂—, wherein the bond projecting to the left is connected to the nitrogen atom, and the bond projecting to the right is connected to the carbon atom in Formula 1.

### Embodiment 51

A compound of Embodiment 50 wherein A¹ is O, S or N(R¹³).

### Embodiment 52

A compound of Embodiment 51 wherein A¹ is O or N(R¹³).

### Embodiment 53

A compound of Formula 1 or any of Embodiments 1 through 52 wherein W is O.

### Embodiment 54

A compound of Formula 1 or any one of Embodiments 1 through 53 wherein W¹ is OR¹⁵, SR¹⁶ or NR¹⁷R¹⁸.

### Embodiment 55

A compound of Embodiment 54 wherein W¹ is OR¹⁵.

### Embodiment 56

A compound of Embodiment 54 wherein W¹ is SR¹⁶.

### Embodiment 57

A compound of Embodiment 54 wherein W¹ is NR¹⁷R¹⁸.

### Embodiment 58

A compound of Formula 1 or any one of Embodiments 1 through 57 wherein R¹ and R⁶ are each an optionally substituted phenyl, an optionally substituted naphthalenyl or an optionally substituted 5- to 6-membered heteroaromatic ring; or cyano, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₂-C₈ alkoxyalkyl, C₂-C₈ haloalkoxyalkyl, C₂-C₈ alkylthioalkyl, C₂-C₈ haloalkylthioalkyl, C₂-C₈ alkylsulfinylalkyl, C₂-C₈ alkylsulfonylalkyl, C₂-C₈ alkylaminoalkyl, C₂-C₈ haloalkylaminoalkyl, C₃-C₁₀ dialkylaminoalkyl, C₄-C₁₀ cycloalkylaminoalkyl, C₃-C₈ alkoxycarbonylalkyl, C₃-C₈ haloalkoxycarbonylalkyl, C₁-C₈ alkoxy, C₁-C₈ haloalkoxy, C₂-C₈ alkenyloxy, C₂-C₈ haloalkenyloxy, C₂-C₈ alkynyloxy, C₃-C₈ haloalkynyloxy, C₃-C₈ cycloalkoxy, C₃-C₈ halocycloalkoxy, C₄-C₁₀ cycloalkylalkoxy, C₂-C₈ alkoxyalkoxy, C₂-C₈ alkylcarbonyloxy, C₂-C₈ haloalkylcarbonyloxy, C₁-C₈ alkylthio, C₁-C₈ haloalkylthio, C₃-C₈ cycloalkylthio, C₁-C₈ alkylamino, C₂-C₈ dialkylamino, C₂-C₈ alkylcarbonylamino, C₃-C₁₀ trialkylsilyl, pyrrolidinyl, piperidinyl or morpholinyl.

### Embodiment 59

A compound of Embodiment 58 wherein R¹ and R⁶ are each cyano, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₂-C₈ alkoxyalkyl, C₂-C₅ haloalkoxyalkyl, C₂-C₈ alkylthioalkyl, C₂-C₅ haloalkylthioalkyl, C₂-C₈ alkylsulfinylalkyl, C₂-C₈ alkylsulfonylalkyl, C₂-C₈ alkylaminoalkyl, C₃-C₁₀ dialkylaminoalkyl, C₁-C₈ alkoxy, C₁-C₈ haloalkoxy, C₂-C₅ alkylcarbonyloxy, C₂-C₅ haloalkylcarbonyloxy, C₁-C₈ alkylthio, C₁-C₈ alkylamino, C₂-C₈ dialkylamino, C₂-C₈ alkylcarbonylamino, C₃-C₁₀ trialkylsilyl, pyrrolidinyl, piperidinyl or morpholinyl.

### Embodiment 60

A compound of Embodiment 59 wherein R¹ and R⁶ are each C₂-C₅ alkyl, C₂-C₅ haloalkyl, C₂-C₅ alkenyl, C₂-C₅ haloalkenyl, C₂-C₅ alkoxyalkyl, C₂-C₅ haloalkoxyalkyl, C₂-C₅ alkylthioalkyl, C₂-C₅ haloalkylthioalkyl, C₂-C₅ alkylaminoalkyl, C₂-C₅ alkoxy, C₂-C₅ haloalkoxy, C₂-C₅ alkylcarbonyloxy, C₂-C₅ haloalkylcarbonyloxy, C₂-C₅ alkylthio, C₂-C₅ alkylamino or C₂-C₅ alkylcarbonylamino.

### Embodiment 61

A compound of Embodiment 60 wherein R¹ and R⁶ are each C₃-C₅ alkyl, C₃-C₅ haloalkyl, C₃-C₅ alkenyl, C₃-C₅ haloalkenyl, C₂-C₄ alkoxyalkyl, C₂-C₄ haloalkoxyalkyl, C₂-C₄ alkylthioalkyl, C₂-C₄ haloalkylthioalkyl, C₂-C₄ alkoxy, C₂-C₄ haloalkoxy, C₂-C₃ alkylcarbonyloxy or C₂-C₃ haloalkylcarbonyloxy.

### Embodiment 62

A compound of Embodiment 61 wherein R¹ and R⁶ are each C₃-C₅ haloalkyl, C₃-C₅ haloalkenyl, C₃-C₅ haloalkoxyalkyl, C₃-C₅ haloalkylthioalkyl, C₂-C₄ haloalkoxy or C₂-C₃ haloalkylcarbonyloxy.

### Embodiment 63

A compound of Embodiment 62 wherein R¹ and R⁶ are each C₄ haloalkyl, C₄ haloalkenyl, C₃ haloalkoxyalkyl or C₃ haloalkoxy.

### Embodiment 64

A compound of Formula 1 or any one of Embodiments 1 through 63 wherein when R¹ and R⁶ are each optionally substituted phenyl, optionally substituted naphthalenyl or an optionally substituted 5- or 6-membered heteroaromatic ring, then the optional substituents on the phenyl, naphthalenyl or 5or 6-membered heteroaromatic ring are independently selected from R^{23a} on carbon atom ring members and R^{23b} on nitrogen atom ring members;
each R^{23a} is independently amino, cyano, halogen, hydroxy, nitro, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₄ hydroxyalkyl, C₃-C₆ cycloalkyl, C₃-C₆ halocycloalkyl, C₄-C₁₀ cycloalkylalkyl, C₄-C₁₀ alkylcycloalkyl, C₅-C₁₀ alkylcycloalkylalkyl, C₂-C₄ alkoxyalkyl, C₁-C₄ alkoxy, C₁-C₄ haloalkoxy, C₂-C₆ alkylcarbonyloxy, C₁-C₄ alkylthio, C₁-C₄ haloalkylthio, C₂-C₆ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; and
each R^{23b} is independently C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₃-C₆ halocycloalkyl or C₂-C₄ alkoxyalkyl.

### Embodiment 65

A compound of Formula 1 or any one of Embodiments 1 through 64 wherein R¹ and R⁶ are each a ring selected from U-1 through U-50 in Exhibit 3.

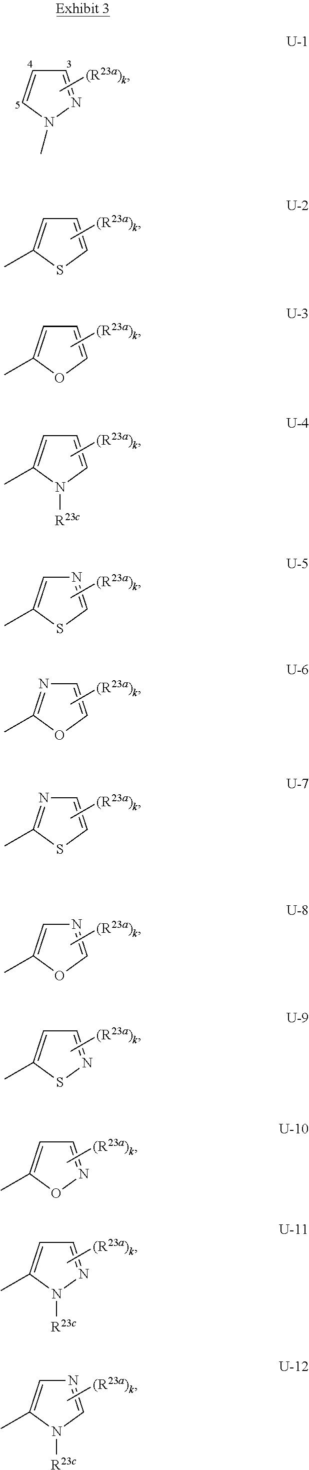

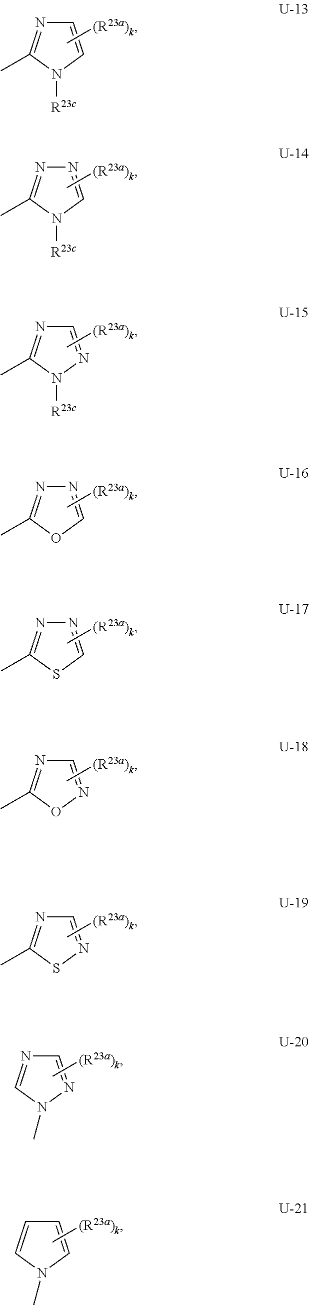

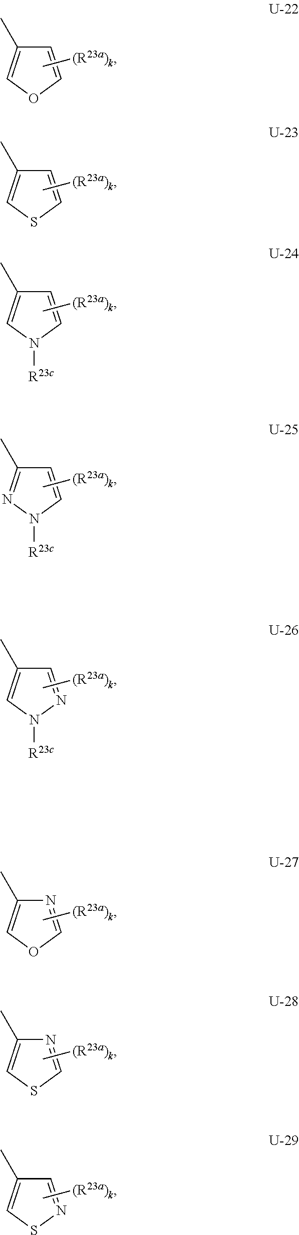

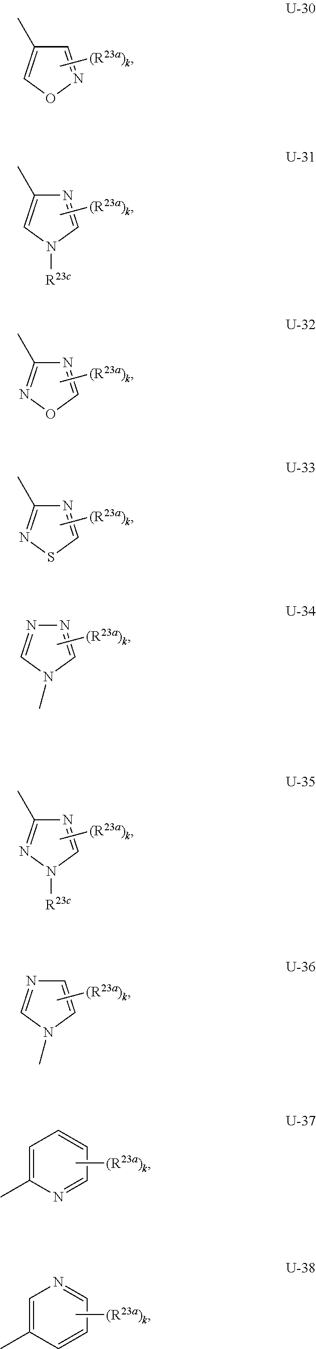

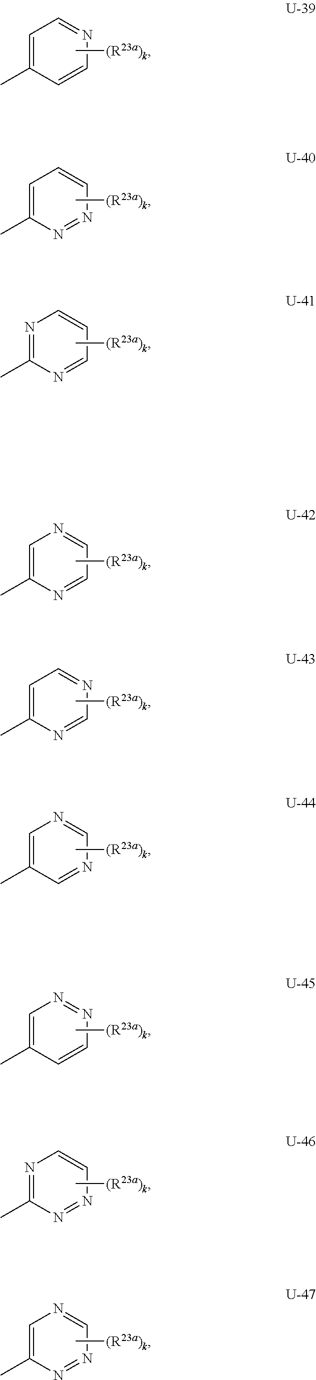

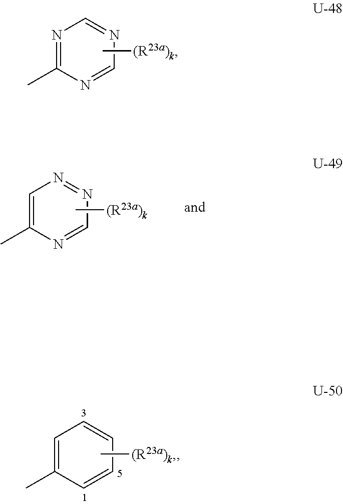

wherein the bond projecting to the left is connected to Formula 1; R^{23c} is selected from H and R^{23b}; and k is 0, 1, 2 or 3.

### Embodiment 66

A compound of Embodiment 65 wherein R¹ and R⁶ are each selected from U-1 through U-5, U-8, U-11, U-13, U-15, U-20 through U-28, U-31, U-36 through U-39 and U-50.

### Embodiment 67

A compound of Embodiment 66 wherein R¹ and R⁶ are each selected from U-1 through U-3, U-5, U-8, U-11, U-13, U-20, U-22, U-23, U-25 through U-28, U-36 through U-39 and U-50.

### Embodiment 68

A compound of Embodiment 67 wherein R¹ and R⁶ are each selected from U-1 through U-3, U-11, U-13, U-20, U-22, U-23, U-36 through U-39 and U-50.

### Embodiment 69

A compound of Embodiment 68 wherein R¹ and R⁶ are each selected from U-1, U-20 and U-50.

### Embodiment 70

A compound of Embodiment 69 wherein R¹ is selected from U-1, U-20 and U-50.

### Embodiment 71

A compound of Embodiment 69 wherein R¹ and R⁶ are each U-1.

### Embodiment 72

A compound of Embodiment 71 wherein R¹ is U-1.

### Embodiment 73

A compound of Embodiment 69 wherein R¹ and R⁶ are each U-20.

### Embodiment 74

A compound of Embodiment 69 wherein R¹ and R⁶ are each are U-50.

### Embodiment 75

A compound of any one of Embodiments 65 through 74 wherein k is 0, 1 or 2.

### Embodiment 76

A compound of Embodiment 75 wherein k is 2.

### Embodiment 77

compound of any one of Embodiments 64 through 76 wherein each R^{23a} is independently halogen, C₁-C₆ alkyl, C₁-C₆ haloalkyl or C₂-C₄ alkoxyalkyl.

### Embodiment 78

A compound of Embodiment 77 wherein each R^{23a} is independently halogen, C₁-C₃ alkyl, C₁-C₃ haloalkyl or C₂-C₃ alkoxyalkyl.

### Embodiment 79

A compound of Embodiment 78 wherein each R^{23a} is independently halogen, methyl or C₁-C₂ haloalkyl.

### Embodiment 80

A compound of Embodiment 79 wherein each R^{23a} is independently halogen, methyl or CF₃.

### Embodiment 81

A compound of compound of any one of Embodiments 64 through 80 wherein each R^{23b} is independently C₁-C₃ alkyl.

### Embodiment 82

A compound of Formula 1 or any one of Embodiments 1 through 81 wherein R² when taken alone (i.e. not taken together with R³) is H, cyano, C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkoxyalkyl, C₂-C₄ alkylthioalkyl, C₁-C₄ alkoxy, C₁-C₄ haloalkoxy, C₂-C₄ alkenyloxy, C₂-C₄ haloalkenyloxy, C₂-C₄ alkynyloxy, C₃-C₄ haloalkynyloxy, C₂-C₄ alkoxyalkoxy, C₁-C₄ alkylthio, C₁-C₄ haloalkylthio, C₁-C₄ alkylamino, C₁-C₄ haloalkylamino C₂-C₄ dialkylamino, C₂-C₄ halodialkylamino, C₂-C₄ alkylcarbonyl, C₂-C₄ haloalkylcarbonyl or C₂-C₄ alkoxycarbonyl.

### Embodiment 83

A compound of Embodiment 82 wherein R² when taken alone is H, cyano, C₁-C₃ alkyl, C₁-C₃ haloalkyl, C₂-C₃ alkenyl, C₂-C₃ haloalkenyl, C₂-C₃ alkynyl, C₂-C₃ haloalkynyl, C₁-C₃ alkoxy or C₁-C₃ haloalkoxy.

### Embodiment 84

A compound of Embodiment 83 wherein R² when taken alone is H, C₁-C₃ alkyl or C₁-C₃ haloalkyl.

### Embodiment 85

A compound of Embodiment 84 wherein R² when taken alone is H, C₁-C₃ alkyl or C₁-C₃ fluoroalkyl.

### Embodiment 86

A compound of Embodiment 85 wherein R² when taken alone is methyl, trifluoromethyl or CF₃CH₂—.

### Embodiment 87

A compound of Formula 1 or any one of Embodiments 1 through 86 wherein R² is taken alone.

### Embodiment 88

A compound of Formula 1 or any one of Embodiments 1 through 87 wherein R³ when taken alone (i.e. not taken together with R² or R¹³) is H, C₁-C₃ alkyl, C₁-C₃ haloalkyl or C₁-C₃ alkoxy.

### Embodiment 89

A compound of Embodiment 88 wherein R³ when taken alone is H, C₁-C₃ alkyl or C₁-C₃ haloalkyl.

### Embodiment 90

A compound of Embodiment 89 wherein R³ when taken alone is H, C₁-C₂ alkyl or C₁-C₃ fluoroalkyl.

### Embodiment 91

A compound of Embodiment 90 wherein R³ when taken alone is H, methyl or trifluoromethyl.

### Embodiment 92

A compound of Formula 1 or any one of Embodiments 1 through 91 wherein R³ is taken alone.

### Embodiment 93

A compound of Formula 1 or any one of Embodiments 1 through 92 wherein when R² and R³ are taken together with the carbon atom to which they are attached to form a ring, said ring has 3- to 6-members selected from carbon atoms and up to 2 heteroatoms independently selected from up to 2 O, up to 2 S and up to 2 N, wherein up to 1 carbon atom ring member is selected from C(═O) and C(═S), the ring optionally substituted with up to 3 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members.

### Embodiment 94

A compound of Formula 1 or any one of Embodiments 1 through 93 wherein R⁴ is optionally substituted phenyl, optionally substituted naphthalenyl or an optionally substituted 5- to 6-membered heteroaromatic ring; or H, cyano, hydroxy, C₁-C₃ alkyl, C₁-C₃ haloalkyl, C₂-C₃ alkenyl, C₂-C₃ haloalkenyl, C₂-C₃ alkynyl, C₂-C₃ haloalkynyl, C₁-C₃ alkoxy, C₁-C₃ haloalkoxy, C₂-C₃ alkylcarbonyloxy, C₂-C₃ haloalkylcarbonyloxy, C₁-C₃ alkylthio, C₁-C₃ haloalkylthio, C₂-C₃ alkylcarbonyl or C₂-C₃ haloalkylcarbonyl.

### Embodiment 95

A compound of Embodiment 94 wherein R⁴ is H, cyano, hydroxy, C₁-C₃ alkyl, C₁-C₃ haloalkyl, C₂-C₃ alkenyl, C₂-C₃ haloalkenyl, C₂-C₃ alkynyl, C₂-C₃ haloalkynyl, C₁-C₃ alkoxy, C₁-C₃ haloalkoxy, C₂-C₃ alkylcarbonyloxy, C₂-C₃ haloalkylcarbonyloxy, C₁-C₃ alkylthio, C₁-C₃ haloalkylthio, C₂-C₃ alkylcarbonyl or C₂-C₃ haloalkylcarbonyl.

### Embodiment 96

A compound of Embodiment 95 wherein R⁴ is H, cyano, hydroxy, C₁-C₃ alkyl, C₁-C₃ haloalkyl, C₁-C₃ alkoxy, C₁-C₃ haloalkoxy, C₂-C₃ alkylcarbonyloxy, C₂-C₃ haloalkylcarbonyloxy, C₁-C₃ alkylthio or C₁-C₃ haloalkylthio.

### Embodiment 97

A compound of Embodiment 96 wherein R⁴ is H, cyano, methyl, CH₃O— or CH₃C(═O)O—.

### Embodiment 98

A compound of Embodiment 97 wherein R⁴ is H or methyl.

### Embodiment 99

A compound of Embodiment 98 wherein R⁴ is H.

### Embodiment 100

A compound of Formula 1 or any one of Embodiments 1 through 99 wherein when R⁴ is optionally substituted phenyl, optionally substituted naphthalenyl or an optionally substituted 5- to 6-membered heteroaromatic ring, then the optional substituents on the phenyl, naphthalenyl or 5- to 6-membered heteroaromatic ring are independently selected from R^{24a} on carbon atom ring members and R^{24b} on nitrogen atom ring members;
each R^{24a} is independently amino, cyano, halogen, hydroxy, nitro, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₄ hydroxyalkyl, C₃-C₆ cycloalkyl, C₃-C₆ halocycloalkyl, C₄-C₁₀ cycloalkylalkyl, C₄-C₁₀ alkylcycloalkyl, C₅-C₁₀ alkylcycloalkylalkyl, C₂-C₄ alkoxyalkyl, C₁-C₄ alkoxy, C₁-C₄ haloalkoxy, C₂-C₆ alkylcarbonyloxy, C₁-C₄ alkylthio, C₁-C₄ haloalkylthio, C₂-C₆ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; and
each R^{24b} is independently C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₃-C₆ halocycloalkyl or C₂-C₄ alkoxyalkyl.

### Embodiment 101

A compound of Formula 1 or any one of Embodiments 1 through 100 wherein when R⁴ is optionally substituted phenyl, optionally substituted naphthalenyl or an optionally substituted 5- to 6-membered heteroaromatic ring, then R⁴ is other than optionally substituted naphthalenyl.

### Embodiment 102

A compound of Formula 1 or any one of Embodiments 1 through 101 wherein when R⁴ is optionally substituted phenyl or an optionally substituted 5- to 6-membered heteroaromatic ring, then R⁴ is a ring selected from L-1 through L-11 in Exhibit 4.

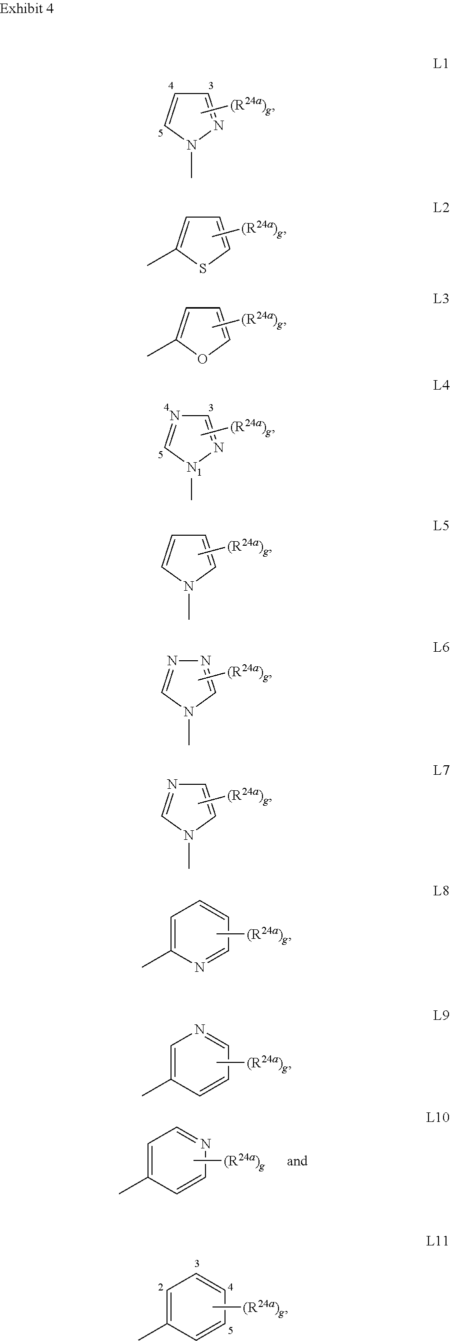

wherein g is 0, 1, 2 or 3.

### Embodiment 103

A compound of any one of Embodiments 100 through 102 wherein each R^{24a} is independently halogen, C₁-C₂ alkyl, C₁-C₂ haloalkyl or C₁-C₂ alkoxy.

### Embodiment 104

A compound of Embodiment 103 wherein each R^{24a} is independently Cl, Br, I, C₁-C₂ alkyl, trifluoromethyl or methoxy.

### Embodiment 105

A compound of Embodiment 104 wherein each R^{24a} is independently Cl, Br, C₁-C₂ alkyl or trifluoromethyl.

### Embodiment 106

A compound of Formula 1 or any one of Embodiments 1 through 105 wherein R⁵ is H or C₁-C₂ alkyl.

### Embodiment 107

A compound of Embodiment 106 wherein R⁵ is H.

### Embodiment 108

A compound of Formula 1 or any one of Embodiments 1 through 107 wherein each R^{7a} is independently cyano, halogen, hydroxy, C₁-C₂ alkyl, C₁-C₂ haloalkyl or C₁-C₂ alkoxy.

### Embodiment 109

A compound of Embodiment 108 wherein each R^{7a} is independently cyano, hydroxy methyl or methoxy.

### Embodiment 110

A compound of Embodiment 109 wherein each R^{7a} is methyl.

### Embodiment 111

A compound of Formula 1 or any one of Embodiments 1 through 110 wherein n is 0 or 1.

### Embodiment 112

A compound of Embodiment 111 wherein n is O.

### Embodiment 113

A compound of Formula 1 or any one of Embodiments 1 through 112 wherein R^{7b} is H or C₁-C₂ alkyl.

### Embodiment 114

A compound of Embodiment 113 wherein R^{7b} is H.

### Embodiment 115

A compound of Formula 1 or any one of Embodiments 1 through 115 wherein each R⁸ is independently halogen, hydroxy or methyl.

### Embodiment 116

A compound of Embodiment 115 wherein each R⁸ is methyl.

### Embodiment 117

A compound of Formula 1 or any one of Embodiments 1 through 116 wherein each R^{9a} is independently halogen, C₁-C₄ alkyl or C₁-C₄ alkoxy.

### Embodiment 118

A compound of Embodiment 117 wherein each R^{9a} is methyl.

### Embodiment 119

A compound of Formula 1 or any one of Embodiments 1 through 118 wherein each R^{9b} is independently C₁-C₄ alkyl.

### Embodiment 120

A compound of Embodiment 119 wherein each R^{9b} is methyl.

### Embodiment 121

A compound of Formula 1 or any one of Embodiments 1 through 120 wherein each R^{10a} is independently amino, halogen, cyano, C₁-C₆ alkyl, C₁-C₆ haloalkyl, C₂-C₆ alkenyl, C₂-C₆ alkynyl, C₃-C₆ cycloalkyl, C₃-C₆ halocycloalkyl, C₄-C₁₀ cycloalkylalkyl, C₂-C₄ alkoxyalkyl, C₁-C₄ alkoxy, C₁-C₄ haloalkoxy, C₂-C₆ alkylcarbonyloxy, C₁-C₄ alkylthio, C₁-C₄ alkylsulfonyl, C₁-C₄ alkylamino, C₂-C₈ dialkylamino, C₂-C₄ alkylcarbonyl, C₂-C₆ alkoxycarbonyl, C₂-C₆ alkylaminocarbonyl or C₃-C₈ dialkylaminocarbonyl; or phenyl optionally substituted with up to 3 substituents independently selected from halogen, C₁-C₂ alkyl, C₁-C₂ haloalkyl and C₁-C₂ alkoxy.

### Embodiment 122

A compound of Embodiment 121 wherein each R^{10a} is independently halogen, C₁-C₆ alkyl, C₁-C₆ haloalkyl or C₁-C₆ alkoxy.

### Embodiment 122a

A compound of Embodiment 122 wherein each R^{10a} is independently halogen, C₁-C₂ alkyl, C₁-C₂ haloalkyl or C₁-C₂ alkoxy.

### Embodiment 123

A compound of Embodiment 122a wherein each R^{10a} is independently F or CH₃.

### Embodiment 124

A compound of Formula 1 or any one of Embodiments 1 through 123 wherein R^{10c} is C₁-C₃ alkyl, C₃-C₆ cycloalkyl, C₂-C₃ alkylcarbonyl or C₂-C₃ alkoxycarbonyl.

### Embodiment 125

A compound of Embodiment 124 wherein each R^{10c} is methyl, CH₃C(═O) or CH₃OC(═O).

### Embodiment 126

A compound of Formula 1 or Embodiments 1 through 125 wherein R¹¹ is H, cyano, halogen, hydroxy, —CH(═O), C₁-C₄ alkyl, C₁-C₄ haloalkyl, C₁-C₄ alkoxy or C₂-C₅ alkoxycarbonyl.

### Embodiment 127

A compound of Embodiment 126 wherein R¹¹ is H, cyano, halogen, hydroxy, methyl or methoxy.

### Embodiment 128

A compound of Embodiment 127 wherein R¹¹ is H.

### Embodiment 129

A compound of Formula 1 or Embodiments 1 through 128 wherein R¹² is H, methyl, CH₃C(═O) or CH₃OC(═O).

### Embodiment 130

A compound of Embodiment 129 wherein R¹² is H.

### Embodiment 131

A compound of Formula 1 or any one of Embodiments 1 through 130 wherein R¹³ when taken alone (i.e. not taken together with R³) is H, C₁-C₂ alkyl, C₁-C₂ haloalkyl, CH₃C(═O), CF₃C(═O) or CH₃OC(═O).

### Embodiment 132

A compound of Embodiment 131 wherein R¹³ when taken alone is H or C₁-C₂ alkyl.

### Embodiment 133

A compound of Embodiment 132 wherein R¹³ when taken alone is H or methyl.

### Embodiment 134

A compound of Formula 1 or any one of Embodiments 1 through 133 wherein R¹³ is taken alone.

### Embodiment 135

A compound of Formula 1 or any one of Embodiments 1 through 134 wherein each R¹⁴ is independently H or methyl.

### Embodiment 136

A compound of Embodiment 135 wherein each R¹⁴ is H.

### Embodiment 137

A compound of Formula 1 or any one of Embodiments 1 through 136 wherein R¹⁵ and R¹⁶ are each C₁-C₆ alkyl, C₁-C₄ haloalkyl, C₃-C₄ alkenyl, C₃-C₆ haloalkenyl, C₃-C₄ alkynyl, C₃-C₆ haloalkynyl, C₃-C₆ cycloalkyl or C₂-C₆ alkoxyalkyl.

### Embodiment 138

A compound of Embodiment 137 wherein R¹⁵ and R¹⁶ are each C₁-C₆ alkyl, C₁-C₄ haloalkyl, C₃-C₄ alkenyl or C₃-C₄ alkynyl.

### Embodiment 139

A compound of Embodiment 138 wherein R¹⁵ and R¹⁶ are each is C₁-C₄ alkyl.

### Embodiment 140

A compound of Formula 1 or any one of Embodiments 1 through 139 wherein R¹⁷ when taken alone (i.e. not taken together with R¹⁸) is H, amino, cyano, hydroxy or C₁-C₆ alkyl.

### Embodiment 141

A compound of Formula 1 or any one of Embodiments 1 through 140 wherein R¹⁸ when taken alone (i.e. not taken together with R¹⁷) is H or C₁-C₆ alkyl.

### Embodiment 142

A compound of Formula 1 or any one of Embodiments 1 through 141 wherein when R¹⁷ and R¹⁸ are taken together, then R¹⁷ and R¹⁸ are taken together as —(CH₂)₄— or —(CH₂)₂O(CH₂)₂—.

### Embodiment 143

A compound of Embodiment 142 wherein when R¹⁷ and R¹⁸ are taken together, then R¹⁷ and R¹⁸ are taken together as —(CH₂)₄—.

### Embodiment 144

A compound of Formula 1 or any one of Embodiments 1 through 143 wherein s and f are both 0.

Embodiments of this invention, including Embodiments 1-144 above as well as any other embodiments described herein, including Embodiments A1-A3 below, can be combined in any manner, and the descriptions of variables in the embodiments pertain not only to the compounds of Formula 1 but also to the starting compounds and intermediate compounds useful for preparing the compounds of Formula 1 unless further defined in the Embodiments. In addition, embodiments of this invention, including Embodiments 1-144 above as well as any other embodiments described herein, and any combination thereof, pertain to the compositions and methods of the present invention. Combinations of Embodiments 1-144 are illustrated by:

### Embodiment A1

A compound of Formula 1 wherein
E is E-1 or E-2;
X is X¹ or X²;
Y is S;
G is selected from G-12, G-13, G-14, G-15, G-31, G-32 and G-33 (as shown in Exhibit 1), wherein the bond projecting to the right or down is connected to Z in Formula 1;
m is 0, 1 or 2;
Z is NH, CH₂, NHCH₂, CH or NOCH₂, each optionally substituted with up to 1 substituent selected from R^{9a} on a carbon atom and R^{9b} on a nitrogen atom;
Q is selected from Q-45, Q-63, Q-65, Q-70, Q-71, Q-72 and Q-84 (as shown in Exhibit 2), wherein the bond projecting to the left is connected to Z;
p is 0, 1 or 2;
R^{10c} is selected from H and R^{10b};
A is CH(R¹¹) or N(R¹²);
A¹ is O or N(R¹³);
W is O;
R¹ is selected from U-1, U-20 and U-50 (as shown in Exhibit 3), wherein the bond projecting to the left is connected to Formula 1;
k is 0, 1 or 2;
each R^{23a} is independently halogen, C₁-C₃ alkyl, C₁-C₃ haloalkyl or C₂-C₃ alkoxyalkyl;
R² is H, C₁-C₃ alkyl or C₁-C₃ haloalkyl;
R³ is H, C₁-C₃ alkyl or C₁-C₃ haloalkyl;
R⁴ is H or methyl;
R⁵ is H or C₁-C₂ alkyl;
each R^{7a} is independently cyano, halogen, hydroxy, C₁-C₂ alkyl, C₁-C₂ haloalkyl or C₁-C₂ alkoxy;
R⁸ is independently halogen, hydroxy or methyl;
each R^{9a} is halogen, C₁-C₄ alkyl or C₁-C₄ alkoxy;
each R^{9b} is C₁-C₄ alkyl;
each R^{10a} is independently halogen, C₁-C₆ alkyl, C₁-C₆ haloalkyl or C₁-C₆ alkoxy;
R¹¹ is H, cyano, halogen, hydroxy, —CH(═O), C₁-C₄ alkyl, C₁-C₄ haloalkyl, C₁-C₄ alkoxy or C₂-C₅ alkoxycarbonyl;
R¹² is H, methyl, CH₃C(═O) or CH₃OC(═O); and
R¹³ is H or methyl.

### Embodiment A2

A compound of Embodiment A¹ wherein
E is E-1;
G is selected from G-12, G-13, G-14 and G-15;
m is 0;
Q is Q-45;
A is CH(R¹¹);
R¹ is U-1;
each R^{23a} is independently halogen, methyl or C₁-C₂ haloalkyl;
each R^{9a} is methyl;
each R^{9b} is methyl;
each R^{10a} is independently halogen, C₁-C₂ alkyl, C₁-C₂ haloalkyl or C₁-C₂ alkoxy;
R¹¹ is H; and
n is 0.

### Embodiment A3

A compound of Embodiment A2 wherein
X is X-1;
G is selected from G-13, G-14 and G-15; and
Z is CH₂ or CH.

Specific embodiments include compounds of Formula 1 selected from the group consisting of:
6,7-dihydro-2-[1-[2-[5-methyl-3-(trifluoromethyl)-1H-pyrazol-1-yl]acetyl]-4-piperidinyl]-5-(phenylmethyl)thiazol[4,5-c]pyridin-4(5H)-one;
5-[(2,6-difluorophenyl)methyl]-6,7-dihydro-2-[1-[2-[5-methyl-3-(trifluoromethyl)-1H-pyrazol-1-yl]acetyl]-4-piperidinyl]-4(5H)-benzonthiazolone; and
5-[(2,6-difluorophenyl)methyl]-6,7-dihydro-2-[1-[2-[5-methyl-3-(trifluoromethylene)-1H-pyrazol-1-yl]acetyl]-4-piperidinyl]-4(5H)-benzothiazolone.

This invention provides a fungicidal composition comprising a compound selected from Formula 1 (including all geometric and stereoisomers, tautomers, N-oxides, and salts thereof) and at least one other fungicide. Of note as embodiments of such compositions are compositions comprising a compound corresponding to any of the compound embodiments described above.

This invention provides a fungicidal composition comprising a fungicidally effective amount of a compound selected from Formula 1 (including all geometric and stereoisomers, tautomers, N-oxides, and salts thereof) (i.e. in a fungicidally effective amount), and at least one additional component selected from the group consisting of surfactants, solid diluents and liquid diluents. Of note as embodiments of such compositions are compositions comprising a compound corresponding to any of the compound embodiments described above.

This invention provides a method for controlling plant diseases caused by fungal plant pathogens comprising applying to the plant or portion thereof, or to the plant seed, a fungicidally effective amount of a compound selected from Formula 1 (including all geometric and stereoisomers, tautomers, N-oxides, and salts thereof). Of note as embodiments of such methods are methods comprising applying a fungicidally effective amount of a compound corresponding to any of the compound embodiments described above. Of particular note are embodiments where the compounds are applied as compositions of this invention.

One or more of the following methods and variations as described in Schemes 1-21 can be used to prepare the compounds of Formula 1. The definitions of R¹, R², R³, R⁴, R⁵, R⁶, A, A¹, E, E¹, G, Q, W, W¹, X, Y and Z in the compounds of Formulae 1-30 below are as defined above in the Summary of the Invention unless otherwise noted. Compounds of Formulae 1a-1d are various subsets of Formula 1, and all substituents for Formulae 1a-1d are as defined above for Formula 1 unless otherwise noted.

As shown in Scheme 1, compounds of Formula 1a (Formula 1 wherein E is E-1) wherein A is CH(R¹¹) or C(═O) and W is O can be prepared by coupling an acid chloride of Formula 2 with an amine of Formula 3 in the presence of an acid scavenger. Typical acid scavengers include amine bases such as triethylamine, N,N-diisopropylethylamine and pyridine. Other scavengers include hydroxides such as sodium and potassium hydroxide and carbonates such as sodium and potassium carbonate. In some cases the addition of a polymer-supported acid scavenger such as polymer-bound N,N-diisopropylethylamine and polymer-bound 4-dimethylaminopyridine promotes reactivity. Acid salts of the Formula 3 amines can also be used in this reaction, provided that at least 2 equivalents of the acid scavenger is present. Typical acids used to form salts with amines include hydrochloric acid, oxalic acid and trifluoroacetic acid. Acid chlorides of Formula 2 can be prepared from the corresponding acids using a wide variety of well-known conditions published in the chemistry literature.

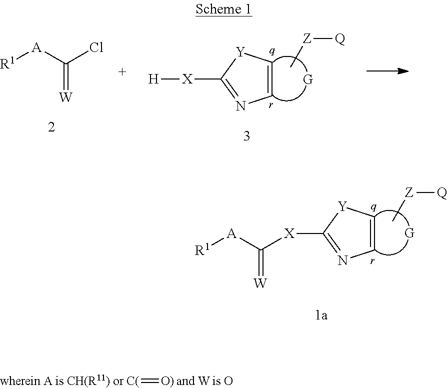

As shown in Scheme 2, compounds of Formula 1a (Formula 1 wherein E is E-1) wherein A is CH(R¹¹) or C(═O) and W is O can also be prepared by coupling an amine of Formula 3 (or its acid salt) with an acid of Formula 4 in the presence of a dehydrative coupling reagent such as N,N-dicyclohexylcarbodiimide (DCC), 1-(3-dimethylaminopropyl)-3-ethylcarbodiimide hydrochloride (EDC) or O-benzotriazol-1-yl-N,N,N′,N′-tetramethyl-uronium hexafluorophosphate (HBTU). Polymer-supported reagents are also useful, such as polymer-bound cyclohexylcarbodiimide derivatives. The method of Scheme 2 is typically conducted in a suitable solvent such as dichloromethane or acetonitrile and in the presence of a base such as triethylamine or N,N-diisopropylethylamine at a temperature between about 0 and 40° C. For conditions and variations of this reaction see, for example, PCT Patent Publication WO 2009/094445 Example 6 (Step D), Example 7 and Example 8.

Acids of Formula 4 are commercially available and can be prepared by methods known in the art. For example, R¹ CH₂COOH where R¹ is linked to the acetic acid residue through a heteroatom can be prepared by reacting the corresponding compound of formula R¹H with a haloacetic acid or ester in the presence of base; see, for example, U.S. Pat. No. 4,084,955. R¹CH₂COOH wherein R¹ is linked to the acetic acid residue through a carbon atom can be prepared from the corresponding compound of formula R¹CH₂-halogen by displacement of the halogen with cyanide followed by hydrolysis; see, for example, Adachi, Yuki Gosei Kagaku Kyokaishi 1969, 27(9), 875-876; or from R¹C(═O)CH₃ using Willgerodt-Kindler reaction conditions; see, for example, Darabi et al., Tetrahedron Letters 1999, 40(42), 7549-7552 and Alam et al., Synthetic Communications 2003, 33(1), 59-63 and references cited therein; or from R¹Br or R¹I by palladium-catalyzed cross-coupling with tert-butyl acetate or diethyl malonate followed by ester hydrolysis; see, for example Buchwald, et al., J. Am. Chem. Soc. 2001, 123(33), 7996-8002 and Hartwig et al., J. Am. Chem. Soc. 2002, 124(42), 12557-12565.

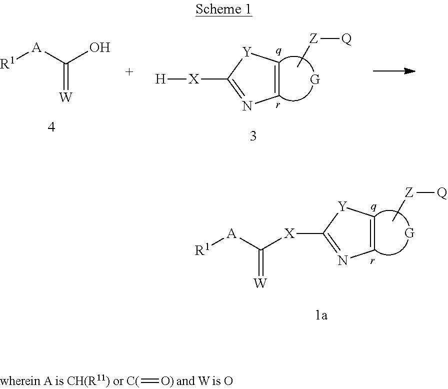

One skilled in the art will recognize that the methods of Schemes 1 and 2 can result in mixtures when certain other functionalities are present in the compound of Formula 3 (e.g., when a second NH group is present). In these instances, incorporation of a protection/deprotection sequence or standard separation methods can be employed to isolate the desired product.

As shown in Scheme 3, compounds of Formula 1a (Formula 1 wherein E is E-1) wherein A is CH(R¹¹) or CO═O, W is O and R¹ is linked to A through a heteroatom can be prepared by reacting a compound of Formula 5 with a compound of Formula 6 wherein L¹ is Cl, Br or I. The reaction is carried out in the presence of a base such as sodium hydride, potassium carbonate or triethylamine and a solvent such as tetrahydrofuran, N,N-dimethylformamide or acetonitrile at a temperature between about 0 to 80° C.

Compounds of Formula 5 are known and can be prepared by methods known in the art; see, for example, Dayagi et al., in The Chemistry of the Carbon-Nitrogen Double Bond, ed. Patei, Interscience, New York 1970; Sandler et al., Organic Functional Group Preparations, Academic Press, New York 1972, 3, 372 and Hilgetag et al., Preparative Organic Chemistry, John Wiley & Sons, New York 1972, 504-515. Compounds of Formula 6 wherein A is C(R¹¹) can be prepared by reacting an amine of Formula 3 with an α-halocarboxylic acid halide or an α-halocarboxylic acid (or its anhydride), using conditions analogous to those described for the amide-forming reactions in Schemes 1 and 2. Compounds of Formula 6 wherein A is C(═O) can be prepared by reacting an amine of Formula 3 and oxalyl chloride by methods well-known in the art.

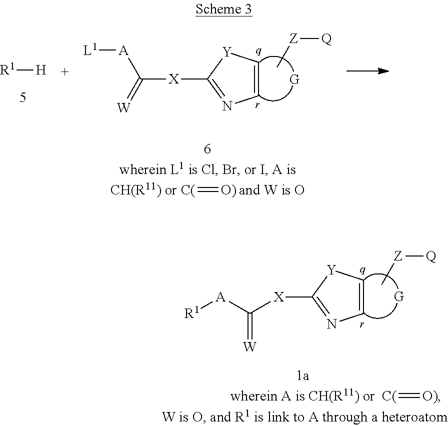

As depicted in Scheme 4, compounds of Formula 1a (Formula 1 wherein E is E-1) wherein A is NH can be prepared by reacting an amine of Formula 3 with an isocyanate of formula R¹NCO or isothiocyanate of formula R¹NCS to obtain compounds of Formula 1a wherein W is O or S, respectively. This reaction is typically carried out at an ambient temperature in an aprotic solvent such as dichloromethane or acetonitrile. For conditions and variations of this reaction see, for example, PCT Patent Publication WO 2009/094445 Example 1 (Step C), Example 4 and Example 5.

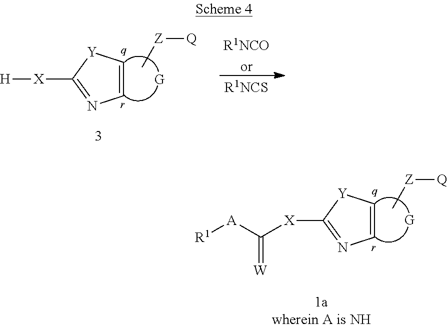

Compounds of Formula 1a (Formula 1 wherein E is E-1) wherein A is NH can also be prepared by reacting an amine of Formula 7 with a compound of Formula 8 (wherein L² is Cl or imidazol-1-yl) as illustrated in Scheme 5. When L² is Cl, the reaction is typical carried out in the presence of an acid scavenger such as an amine base (e.g., triethylamine, N,N-diisopropylethylamine and pyridine). Other scavengers include hydroxides such as sodium and potassium hydroxide and carbonates such as sodium and potassium carbonate. Compounds of Formula 8 wherein L² is Cl can be prepared from amines of Formula 3 by treatment with phosgene (for W═O) or thiophosgene (for W═S), or their equivalents. Compounds of Formula 8 wherein L² is imidazol-1-yl can be prepared from amines of Formula 3 by treatment with 1,1′-carbonyldiimidazole (for W═O) or 1,1′-thiocarbonyldiimidazole (for W═S), according to general methods known to one skilled in the art.

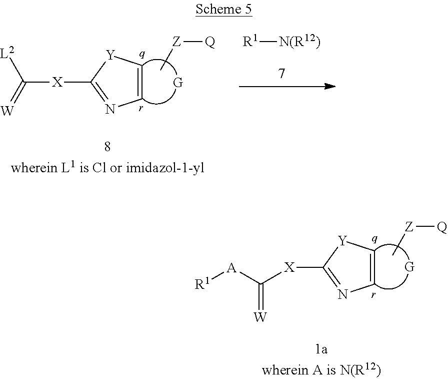

As shown in Scheme 6, compounds of Formula 1b (Formula 1 wherein E is E-2) wherein W is O can be prepared by coupling an amine of Formula 3 with an acid chloride of Formula 9 in the presence of an acid scavenger, analogous to the method described in Scheme 1. Acid chlorides of Formula 9 can be prepared from the corresponding acids using a wide variety of well-known conditions published in the chemistry literature.

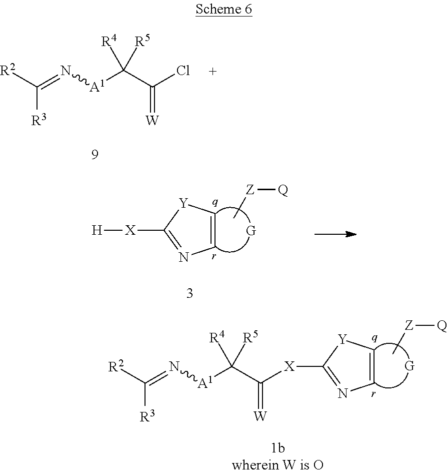

In an alternate method, as depicted in Scheme 7, compounds of Formula 1b (Formula 1 wherein E is E-2) wherein W is O can be prepared by coupling an amine of Formula 3 (or its acid salt) with an acid of Formula 10 in the presence of a dehydrative coupling reagent analogous to the method described in Scheme 2. Acids of Formula 10 are known and can be prepared by methods known to one skilled in the art. For leading references see, for example, Schumann, Paquette et al., Journal of Medicinal & Pharmaceutical Chemistry 1962, 5, 464-77; Van Dijk et al., Journal of Medicinal Chemistry 1977, 20(9), 1199-206; Balsamo et al., Journal of Medicinal Chemistry 1989, 32(6), 1398-1401; and U.S. Pat. No. 4,584,014.

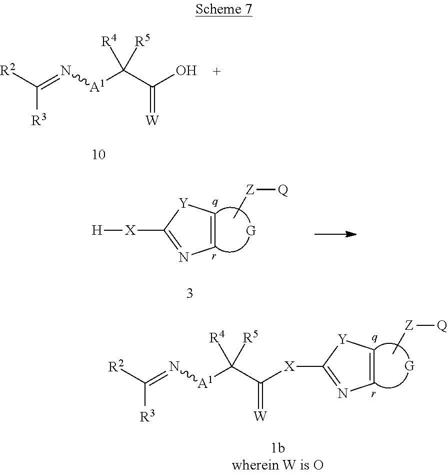

Compounds of Formula 1b (Formula 1 wherein E is E-2) wherein A¹ is O, S or N(R¹³) and W is O can be prepared by reacting a compound of Formula 11 and a haloacetamide of Formula 12 (wherein L¹ is Cl, Br or I) as shown in Scheme 8. The reaction is carried out in the presence of a base such as sodium hydride or potassium carbonate and a solvent such as tetrahydrofuran, N,N-dimethylformamide or acetonitrile typically at a temperature between about 0 to 80° C.

Compounds of Formula 11 are known and can be prepared by methods known in the art; see, for example, Dayagi et al., in The Chemistry of the Carbon-Nitrogen Double Bond, ed. Patei, Interscience, New York 1970; Sandler et al., Organic Functional Group Preparations, Academic Press, New York 1972, 3, 372 and Hilgetag et al., Preparative Organic Chemistry, John Wiley & Sons, New York 1972, 504-515. Haloacetamide compounds of Formula 12 can be prepared by reacting an amine of Formula 3 with an α-halocarboxylic acid halide or an α-halocarboxylic acid or its anhydride, analogous to the amide-forming reactions described in Schemes 1 and 2, respectively.

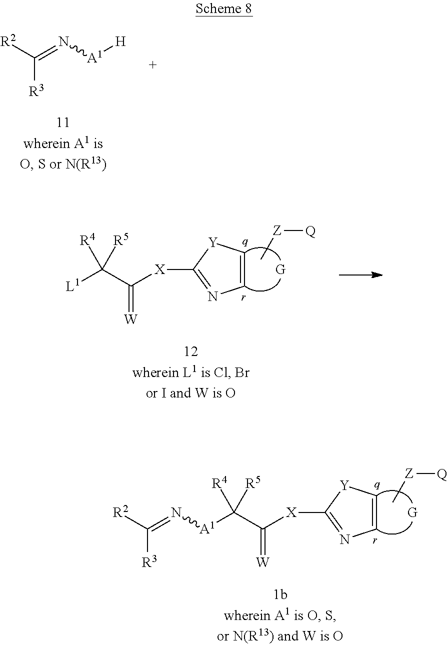

Compounds of Formula 1b (Formula 1 wherein E is E-2) wherein A¹ is —OC(R¹⁴)₂—, —SC(R¹⁴)₂— or —N(R¹³)C(R¹⁴)²— and R⁵ is H can be prepared by a base-catalyzed condensation reaction of a compound of Formula 11 with an α,β-unsaturated amide of Formula 12 as depicted in Scheme 9. In this method A¹ in Formula 11 and C(R¹⁴)₂ in Formula 12 form A¹ in Formula 1b. The reaction is carried out in the presence of a base such as sodium or potassium hydroxide, sodium hydride or potassium carbonate in a solvent such as tetrahydrofuran, N,N-dimethylformamide, ethanol or acetonitrile typically at a temperature between about 0 to 80° C. The α,β-unsaturated amides of Formula 12 can be prepared by coupling the corresponding α,β-unsaturated acids or acid chlorides with amines of Formula 3 using conditions analogous to those described for Schemes 1 and 2.

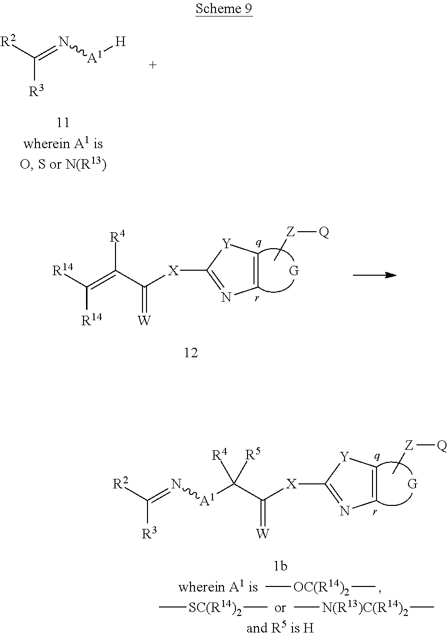

Compounds of Formula 1b (Formula 1 wherein E is E-2) wherein A¹ is —OC(R¹⁴)₂—, —SC(R¹⁴)₂— or —N(R¹³)C(R¹⁴)₂— can also be prepared by reacting a compound of Formula 13 with a compound of Formula 14 as illustrated in Scheme 10. The reaction is carried out in a solvent such as ethanol, tetrahydrofuran or water, and optionally in the presence of an acid catalyst such as acetic acid, hydrochloric acid or sulfuric acid. Acid salts of Formula 14 compounds can also be used in this method, preferably in the presence of at least one molar equivalent of an acid scavenger such as pyridine or triethylamine. Typical acids used to form salts with amines include hydrochloric acid, oxalic acid and trifluoroacetic acid. The reaction of amines with carbonyl compounds is well-known see, for example, Dayagi et al. in The Chemistry of the Carbon-Nitrogen Double Bond, ed. Patei, Interscience, New York 1970; Sandler et al., Organic Functional Group Preparations, Academic Press, New York 1972, 3, 372 and Hilgetag et al., Preparative Organic Chemistry, John Wiley & Sons, New York 1972, 504-515. Compounds of Formula 13 are known and can be prepared by methods known to one skilled in the art. Compounds of Formula 14 can be prepared directly or by deprotection of corresponding N-protected compounds of Formula 14. The N-protected compounds of Formula 14 can be prepared by methods analogous to those already described for Schemes 1-4. The choice and use of a suitable N-protected nitrogen function will be apparent to one skilled in the art; methods for protecting nitrogen atoms with these protecting groups are described in Greene, T. W.; Wuts, P. G. M. Protective Groups in Organic Synthesis, 2nd ed.; Wiley: New York, 1991.

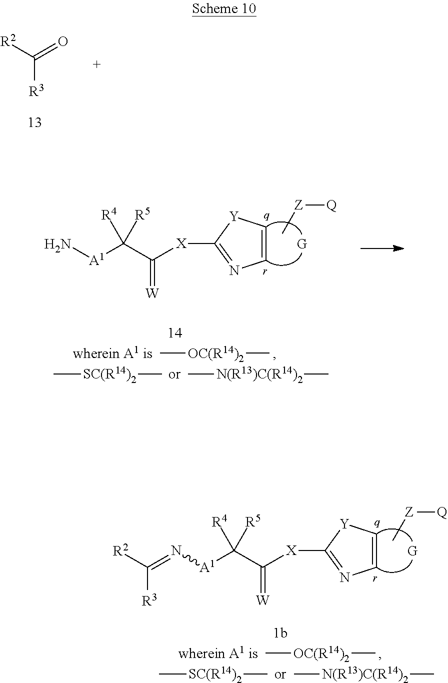

As shown in Scheme 11, compounds of Formula 1c (Formula 1 wherein E is E-3) wherein W¹ is OR¹⁵, SR¹⁶, NR¹⁷R¹⁸ or CN can be prepared by reacting an imidoyl chloride of Formula 15 with a compound of Formula 16 in the presence of an acid scavenger. Suitable acid scavengers include, but are not limited to, amine bases such as triethylamine, N,N-diisopropylethylamine and pyridine, hydroxides such as sodium and potassium hydroxide, and carbonates such as sodium and potassium carbonate. Alternatively, the compounds of Formulae 15 and 16 can be contacted in the absence of an acid scavenger to provide compounds Formula 1c as the corresponding HCl salts, which are also compounds of the present invention. If desired, the HCl salts can be free-based by standard methods to give compounds of Formula 1c. Regardless of whether the reaction is conducted with or without an acid scavenger, it is typically conducted in a suitable organic solvent at a temperature between about −20 and 100° C. A variety of solvents can be used to form the suitable solvent for this method, for example nitriles, such as acetonitrile, ethers such as tetrahydrofuran, and halogenated hydrocarbons such as dichloromethane, and amides such as N,N-dimethylformamide, and mixtures thereof. Compounds of Formula 1c wherein W¹ is OR¹⁵, SR¹⁶, NR¹⁷R¹⁸ or CN can be generally classified as isoureas, isothioureas, guanidines and cyanoamidines, respectively. For leading references on these classes of compounds see Mathias, Organic Preparations and Procedures International 1980, 12(5), 309-326; Comprehensive Organic Chemistry, vol. 2, I. O. Sutherland, Ed., Pergamon Press, Oxford; Rodd's Chemistry of Carbon Compounds, vol. 1C, Elsevier, New York; Katritzky et al., J. Organic Chem. 2004, 69, 309-313. One skilled in the art will recognize that compounds of Formula 1c wherein W¹ is OR¹⁵, NR¹⁷R¹⁸ or CN can be prepared from the corresponding compounds of Formula 1c wherein W¹ is SR¹⁶ by treatment with an appropriate compound of Formula 16. The preparation of thiuronium salts and their conversion to guanidines is described in the literature; see, for example, see Rasmussen et al., Synthesis 1988, 6, 460-466. For conditions and variations of this reaction see, for example, PCT Patent Publication WO 2009/094445 Example 3 and Example 9 (Step C).

Imidoyl chlorides of Formula 15 can be prepared by treating compounds of Formula 1a (Formula 1 wherein E is E-1) wherein A is NH with thionyl chloride, phosphorous oxychloride or phosphorous pentachloride in a solvent such as dichloromethane. For typical reactions conditions see, for example, Zielinski et al., Heterocycles 1998, 48, 319-327. Many compounds of Formula 16 are commercially available and can be prepared by methods well documented in the chemistry art.

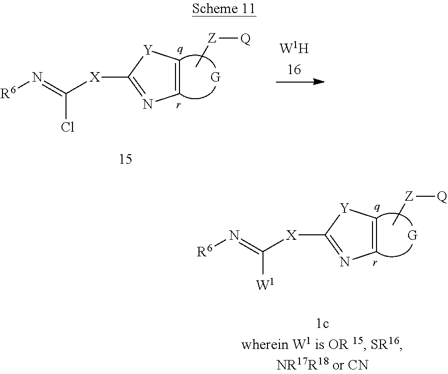

As shown in Scheme 12, compounds of Formula 1c (Formula 1 wherein E is E-3) can also be prepared by reacting an amine of Formula 3 with an imidoyl chloride of Formula 17 using conditions analogous to those described in Scheme 11. Imidoyl chlorides of Formula 17 can be prepared by methods disclosed in the art; see, for example, Bonnett in The Chemistry of the Carbon-Nitrogen Double Bond, Patei, Ed., Interscience Publishers, and references cited therein. Some imidoyl chlorides of Formula 17 are commercially available (e.g., Formula 17 wherein R⁶ is phenyl, substituted phenyl or lower alkyl and W¹ is MeO, MeS, or N(Me)₂ can be commercial obtained) and can be prepared by methods documented in the chemistry art.

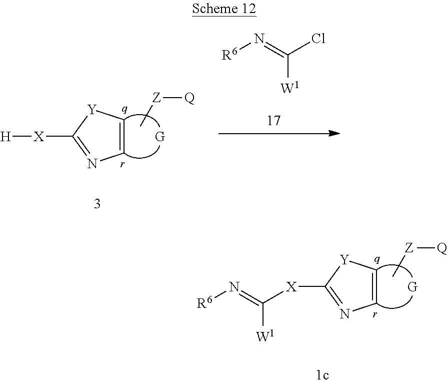

In another method, as shown in Scheme 13, compounds of Formula 1c (Formula 1 wherein E is E-3) wherein W¹ is SR¹⁶ can also be prepared by reacting a thiourea of Formula 1a (Formula 1 wherein E is E-1) wherein A is NH and W is S with an alkylating or acylating agent of a Formula 18 wherein L³ is a nucleophilic reaction leaving group such as halide (e.g., Cl, Br, I) or sulfonate (e.g., mesylate, triflate, p-toluenesulfonate), and the like. The method is conducted in the presence of an acid scavenger and a suitable organic solvent at a temperature between about 0 and 100° C. Suitable solvents include, for example, dichloromethane, tetrahydrofuran, acetonitrile, N,N-dimethylformamide, and mixtures thereof. Suitable acid scavengers comprise, for example, amine bases such as triethylamine, N,N-diisopropylethylamine and pyridine, hydroxides such as sodium and potassium hydroxide and carbonates such as sodium and potassium carbonate. Alternatively, compounds of Formulae 1a and 18 can be contacted in the absence of an acid scavenger to provide the corresponding isothiuronium salts of Formula 1c, which are also compounds of the present invention. In a subsequent reaction the salt can be free-based using standard methods described in the art to provide compounds of Formula 1c. For an example illustrating the preparation of thiuronium salts and their conversion to guanidines see Rasmussen et al., Synthesis 1988, 6, 460-466, and PCT Patent Publication WO 2009/094445 Example 1 (Step D). Many compounds of Formula 18 are known and can be prepared by general methods disclosed in the art.

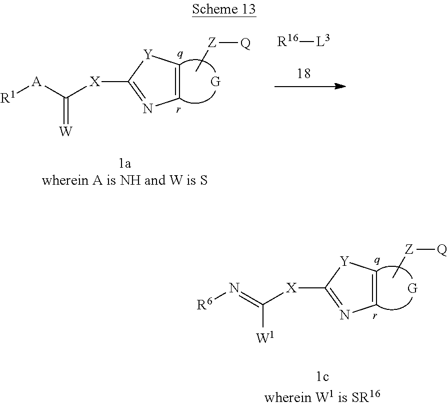

In another method, compounds of Formula 1c (Formula 1 wherein E is E-3) where W¹ is SR¹⁶ can be prepare by reacting an amine of Formula 3 with a dithiocarbamic acid of Formula 19 as illustrated in Scheme 14. The reaction is typically conducted in a suitable solvent at a temperature between about 0 to 100° C. Examples of suitable solvents include acetonitrile, tetrahydrofuran, dichloromethane, N,N-dimethylformamide, and mixtures thereof. Dithiocarbamic acids of Formula 19 can be prepared from the corresponding amines, carbon disulfide and two equivalents of a base, followed by treatment with an alkylating agent according to the general method of Alvarez-Ibarra et al., Organic Preparations and Procedures 1991, 23(5), 611-616.

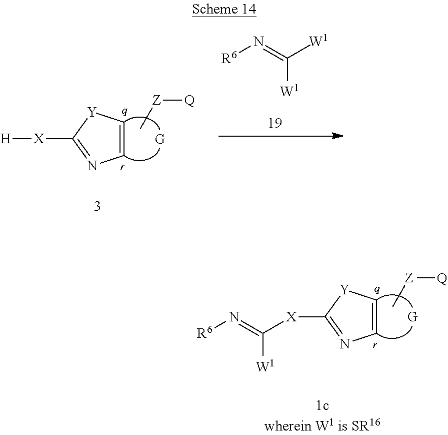

Compounds of Formula 1c (Formula 1 wherein E is E-3) wherein W¹ is H can be prepared by treating an amine of Formula 3 with an imine of Formula 20 as shown in Scheme 15. Imines of Formula 20 can be obtained from the corresponding amines. The procedure involves heating the amines with trimethyl orthoformate or triethyl orthoformate in toluene or xylenes in the presence of a catalytic amount of p-toluenesulfonate.

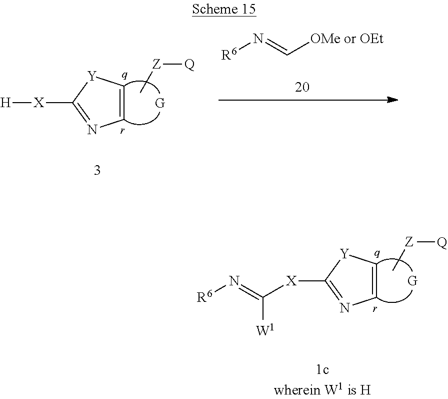

Compounds of Formula 1 wherein X is X², X¹⁰ or X¹¹ can be prepared by reacting a compound of Formula 22 with a of Formula 21 (wherein L⁴ is halide or triflate) as shown in Scheme 16. The reaction is carried out in the presence of a base such as potassium carbonate and in a solvent such as dimethylsulfoxide, N,N-dimethylformamide or acetonitrile at a temperature between about 0 to 80° C. Compounds of Formula 21 can be prepared from corresponding compounds of Formula 21 wherein L⁴ is OH or NH₂ by methods known to one skilled in the art.

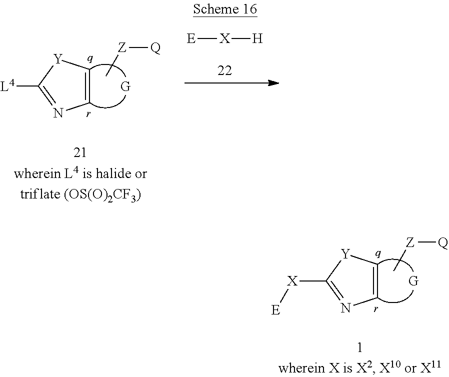

As shown in Scheme 17, compounds of Formula 1 can be prepared reacting a compound of Formula 23 with a compound of Formula 24 wherein Z^{a} and Z^{b} are suitable functional groups which under the appropriate reaction conditions will allow the construction of the various Z groups. Suitable functional groups include, but are not limited to, ionizable carbon-bound hydrogen (e.g., a hydrogen atom connected to a carbon atom adjacent to a C(═O) moiety), carbonyl, aldehyde, ketone, ester, acid, acid chloride, amine, alcohol, thiol, hydrazine, oxime, olefin, acetylene, halide, alkyl halide, methanesulfonate, trifluoromethanesulfonate, boronic acid, boronate, and the like. For example, compounds of Formula 1 wherein Z is CH₂ can be prepared by reacting a compound of Formula 23 wherein Z^{a} is hydrogen (i.e. an ionizable carbon-bound hydrogen adjacent to a C(═O) ring member of the G ring) with a strong base such as lithium diisopropylamide (LDA) or sodium hydride (NaH), followed by a compound of Formula 24 wherein Z^{b} is an methyl halide (e.g., BrCH₂—); while treatment with a compound of Formula 24 wherein Z^{b} is CH(═O)— will give a compound of Formula 1 wherein Z is —CH(OH)—, which can be dehydrated to give a compound of Formula 1 wherein Z is ═CH—. Compounds of Formula 1 wherein Z is O can be prepared by reacting a compound of Formula 23 wherein Z^{a} is Br with a compound of Formula 24 wherein Z^{b} is OH in the presence of a base such as NaH. Compounds of Formula 1 wherein Z is ═NNH— can be prepared by reacting a compound of Formula 23 wherein Z^{a} is a carbonyl (i.e. C(═O) ring member of G) with a compound of Formula 24 wherein Z^{a} is NH₂NH—. Compounds of Formula 1 wherein Z is —CH₂O— can be prepared by reacting a compound of Formula 23 wherein Z^{a} is BrCH₂— with a compound of Formula 24 wherein Z^{b} is OH in the presence of a base. Compounds of Formula 1 wherein Z is —OCH₂CH₂— can be prepared by reacting a compound of Formula 23 wherein Z^{a} is OH with a compound of Formula 24 wherein Z^{b} is ethyl halide (e.g., ICH₂CH₂—) in the presence of a base. The synthetic literature describes many general methods for forming a saturated, partially unsaturated or fully unsaturated chain containing 1- to 3-atoms consisting of carbon and heteroatoms such as the Z groups of the present invention; see, for example, Comprehensive Organic Functional Group Transformations, Vol. 1, 2, 3 and 5, A. R. Katritzky editor, Pergamon Press, New York, 1995; Vogel's Textbook of Practical Organic Chemistry, 5^{th} Ed., pp 470-823, Longman Group, London, 1989; and Advanced Organic Chemistry, 4^{th} Ed. Jerry March, Wiley, New York 1992. Also, Example 2 (Step C) and Example 3 illustrate the method of Scheme 17. One skilled in the art can easily determine how to select an appropriate compound of Formula 23 and Formula 24 to construction a desired Z group. Compounds of Formula 24 are known or can be prepared by methods known in the art.

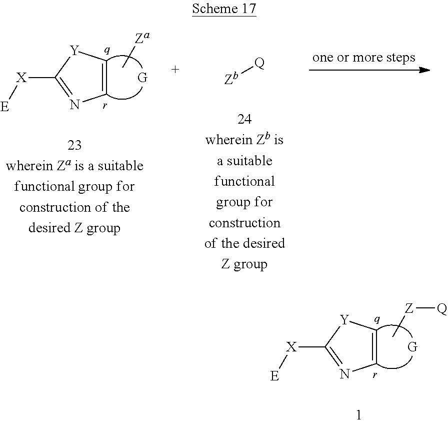

As shown in Scheme 18, compounds of Formula 1 can also be prepared by reacting a compound of Formula 25 with a compound of Formula 26 wherein Ya, Yb and YC are suitable functional groups which under the appropriate reaction conditions will allow the construction of the fused 5-membered heterocyclic ring containing Y. Suitable functional groups include, but are not limited to, hydroxy, thiol, amine, carbonyl, aldehyde, ester, acid, acid chloride, amide, thioamide, cyano, halide, alkyl halide, and the like. The synthetic literature describes many general methods for forming fused 5-membered heterocyclic rings; see, for example, Heterocyclic Compounds, Vol. 5, R. C. Elderfield, Ed., Wiley, New York. 1957, which describes methods to prepare benzofused oxazoles, thiazoles and imidazoles; Comprehensive Heterocyclic Chemistry, Vol. 4-6, A. R. Katritzky and C. W. Rees editors, Pergamon Press, New York, 1984; Comprehensive Heterocyclic Chemistry II, Vol. 2-4, A. R. Katritzky, C. W. Rees, and E. F. Scriven editors, Pergamon Press, New York, 1996; and the series, The Chemistry of Heterocyclic Compounds, E. C. Taylor, editor, Wiley, New York. Also, PCT Patent Publication WO 2010/114971 provides examples for preparing fused 5-membered heterocyclic rings relevant to the present invention. Also, Step B of Example 1 illustrates the method of Scheme 18. One skilled in the art can easily determine how to select an appropriate compound of Formula 25 and Formula 26 to construct the desired fused 5-membered heterocyclic ring.

One skilled in the art will recognize that the method of Scheme 18 can also be performed when the substituent —Z-Q in Formula 26 is replaced with Z^{a} thus providing a compound of Formula 23, which can be reacted with a compound of Formula 24 as described in Scheme 17. Example 2, Step B illustrates this method for preparing a compound of Formula 23.

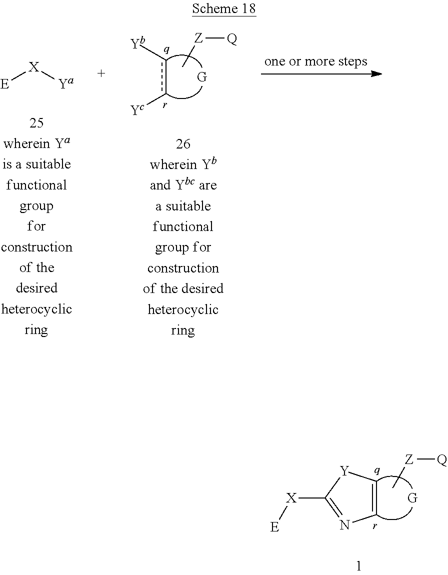

Scheme 19 illustrates a specific example of the general method of Scheme 18 for the preparation of a compound of Formula 1d (Formula 1 wherein E is E¹, X is X¹, Y is S, Z is CH, Q is optionally substituted phenyl and G is G-15 as shown in Exhibit 1). In this method a thioamide of Formula 27 is reacted with a hydroxy bromide of 28 in a solvent such as N,N-dimethylformamide at a temperature between about 20 to 100° C. for about 2 to 24 hours. Compounds of Formula 27 can be prepared by using general procedures disclosed in PCT Patent Publications WO 2008/013925, WO 2008/091580 and WO 2010/065579. Compound 28 can be prepared by bromination of the corresponding keto-lactam.

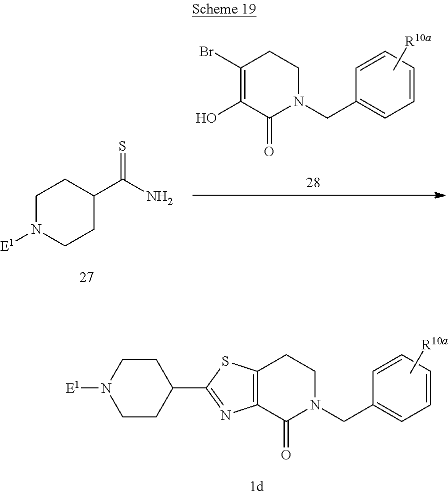

Scheme 20 illustrates a specific example of the general method of Scheme 18 when the substituent —Z-Q in Formula 26 is replaced with Z^{a}. In this example a compound of Formula 23a (Formula 23 wherein E is E¹, X is X¹, Y is S and G is G-15 as shown in Exhibit 1) is prepared by reacting a thioamide of Formula 27 with a compound of Formula 29 in a solvent such as acetone at a temperature between about 20 to 55° C. for about 2 to 24 hours. Compounds of Formula 29 can be prepared by bromination of the corresponding diketone.

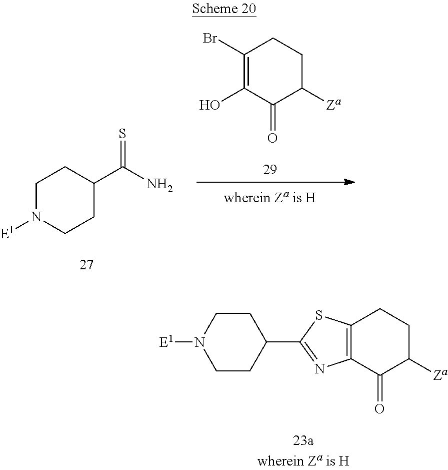

As shown in Scheme 21, the methods of Schemes 17 through 19 can also be performed when the substituent E or E¹ is replaced with an amine-protecting group, which can be removed to provide amines of Formula 3. A wide variety of amine-protecting groups are useful, as the only requirement is for the group to be displaceable to give Formula 3. For examples of appropriate protecting groups see T. W. Greene and P. G. M. Wuts, Protective Groups in Organic Synthesis, 2nd ed., John Wiley & Sons, Inc., New York, 1991. The protecting group can be removed and the amine isolated as either an acid salt or free-amine by general methods known in the art; see, for example PCT Patent Publication WO 2009/09445 Example 1 (Step B) and Example 6 (Step C).

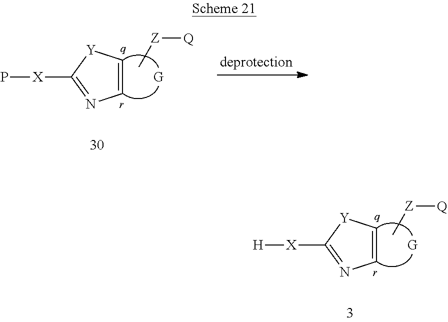

wherein P is an amine protecting group

Numerous other methods for preparation of compounds of Formula 1 and useful intermediates for their preparation exist in the art and are well-known to one skilled in the art. For representative procedures relevant to constructing rings X¹ through X¹¹; see, for example, Comprehensive Heterocyclic Chemistry, Vol. 3 and 7, A. R. Katritzky and C. W. Rees editors, Pergamon Press, New York, 1984; Comprehensive Heterocyclic Chemistry II, Vol. 6 and 9, A. R. Katritzky, C. W. Rees, and E. F. Scriven editors, Pergamon Press, New York, 1996; and the series, The Chemistry of Heterocyclic Compounds, E. C. Taylor, editor, Wiley, New York. For specific examples see methods outlined PCT Patent Publication WO 2011/085170.

It is recognized by one skilled in the art that various functional groups can be converted into others to provide different compounds of Formula 1. For example, conversion of compounds of Formula 1 wherein W is O to the corresponding compounds wherein W is S can be accomplished using a variety of standard thiating reagents such as phosphorus pentasulfide or 2,4-bis(4-methoxyphenyl)-1,3-dithia-2,4-diphosphetane-2,4-disulfide (Lawesson's reagent).

It is recognized that some reagents and reaction conditions described above for preparing compounds of Formula 1 may not be compatible with certain functionalities present in the intermediates. In these instances, the incorporation of protection/deprotection sequences or functional group interconversions into the synthesis will aid in obtaining the desired products. The use and choice of the protecting groups will be apparent to one skilled in chemical synthesis (see, for example, Greene, T. W.; Wuts, P. G. M. Protective Groups in Organic Synthesis, 2nd ed.; Wiley: New York, 1991). One skilled in the art will recognize that, in some cases, after the introduction of a given reagent as it is depicted in any individual scheme, it may be necessary to perform additional routine synthetic steps not described in detail to complete the synthesis of compounds of Formula. One skilled in the art will also recognize that it may be necessary to perform a combination of the steps illustrated in the above schemes in an order other than that implied by the particular sequence presented to prepare the compounds of Formula 1.

One skilled in the art will also recognize that compounds of Formula 1 and the intermediates described herein can be subjected to various electrophilic, nucleophilic, radical, organometallic, oxidation, and reduction reactions to add substituents or modify existing substituents.

Without further elaboration, it is believed that one skilled in the art using the preceding description can utilize the present invention to its fullest extent. The following Synthesis Examples are, therefore, to be construed as merely illustrative, and not limiting of the disclosure in any way whatsoever. Steps in the following Synthesis Examples illustrate a procedure for each step in an overall synthetic transformation, and the starting material for each step may not have necessarily been prepared by a particular preparative run whose procedure is described in other Examples or Steps. Percentages are by weight except for chromatographic solvent mixtures or where otherwise indicated. Parts and percentages for chromatographic solvent mixtures are by volume unless otherwise indicated. ¹H NMR spectra are reported in ppm downfield from tetramethylsilane in CDCl₃ unless otherwise noted; “s” means singlet, “t” means triplet, “m” means multiplet, “dd” means doublet of doublets.

### Example 1

### Preparation of 6,7-dihydro-2-[1-[2-[5-methyl-3-(trifluoromethyl)-1H-pyrazol-1-yl]acetyl]-4-piperidinyl]-5-(phenylmethyl)thiazolo[4,5-c]pyridin-4(5H)-one (Compound No. 8)

### Step A: Preparation of 4-bromo-5,6-dihydro-3-hydroxy-1-(phenylmethyl)-2(1H)-pyridinone

A mixture of 1-(phenylmethyl)-2,3-piperidinedione (1.15 g, 5.0 mmol) in diethyl ether (15 mL) and tetrahydrofuran (15 mL) was cooled in an ice-water bath, and then bromine (0.80 g, 5.0 mmol) was added dropwise. The reaction mixture was stirred for 1 h, and then warmed to room temperature and stirred for an additional 2 h. The reaction mixture was concentrated under reduced pressure and the resulting material was purified by medium pressure liquid chromatography on silica gel (0 to 100% gradient of ethyl acetate in hexanes as eluant) to give a solid (1.6 g). The solid was dissolved in hot diethyl ether and allowed to cool to provide the title compound as a white solid (0.68 g).

¹H NMR (CDCl₃): δ 2.77 (t, 2H), 3.37 (t, 2H), 4.61 (s, 2H), 6.81 (s, 1H), 7.20-7.40 (m, 5H).

### Step B: Preparation of 6,7-dihydro-2-[1-[2-[5-methyl-3-(trifluoromethyl)-1H-pyrazol-1-yl]acetyl]-4-piperidinyl]-5-(phenylmethyl)thiazolo[4,5-c]pyridin-4(5H)-one

A mixture of 4-bromo-5,6-dihydro-3-hydroxy-1-(phenylmethyl)-2(1H)-pyridinone (i.e. the product of Step A) (0.28 g, 1.0 mmol) and 1-[2-[5-methyl-3-(trifluoromethyl)-1H-pyrazol-1-yl]acetyl]-4-piperidinecarbothioamide (prepared by the method described in PCT Patent Publication WO 20078/091580) (0.33 g, 1.0 mmol) in N,N-dimethylformamide (1.0 mL) was placed on an orbital shaker for 3 days, after which time the reaction mixture was added portionwise to ice water. The resulting solid precipitate was collected on a sintered glass frit funnel. The solid was dissolved in dichloromethane, dried over magnesium sulfate, filtered and concentrated under reduced pressure to a tan solid (0.45 g). The tan solid was purified by medium pressure liquid chromatography on silica gel (0 to 100% gradient of ethyl acetate in hexanes, then 20% methanol in ethyl acetate as eluant) to provide a green oil (0.28 g). The green oil was dissolved in ethyl acetate and filtered through a pad of silica gel (2.0 g). The filtrate was concentrated under reduced pressure to provide the title, a compound of the present invention, compound as a foamy-tan solid (0.18 g).

¹H NMR (CDCl₃): 6.65-1.85 (m, 2H), 2.10-2.25 (m, 2H), 2.32 (s, 3H), 2.83 (m, 1H), 3.04 (m, 2H), 3.20-3.40 (m, 2H), 3.57 (m, 2H), 4.03 (m, 1H), 4.58 (m, 1H), 4.76 (s, 2H), 4.99 (dd, 2H), 6.32 (s, 1H), 7.25-7.40 (m, 5H).

### Example 2

### Preparation of 5-[(2,6-difluorophenyl)methyl]-6,7-dihydro-2-[1-[2-[5-methyl-3-(trifluoromethyl)-1H-pyrazol-1-yl]acetyl]-4-piperidinyl]-4(5H)-benzonthiazolone (Compound No. 5)

### Step A: Preparation of 3-bromo-2-hydroxy-2-cyclohenxen-1-one

A mixture of 1,2-cyclohexanedione (1.12 g, 10.0 mmol) in diethyl ether (50 mL) was cooled in an ice-water bath, and then bromine (1.60 g, 10.0 mmol) was added dropwise. The reaction mixture was stirred for 10 minutes with ice-water bath cooling, and then concentrated under reduced pressure. The resulting material was purified by medium pressure liquid chromatography on silica gel (0 to 100% gradient of ethyl acetate in hexanes as eluant) to provide the title compound as a white solid (1.3 g).

¹H NMR (CDCl₃): δ 2.05-2.15 (m, 2H), 2.50-2.60 (m, 2H), 2.83-2.92 (m, 2H), 6.40 (s, 1H).

### Step B: Preparation of 6,7-dihydro-2-[1-[2-[5-methyl-3-(trifluoromethyl)-1H-pyrazol-1-yl]acetyl]-4-piperidinyl]-4(5H)-benzothiazole

A mixture of 3-bromo-2-hydroxy-2-cyclohenxen-1-one (i.e. the product of Step A) (1.30 g, 6.8 mmol) and 1-[2-[5-methyl-3-(trifluoromethyl)-1H-pyrazol-1-yl]acetyl]-4-piperidinecarbothioamide (prepared by the method described in PCT Patent Publication WO 20078/091580) (2.27 g, 6.8 mmol) in acetone (30 mL) was stirred overnight, and then heated at reflux for 24 h. The reaction mixture was cooled to room temperature and sodium bicarbonate (1.0 g) was added. After 1 h, the reaction mixture was filtered and concentrated under reduced pressure. The resulting material was partitioned between water and ethyl acetate and the layers were separated. The organic layer was dried over magnesium sulfate, filtered and concentrated under reduced pressure to give a foamy-white solid (3.13 g). The solid was purified by medium pressure liquid chromatography on silica gel (0 to 100% gradient of ethyl acetate in hexanes as eluant) to provide the title compound as a solid (0.54 g).

¹H NMR (CDCl₃): δ 1.65-1.85 (m, 2H), 2.10-2.30 (m, 4H), 2.30 (s, 3H), 2.65 (m, 2H), 2.82 (m, 1H), 3.10 (m, 2H), 3.20-3.35 (m, 2H), 4.02 (m, 1H), 4.58 (m, 1H), 5.00 (m, 2H), 6.33 (s, 1H).

### Step C: Preparation of 5-[(2,6-difluorophenyl)methyl]-6,7-dihydro-2-[1-[2-[5-methyl-3-(trifluoromethyl)-1H-pyrazol-1-yl]acetyl]-4-piperidinyl]-4(5H)-benzonthiazolone

To a mixture of 6,7-dihyrdo-2-[1-[2-[5-methyl-3-(trifluoromethyl)-1H-pyrazol-1-yl]acetyl]-4-piperidinyl]-4(5H)-benzothiazolone (i.e. the product of Step B) (0.46 g, 1.08 mmol) in tetrahydrofuran (2 mL) cooled to −70° C. was added dropwise lithium diisopropylamide (1.6 M in hexanes, 170 μL, 1.20 mmol, freshly prepared). When the addition was complete, more tetrahydrofuran (2 mL) was added to the reaction mixture. The reaction mixture was stirred at −70° C. for 30 minutes, and then a solution of 2-(bromomethyl)-1,3-difluorobenezene (0.22 g, 1.08 mmol) in tetrahydrofuran (1 mL) was added dropwise. The reaction mixture was allowed to gradually warm to room temperature and stirred overnight. The reaction mixture was diluted with aqueous hydrochloric acid solution (1 N, 1 mL) and water, and then extracted with dichloromethane. The organic extract was dried over magnesium sulfate, filtered and concentrated under reduced pressure to give an orange oil (0.70 g). The oil was purified (2×) by medium pressure liquid chromatography on silica gel (0 to 100% gradient of ethyl acetate in hexanes as eluant) to provide the title, a compound of the present invention, compound as a foamy-yellow solid (0.16 g).

¹H NMR (CDCl₃) δ 1.65-1.85 (m, 2H), 2.10-2.30 (m, 4H), 2.33 (s, 3H), 2.70-2.90 (m, 3H), 2.90-3.05 (m, 1H), 3.10-3.20 (m, 1H), 3.20-3.35 (m, 2H), 3.52 (m, 1H), 4.03 (m, 1H), 4.58 (m, 1H), 4.98 (m, 2H), 6.33 (s, 1H), 6.88 (m, 2H), 7.18 (m, 1H).

### Example 3

### Preparation of 5-[(2,6-difluorophenyl)methyl]-6,7-dihydro-2-[1-[2-[5-methyl-3-(trifluoromethylene)-1H-pyrazol-1-yl]acetyl]-4-piperidinyl]-4(5H)-benzothiazolone (Compound No. 6)

A mixture of 6,7-dihyrdo-2-[1-[2-[5-methyl-3-(trifluoromethyl)-1H-pyrazol-1-yl]acetyl]-4-piperidinyl]-4(5H)-benzothiazolone (i.e. the product of Example 2, Step B) (0.71 g, 1.66 mmol), 2,6-difluorobenzaldehyde (0.24 g, 1.70 mmol) and calcium hydroxide (0.018 g, 0.25 mmol) in acetonitrile (50 mL) was heated at reflux for 2 days with the use of an extractor containing activated 3 Å molecular sieves. The reaction mixture was cooled, concentrated under reduced pressure and the resulting material purified by medium pressure liquid chromatography on silica gel (0 to 100% gradient of ethyl acetate in hexanes as eluant) to provide the title, a compound of the present invention, compound as a yellow oil (0.28 g).

¹H NMR (CDCl₃) δ 1.65-1.85 (m, 2H), 2.10-2.30 (m, 2H), 2.33 (s, 3H), 2.80-2.98 (m, 3H), 3.12 (m, 2H), 3.22-3.40 (m, 2H), 4.03 (m, 1H), 4.61 (m, 1H), 4.98 (m, 2H), 6.34 (s, 1H), 6.88 (m, 2H), 7.35 (m, 1H), 7.57 (s, 1H).

By the procedures described herein, together with methods known in the art, the following compounds of Tables 1 to 12-H can be prepared. The following abbreviations are used in the Tables: n means normal, i means iso, c means cyclo, Me means methyl, MeO means methoxy, MeS means methylthio, Et means ethyl, EtO means ethoxy, c-Pr means cyclopropyl, Bu means butyl, c-Bu means cyclobutyl, i-BuO means isobutoxy, CN means cyano, Ph means phenyl and NO₂ means nitro.

TABLE 1

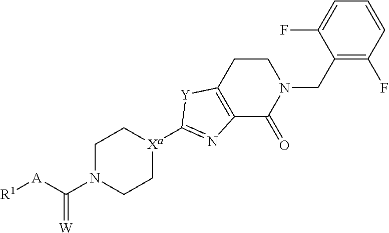

| A is CH₂, W is O, X^{a} is CH and | A is CH₂, W is O, X^{a} is CH and |
|---|---|
| Y is S. | Y is S. |
| R¹ | R¹ |
| Ph | i-BuO |
| 2-Me—Ph | CF₃CH₂OCH₂ |
| 2-MeO—Ph | 3-Et—Ph |
| 2-Cl—Ph | 3-CF₃—Ph |
| 2-Br—Ph | 3-CN—Ph |
| 2-EtO—Ph | 3-NO₂—Ph |
| 2-MeS—Ph | 2,5-di-Cl—Ph |
| 3-Cl—Ph | 5-Br-2-Cl—Ph |
| 3-Br—Ph | 2-Cl-5-Me—Ph |
| 3-I—Ph | 2-MeO-5-CF₃—Ph |
| 3-Me—Ph | 2,5-di-Et—Ph |
| 2-Cl-5-CF₃—Ph | 3-Me-1H-pyrazol-1-yl |
| 2,5-di-Br—Ph | 3-Cl-1H-pyrazol-1-yl |
| 2-Br-5-Me—Ph | 3-Br-1H-pyrazol-1-yl |
| 2-Br-5-CF₃—Ph | 3-CF₃-1H-pyrazol-1-yl |
| 5-Cl-2-Me—Ph | 3,5-di-Me-1H-pyrazol-1-yl |
| 5-Br-2-Me—Ph | 3-Cl-5-Me-1H-pyrazol-1-yl |
| 2,5-di-Me—Ph | 3-Br-5-Me-1H-pyrazol-1-yl |
| 2-Me-5-CF₃—Ph | 5-MeO-3-Me-1H-pyrazol-1-yl |
| 5-CN-2-Me—Ph | 3,5-di-Et-1H-pyrazol-1-yl |
| 2-Me-5-NO₂—Ph | 5-Et-3-CF₃-1H-pyrazol-1-yl |
| 5-Cl-2-MeO—Ph | 2,5-di-Me-3-furyl |
| 5-Br-2-MeO—Ph | 2,5-di-Me-3-thienyl |
| 2-MeO-5-Me—Ph | 2,5-di-Cl-3-thienyl |
| 3-Et-5-Me-1H-pyrazol-1-yl | 1,4-di-Me-1H-pyrrol-3-yl |
| 5-Me-3-CF₃-1H-pyrazol-1-yl | 1,4-di-Me-1H-pyrazol-3-yl |
| 5-Me-3-CF₃CF₂-1H-pyrazol-1-yl | 1,3-di-Me-4-1H-pyrazol-4-yl |
| 5-Cl-3-Me-1H-pyrazol-1-yl | 2,5-di-Me-4-oxazolyl |
| 3,5-di-Cl-1H-pyrazol-1-yl | 2,5-di-Me-4-thiazolyl |
| 5-Cl-3-CF₃-1H-pyrazol-1-yl | 3,6-di-Me-2-pyridinyl |
| 5-Br-3-Me-1H-pyrazol-1-yl | 2,5-di-Me-3-pyridinyl |
| 3,5-di-Br-1H-pyrazol-1-yl | 2,5-di-Me-4-pyridinyl |
| 5-Br-3-CF₃-1H-pyrazol-1-yl | 3,6-di-Cl-2-pyridinyl |
| 3-CHF₂-1H-pyrazol-1-yl | 2,5-di-Cl-3-pyridinyl |
| 3-CHF₂-5-Me-1H-pyrazol-1-yl | 2,5-di-Cl-4-pyridinyl |
| 3,5-bis-(CHF₂)-1H-pyrazol-1-yl | 4-Br-3-pyridazinyl |
| 3,5-di-Me-2-thienyl | 4-CF₃-2-pyrimidinyl |
| 3,5-di-Cl-2-thienyl | 3,6-di-Me-2-pyrazinyl |
| 3,5-di-Me-2-furyl | 2,5-di-Me-4-pyrimidinyl |
| 4-Me-2-CF₃-5-thiazolyl | 4-MeO-5-pyrimidinyl |
| 4-Me-2-CF₃-5-oxazolyl | 3,6-di-Me-4-pyridazinyl |
| 1-Me-4-CF₃-1H-imidazol-2-yl | 1-Me-4-CF₃-1H-imidazol-2-yl |
| 2,4-di-Me-1H-pyrrol-1-yl | 3,5-bis-(CF₃)-1H-pyrazol-1-yl |
| 1-Me-3-CF₃-1H-pyrazol-5-yl | 3-Cl-5-CF₃-1H-pyrazol-1-yl |
| 3-Br-5-CF₃-1H-pyrazol-1-yl | 3,5-bis-(CHF₂O)-1H-pyrazol-1-yl |
| 3-Me-5-CF₃-1H-pyrazol-1-yl | 3,5-di-MeO-1H-pyrazol-1-yl |
| 3-MeO-5-CF₃-1H-pyrazol-1-yl | 5-EtO-3-Me-1H-pyrazol-1-yl |
| 3,5-di-Br-1H-pyrazol-1-yl | 5-EtO-3-CF₃-1H-pyrazol-1-yl |
| 5-MeO-3-Me-1H-pyrazol-1-yl | 3,5-di-Br-1H-1,2,4-triazol-1-yl |
| 5-MeO-3-CF₃-1H-pyrazol-1-yl | 3-Cl-5-Me-1H-1,2,4-triazol-1-yl |
| 3,5-di-Cl-1H-1,2,4-triazol-1-yl | 3-Br-5-Me-1H-1,2,4-triazol-1-yl |
| 3-Me-5-Cl-1H-1,2,4-triazol-1-yl | 3-CF₃-5-Cl-1H-1,2,4-triazol-1-yl |
| 3-Me-5-Br-1H-1,2,4-triazol-1-yl | 3-CF₃-5-Br-1H-1,2,4-triazol-1-yl |
| 3-Cl-5-CF₃-1H-1,2,4-triazol-1-yl | 3,5-bis-(CF₃)-1H-1,2,4-triazol-1-yl |
| 3-Br-5-CF₃-1H-1,2,4-triazol-1-yl | CF₃OCH₂CH₂ |
| n-Bu | MeOCH₂CH₂O |
| (Me)₂CHCH₂CH₂ | CF₃CH₂CH₂O |
| CH₃C(Me)═CHCH₂ | CF₃CH₂C(═O)O |
| HC≡CCH₂ | CH₂═CHCH₂O |
| CF₃CH₂CH₂CH₂ | CH₃CH₂CH₂S |
| Cl₂C═CHCH₂ | CF₃CH₂CH₂S |
| 2-CF₃-c-Pr | CF₃CH₂CH₂NH |

The present disclosure also includes Tables 1-A through 1-Q, each of which are constructed the same as Table 1 above except that the row heading in Table 1 (i.e. “A is CH₂, W is O, X^{a} is CH and Y is S”) is replaced with the respective row headings shown below. For example, in Table 1-A the row heading is “A is NH, W is O, X^{a} is CH and Y is S” and R¹ is as defined in Table 1 above. Thus, the first entry in Table 1-A specifically discloses 4-[5-[(2,6-difluorophenyl)methyl]-4,5,6,7-tetrahydro-4-oxothiazolo[4,5-c]pyridin-2-yl]-N-phenyl-1-piperidinecarboxamide. Tables 1-B through 1-Q are constructed similarly.

| Table | Row Heading |
|---|---|
| 1-A | A is NH, W is O, X^{a} is CH and Y is S. |
| 1-B | A is CH₂, W is O, X^{a} is N and Y is S. |
| 1-C | A is NH, W is O, X^{a} is N and Y is S. |
| 1-D | A is CH₂, W is O, X^{a} is CH and Y is O. |
| 1-E | A is NH, W is O, X^{a} is CH and Y is O. |
| 1-F | A is CH₂, W is O, X^{a} is N and Y is O. |
| 1-G | A is NH, W is O, X^{a} is N and Y is O. |
| 1-H | A is CH₂, W is O, X^{a} is CH and Y is NH. |
| 1-I | A is NH, W is O, X^{a} is CH and Y is NH. |
| 1-J | A is CH₂, W is O, X^{a} is N and Y is NH. |
| 1-K | A is NH, W is O, X^{a} is N and Y is NH. |
| 1-L | A is CH₂, W is O, X^{a} is CH and Y is N(Me). |
| 1-M | A is NH, W is O, X^{a} is CH and Y is N(Me). |
| 1-N | A is CH₂, W is O, X^{a} is N and Y is N(Me). |
| 1-O | A is NH, W is O, X^{a} is N and Y is N(Me). |
| 1-P | A is CH₂, W is S, X^{a} is CH and Y is S. |
| 1-Q | A is NH, W is S, X^{a} is CH and Y is S. |

TABLE 1a

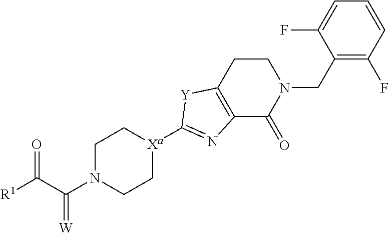

| W is O, X^{a} is CH and Y is S. | W is O, X^{a} is CH and Y is S. |
|---|---|
| R¹ | R¹ |
| Ph | CF₃CH₂CH₂CH₂ |
| 2-Me—Ph | Cl₂C═CHCH₂ |
| 2-MeO—Ph | 2-CF₃-c-Pr |
| 2-Cl—Ph | CF₃CH₂OCH₂ |
| 2-Br—Ph | 3-Et—Ph |
| 2-EtO—Ph | 3-CF₃—Ph |
| 2-MeS—Ph | 3-CN—Ph |
| 3-Cl—Ph | 3-NO₂—Ph |
| 3-Br—Ph | 2,5-di-Cl—Ph |
| 3-I—Ph | 5-Br-2-Cl—Ph |
| 3-Me—Ph | 2-Cl-5-Me—Ph |
| 2-Cl-5-CF₃—Ph | 2-MeO-5-CF₃—Ph |
| 2,5-di-Br—Ph | 2,5-di-Et—Ph |
| 2-Br-5-Me—Ph | 2,5-di-Me-3-furyl |
| 2-Br-5-CF₃—Ph | 2,5-di-Me-3-thienyl |
| 5-Cl-2-Me—Ph | 2,5-di-Cl-3-thienyl |
| 5-Br-2-Me—Ph | 1,4-di-Me-1H-pyrrol-3-yl |
| 2,5-di-Me—Ph | 1,4-di-Me-1H-pyrazol-3-yl |
| 2-Me-5-CF₃—Ph | 1,3-di-Me-4-1H-pyrazol-4-yl |
| 5-CN-2-Me—Ph | 2,5-di-Me-4-oxazolyl |
| 2-Me-5-NO₂—Ph | 2,5-di-Me-4-thiazolyl |
| 5-Cl-2-MeO—Ph | 3,6-di-Me-2-pyridinyl |
| 5-Br-2-MeO—Ph | 2,5-di-Me-3-pyridinyl |
| 2-MeO-5-Me—Ph | 2,5-di-Me-4-pyridinyl |
| 3,5-di-Me-2-thienyl | 3,6-di-Cl-2-pyridinyl |
| 3,5-di-Cl-2-thienyl | 2,5-di-Cl-3-pyridinyl |
| 3,5-di-Me-2-furyl | 2,5-di-Cl-4-pyridinyl |
| 4-Me-2-CF₃-5-thiazolyl | 4-Br-3-pyridazinyl |
| 4-Me-2-CF₃-5-oxazolyl | 4-CF₃-2-pyrimidinyl |
| 1-Me-4-CF₃-1H-imidazol-2-yl | 3,6-di-Me-2-pyrazinyl |
| 1-Me-3-CF₃-1H-pyrazol-5-yl | 2,5-di-Me-4-pyrimidinyl |
| n-Bu | 4-MeO-5-pyrimidinyl |
| (Me)₂CHCH₂CH₂ | 3,6-di-Me-4-pyridazinyl |
| CH₃C(Me)═CHCH₂ | 1-Me-4-CF₃-1H-imidazol-2-yl |
| HC≡CCH₂ | CF₃OCH₂CH₂ |

The present disclosure also includes Tables 1^{a}-A through 1^{a}-G, each of which are constructed the same as Table 1^{a} above except that the row heading in Table 1^{a} (i.e. “W is O, X^{a} is CH and Y is S”) is replaced with the respective row headings shown below. For example, in Table 1^{a}-A the row heading is “W is O, X^{a} is N and Y is S” and R¹ is as defined in Table 1 above. Tables 1^{a}-B through 1^{a}-G are constructed similarly.

| Table | Row Heading |
|---|---|
| 1^{a}-A | W is O, X^{a} is N and Y is S. |
| 1^{a}-B | W is O, X^{a} is CH and Y is O. |

TABLE 2

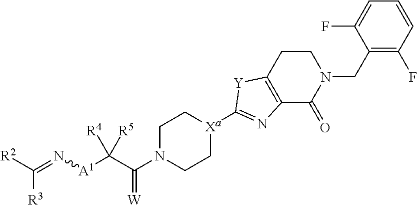

| R² | R³ | A¹ | R⁴ | R⁵ | W |
|---|---|---|---|---|---|
| CH₃ | CH₃ | O | H | H | O |
| CH₃ | CH₃ | S | H | H | O |
| CH₃ | CH₃ | NH | H | H | O |
| CH₃ | CH₃ | N(Me) | H | H | O |
| CH₃ | CH₃ | CH₂ | H | H | O |
| CH₃ | CH₃ | —OCH₂— | H | H | O |
| CH₃ | CH₃ | —SCH₂— | H | H | O |
| CH₃ | CH₃ | —NHCH₂— | H | H | O |
| CH₃ | CH₃ | —N(Me)CH₂— | H | H | O |
| CH₃ | CH₃ | O | CH₃ | H | O |
| CH₃ | CH₃ | O | CH₃ | CH₃ | O |
| CH₃ | CH₃ | O | H | H | S |
| CF₃ | H | O | H | H | O |
| CF₃ | H | S | H | H | O |
| CF₃ | H | NH | H | H | O |
| CF₃ | H | N(Me) | H | H | O |
| CF₃ | H | CH₂ | H | H | O |
| CF₃ | H | —OCH₂— | H | H | O |
| CF₃ | H | —SCH₂— | H | H | O |
| CF₃ | H | —NHCH₂— | H | H | O |
| CF₃ | H | —N(Me)CH₂— | H | H | O |
| CF₃ | CH₃ | O | H | H | O |
| CF₃ | CH₃ | S | H | H | O |
| CF₃ | CH₃ | NH | H | H | O |
| CF₃ | CH₃ | N(Me) | H | H | O |
| CF₃ | CH₃ | CH₂ | H | H | O |
| CF₃ | CH₃ | —OCH₂— | H | H | O |
| CF₃ | CH₃ | —SCH₂— | H | H | O |
| CF₃ | CH₃ | —NHCH₂— | H | H | O |
| CF₃ | CH₃ | —N(Me)CH₂— | H | H | O |
| CF₃ | H | O | CH₃ | H | O |
| CF₃ | CH₃ | O | H | CH₃ | O |
| CF₃CH₂ | H | O | H | H | O |
| CF₃CH₂ | CH₃ | O | H | H | O |
| CH₃CH₂ | H | O | H | H | O |
| CH₃CH₂ | CH₃ | O | H | H | O |
| CH₃ | H | O | H | H | O |
| CHF₂ | H | O | H | H | O |
| CHF₂ | CH₃ | O | H | H | O |
| CHF₂ | CHF₂ | O | H | H | O |

| | | | | |
|---|---|---|---|---|
| CH₃ | —CH₂CH(Me)N— | H | H | O |
| CF₃ | —CH₂CH(Me)N— | H | H | O |

X^{a} is CH and Y is S.

The present disclosure also includes Tables 2-A through 2-G, each of which are constructed the same as Table 2 above except that the row heading in Table 2 (i.e. “X^{a} is CH and Y is S”) is replaced with the respective row headings shown below. For example, in Table 2-A the row heading is “X^{a} is N and Y is S” and R², R³, A¹, R⁴, R⁵ and W are as defined in Table 2 above. Thus, the first entry in Table 2-A specifically discloses 5-[(2,6-difluorophenyl)methyl]-6,7-dihydro-2-[4-[2-[[(1-methylethylidene)amino]oxy]acetyl]-1-piperazinyl]thiazolo[4,5-c]pyridin-4(5H)-one. Tables 2-B through 2-G are constructed similarly.

| Table | Row Heading |
|---|---|
| 2-A | X^{a} is N and Y is S. |
| 2-B | X^{a} is CH and Y is O. |
| 2-C | X^{a} is N and Y is O. |
| 2-D | X^{a} is CH and Y is NH. |
| 2-E | X^{a} is N and Y is NH. |
| 2-F | X^{a} is CH and Y is N(Me). |
| 2-G | X^{a} is N and Y is N(Me). |

TABLE 3

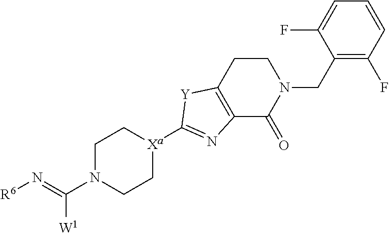

| R⁶ | W¹ | R⁶ | W¹ |
|---|---|---|---|
| 2-Me—Ph | MeO | 2-Me—Ph | NHOH |
| 2-MeO—Ph | MeO | 2-MeO—Ph | NHOH |
| 2-Cl—Ph | MeO | 2-Cl—Ph | NHOH |
| 2-Br—Ph | MeO | 2-Br—Ph | NHOH |
| 2-Et—Ph | MeO | 2-Et—Ph | NHOH |
| 2-EtO—Ph | MeO | 2-EtO—Ph | NHOH |
| 2-MeS—Ph | MeO | 2-MeS—Ph | NHOH |
| 2-CF₃O—Ph | MeO | 2-CF₃O—Ph | NHOH |
| 3-Cl—Ph | MeO | 3-Cl—Ph | NHOH |
| 3-Br—Ph | MeO | 3-Br—Ph | NHOH |
| 3-Me—Ph | MeO | 3-Me—Ph | NHOH |
| 2,5-di-Me—Ph | MeO | 2,5-di-Me—Ph | NHOH |
| 2,5-di-Cl—Ph | MeO | 2,5-di-Cl—Ph | NHOH |
| 2-Cl-5-CF₃—Ph | MeO | 2-Cl-5-CF₃—Ph | NHOH |
| 2,5-di-Br—Ph | MeO | 2,5-di-Br—Ph | NHOH |
| 2-Br-5-CF₃—Ph | MeO | 2-Br-5-CF₃—Ph | NHOH |
| 5-Cl-2-Me—Ph | MeO | 5-Cl-2-Me—Ph | NHOH |
| 5-Br-2-Me—Ph | MeO | 5-Br-2-Me—Ph | NHOH |
| 2-Me-5-CF₃—Ph | MeO | 2-Me-5-CF₃—Ph | NHOH |
| 5-Cl-2-MeO—Ph | MeO | 5-Cl-2-MeO—Ph | NHOH |
| 5-Br-2-MeO—Ph | MeO | 5-Br-2-MeO—Ph | NHOH |
| 2-MeO-5-Me—Ph | MeO | 2-MeO-5-Me—Ph | NHOH |
| 2-MeO-5-CF₃—Ph | MeO | 2-MeO-5-CF₃—Ph | NHOH |
| 2,5-di-Et—Ph | MeO | 2,5-di-Et—Ph | NHOH |
| 3,5-di-Me-1H- | MeO | 3,5-di-Me-1H- | NHOH |
| pyrazol-1-yl | | pyrazol-1-yl | |
| 3,5-di-Cl-1H- | MeO | 3,5-di-Cl-1H- | NHOH |
| pyrazol-1-yl | | pyrazol-1-yl | |
| 3,5-di-Br-1H- | MeO | 3,5-di-Br-1H- | NHOH |
| pyrazol-1-yl | | pyrazol-1-yl | |
| 3,5-bis-(CF₃)-1H- | MeO | 3,5-bis-(CF₃)-1H- | NHOH |
| pyrazol-1-yl | | pyrazol-1-yl | |
| 5-Me-3-(CF₃-1H- | MeO | 5-Me-3-(CF₃-1H- | NHOH |
| pyrazol-1-yl | | pyrazol-1-yl | |
| 3-CHF₂-1H- | MeO | 3-CHF₂-1H- | NHOH |
| pyrazol-1-yl | | pyrazol-1-yl | |
| 3-CHF₂-5-Me-1H- | MeO | 3-CHF₂-5-Me-1H- | NHOH |
| pyrazol-1-yl | | pyrazol-1-yl | |
| 3,5-bis-(CHF₂)-1H- | MeO | 3,5-bis-(CHF₂)-1H- | NHOH |
| pyrazol-1-yl | | pyrazol-1-yl | |
| 3,5-di-Me-1H-1,2,4- | MeO | 3,5-di-Me-1H-1,2,4- | NHOH |
| triazol-1-yl | | triazol-1-yl | |
| 3,5-di-Cl-1H-1,2,4- | MeO | 3,5-di-Cl-1H-1,2,4- | NHOH |
| triazol-1-yl | | triazol-1-yl | |
| 3,5-di-Br-1H-1,2,4- | MeO | 3,5-di-Br-1H-1,2,4- | NHOH |
| triazol-1-yl | | triazol-1-yl | |
| n-Bu | MeO | n-Bu | NHOH |
| (Me)₂CHCH₂CH₂ | MeO | (Me)₂CHCH₂CH₂ | NHOH |
| CH₃C(Me)═CHCH₂ | MeO | CH₃C(Me)═CHCH₂ | NHOH |
| HC≡CCH₂ | MeO | HC≡CCH₂ | NHOH |
| CF₃CH₂CH₂CH₂ | MeO | CF₃CH₂CH₂CH₂ | NHOH |
| Cl₂C═CHCH₂ | MeO | Cl₂C═CHCH₂ | NHOH |
| 2-CF₃-c-Pr | MeO | 2-CF₃-c-Pr | NHOH |
| i-BuO | MeO | i-BuO | NHOH |
| CF₃OCH₂CH₂ | MeO | CF₃OCH₂CH₂ | NHOH |
| CF₃CH₂CH₂O | MeO | CF₃CH₂CH₂O | NHOH |
| 2-Me—Ph | MeS | 2-Me—Ph | MeONH |
| 2-MeO—Ph | MeS | 2-MeO—Ph | MeONH |
| 2-Cl—Ph | MeS | 2-Cl—Ph | MeONH |
| 2-Br—Ph | MeS | 2-Br—Ph | MeONH |
| 2-Et—Ph | MeS | 2-Et—Ph | MeONH |
| 2-EtO—Ph | MeS | 2-EtO—Ph | MeONH |
| 2-MeS—Ph | MeS | 2-MeS—Ph | MeONH |
| 2-CF₃O—Ph | MeS | 2-CF₃O—Ph | MeONH |
| 3-Cl—Ph | MeS | 3-Cl—Ph | MeONH |
| 3-Br—Ph | MeS | 3-Br—Ph | MeONH |
| 3-Me—Ph | MeS | 3-Me—Ph | MeONH |
| 2,5-di-Me—Ph | MeS | 2,5-di-Me—Ph | MeONH |
| 2,5-di-Cl—Ph | MeS | 2,5-di-Cl—Ph | MeONH |
| 2-Cl-5-CF₃—Ph | MeS | 2-Cl-5-CF₃—Ph | MeONH |
| 2,5-di-Br—Ph | MeS | 2,5-di-Br—Ph | MeONH |
| 2-Br-5-CF₃—Ph | MeS | 2-Br-5-CF₃—Ph | MeONH |
| 5-Cl-2-Me—Ph | MeS | 5-Cl-2-Me—Ph | MeONH |
| 5-Br-2-Me—Ph | MeS | 5-Br-2-Me—Ph | MeONH |
| 2-Me-5-CF₃—Ph | MeS | 2-Me-5-CF₃—Ph | MeONH |
| 5-Cl-2-MeO—Ph | MeS | 5-Cl-2-MeO—Ph | MeONH |
| 5-Br-2-MeO—Ph | MeS | 5-Br-2-MeO—Ph | MeONH |
| 2-MeO-5-Me—Ph | MeS | 2-MeO-5-Me—Ph | MeONH |
| 2-MeO-5-CF₃—Ph | MeS | 2-MeO-5-CF₃—Ph | MeONH |
| 2,5-di-Et—Ph | MeS | 2,5-di-Et—Ph | MeONH |
| 3,5-di-Me-1H- | MeS | 3,5-di-Me-1H- | MeONH |
| pyrazol-1-yl | | pyrazol-1-yl | |
| 3,5-di-Cl-1H- | MeS | 3,5-di-Cl-1H- | MeONH |
| pyrazol-1-yl | | pyrazol-1-yl | |
| 3,5-di-Br-1H- | MeS | 3,5-di-Br-1H- | MeONH |
| pyrazol-1-yl | | pyrazol-1-yl | |
| 3,5-bis-(CF₃)-1H- | MeS | 3,5-bis-(CF₃)-1H- | MeONH |
| pyrazol-1-yl | | pyrazol-1-yl | |
| 5-Me-3-(CF₃-1H- | MeS | 5-Me-3-(CF₃-1H- | MeONH |
| pyrazol-1-yl | | pyrazol-1-yl | |
| 3-CHF₂-1H- | MeS | 3-CHF₂-1H- | MeONH |
| pyrazol-1-yl | | pyrazol-1-yl | |
| 3-CHF₂-5-Me-1H- | MeS | 3-CHF₂-5-Me-1H- | MeONH |
| pyrazol-1-yl | | pyrazol-1-yl | |
| 3,5-bis-(CHF₂)-1H- | MeS | 3,5-bis-(CHF₂)-1H- | MeONH |
| pyrazol-1-yl | | pyrazol-1-yl | |
| 3,5-di-Me-1H-1,2,4- | MeS | 3,5-di-Me-1H-1,2,4- | MeONH |
| triazol-1-yl | | triazol-1-yl | |
| 3,5-di-Cl-1H-1,2,4- | MeS | 3,5-di-Cl-1H-1,2,4- | MeONH |
| triazol-1-yl | | triazol-1-yl | |
| 3,5-di-Br-1H-1,2,4- | MeS | 3,5-di-Br-1H-1,2,4- | MeONH |
| triazol-1-yl | | triazol-1-yl | |
| n-Bu | MeS | n-Bu | MeONH |
| (Me)₂CHCH₂CH₂ | MeS | (Me)₂CHCH₂CH₂ | MeONH |
| CH₃C(Me)═CHCH₂ | MeS | CH₃C(Me)═CHCH₂ | MeONH |
| HC≡CCH₂ | MeS | HC≡CCH₂ | MeONH |
| CF₃CH₂CH₂CH₂ | MeS | CF₃CH₂CH₂CH₂ | MeONH |
| Cl₂C═CHCH₂ | MeS | Cl₂C═CHCH₂ | MeONH |
| 2-CF₃-c-Pr | MeS | 2-CF₃-c-Pr | MeONH |
| i-BuO | MeS | i-BuO | MeONH |
| CF₃OCH₂CH₂ | MeS | CF₃OCH₂CH₂ | MeONH |
| CF₃CH₂CH₂O | MeS | CF₃CH₂CH₂O | MeONH |
| 2-Me—Ph | NH₂ | 2-Me—Ph | NH₂NH |
| 2-MeO—Ph | NH₂ | 2-MeO—Ph | NH₂NH |
| 2-Cl—Ph | NH₂ | 2-Cl—Ph | NH₂NH |
| 2-Br—Ph | NH₂ | 2-Br—Ph | NH₂NH |
| 2-Et—Ph | NH₂ | 2-Et—Ph | NH₂NH |
| 2-EtO—Ph | NH₂ | 2-EtO—Ph | NH₂NH |
| 2-MeS—Ph | NH₂ | 2-MeS—Ph | NH₂NH |
| 2-CF₃O—Ph | NH₂ | 2-CF₃O—Ph | NH₂NH |
| 3-Cl—Ph | NH₂ | 3-Cl—Ph | NH₂NH |
| 3-Br—Ph | NH₂ | 3-Br—Ph | NH₂NH |
| 3-Me—Ph | NH₂ | 3-Me—Ph | NH₂NH |
| 2,5-di-Me—Ph | NH₂ | 2,5-di-Me—Ph | NH₂NH |
| 2,5-di-Cl—Ph | NH₂ | 2,5-di-Cl—Ph | NH₂NH |
| 2-Cl-5-CF₃—Ph | NH₂ | 2-Cl-5-CF₃—Ph | NH₂NH |
| 2,5-di-Br—Ph | NH₂ | 2,5-di-Br—Ph | NH₂NH |
| 2-Br-5-CF₃—Ph | NH₂ | 2-Br-5-CF₃—Ph | NH₂NH |
| 5-Cl-2-Me—Ph | NH₂ | 5-Cl-2-Me—Ph | NH₂NH |
| 5-Br-2-Me—Ph | NH₂ | 5-Br-2-Me—Ph | NH₂NH |
| 2-Me-5-CF₃—Ph | NH₂ | 2-Me-5-CF₃—Ph | NH₂NH |
| 5-Cl-2-MeO—Ph | NH₂ | 5-Cl-2-MeO—Ph | NH₂NH |
| 5-Br-2-MeO—Ph | NH₂ | 5-Br-2-MeO—Ph | NH₂NH |
| 2-MeO-5-Me—Ph | NH₂ | 2-MeO-5-Me—Ph | NH₂NH |
| 2-MeO-5-CF₃—Ph | NH₂ | 2-MeO-5-CF₃—Ph | NH₂NH |
| 2,5-di-Et—Ph | NH₂ | 2,5-di-Et—Ph | NH₂NH |
| 3,5-di-Me-1H- | NH₂ | 3,5-di-Me-1H- | NH₂NH |
| pyrazol-1-yl | | pyrazol-1-yl | |
| 3,5-di-Cl-1H- | NH₂ | 3,5-di-Cl-1H- | NH₂NH |
| pyrazol-1-yl | | pyrazol-1-yl | |
| 3,5-di-Br-1H- | NH₂ | 3,5-di-Br-1H- | NH₂NH |
| pyrazol-1-yl | | pyrazol-1-yl | |
| 3,5-bis-(CF₃)-1H- | NH₂ | 3,5-bis-(CF₃)-1H- | NH₂NH |
| pyrazol-1-yl | | pyrazol-1-yl | |
| 5-Me-3-(CF₃-1H- | NH₂ | 5-Me-3-(CF₃-1H- | NH₂NH |
| pyrazol-1-yl | | pyrazol-1-yl | |
| 3-CHF₂-1H- | NH₂ | 3-CHF₂-1H- | NH₂NH |
| pyrazol-1-yl | | pyrazol-1-yl | |
| 3-CHF₂-5-Me-1H- | NH₂ | 3-CHF₂-5-Me-1H- | NH₂NH |
| pyrazol-1-yl | | pyrazol-1-yl | |
| 3,5-bis-(CHF₂)-1H- | NH₂ | 3,5-bis-(CHF₂)-1H- | NH₂NH |
| pyrazol-1-yl | | pyrazol-1-yl | |
| 3,5-di-Me-1H-1,2,4- | NH₂ | 3,5-di-Me-1H-1,2,4- | NH₂NH |
| triazol-1-yl | | triazol-1-yl | |
| 3,5-di-Cl-1H-1,2,4- | NH₂ | 3,5-di-Cl-1H-1,2,4- | NH₂NH |
| triazol-1-yl | | triazol-1-yl | |
| 3,5-di-Br-1H-1,2,4- | NH₂ | 3,5-di-Br-1H-1,2,4- | NH₂NH |
| triazol-1-yl | | triazol-1-yl | |
| n-Bu | NH₂ | n-Bu | NH₂NH |
| (Me)₂CHCH₂CH₂ | NH₂ | (Me)₂CHCH₂CH₂ | NH₂NH |
| CH₃C(Me)═CHCH₂ | NH₂ | CH₃C(Me)═CHCH₂ | NH₂NH |
| HC≡CCH₂ | NH₂ | HC≡CCH₂ | NH₂NH |
| CF₃CH₂CH₂CH₂ | NH₂ | CF₃CH₂CH₂CH₂ | NH₂NH |
| Cl₂C═CHCH₂ | NH₂ | Cl₂C═CHCH₂ | NH₂NH |
| 2-CF₃-c-Pr | NH₂ | 2-CF₃-c-Pr | NH₂NH |
| i-BuO | NH₂ | i-BuO | NH₂NH |
| CF₃OCH₂CH₂ | NH₂ | CF₃OCH₂CH₂ | NH₂NH |
| CF₃CH₂CH₂O | NH₂ | CF₃CH₂CH₂O | NH₂NH |

X^{a} is CH and Y is S.

The present disclosure also includes Tables 3-A through 3-G, each of which are constructed the same as Table 3 above except that the Row Heading in Table 3 (i.e. “X^{a} is CH and Y is S”) is replaced with the respective row headings shown below. For example, in Table 3-A the row heading is “X^{a} is N and Y is S” and R⁶ and W¹ are as defined in Table 3 above. Thus, the first entry in Table 3-A specifically discloses methyl 4-[5-[(2,6-difluorophenyl)methyl]-4,5,6,7-tetrahydro-4-oxothiazolo[4,5-c]pyridin-2-yl]-N-(2-methylphenyl)-1-piperazinecarboximidate. Tables 3-B through 3-G are constructed similarly.

| Table | Row Heading |
|---|---|
| 3-A | X^{a} is N and Y is S. |
| 3-B | X^{a} is CH and Y is O. |
| 3-C | X^{a} is N and Y is O. |
| 3-D | X^{a} is CH and Y is NH. |
| 3-E | X^{a} is N and Y is NH. |
| 3-F | X^{a} is CH and Y is N(Me). |
| 3-G | X^{a} is N and Y is N(Me). |

TABLE 4

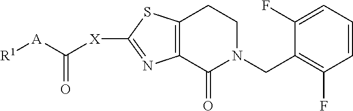

In Table 4 the substituents R^{7a} and R^{7b} are
attached to the X-ring, as defined in the Summary of the
Invention. A dash (“—”) in the R^{7a} and/or R^{7b}
column below indicates that the X-ring is unsubstituted.

| X | R^{7a} | R^{7b} |
|---|---|---|
| X¹ | — | — |
| X² | — | — |
| X³ | — | — |
| X⁴ | — | — |
| X⁵ | — | — |
| X⁶ | — | — |
| X⁷ | — | — |
| X⁸ | — | — |
| X⁹ | — | — |
| X¹⁰ | — | — |
| X¹¹ | — | — |
| X² | 2-Me | — |
| X⁵ | — | Me |
| X⁵ | — | CH₃C(═O)O |
| X² | 3-Me | — |

R¹ is 3-CF₃-5-Me-1H-pyrazol-1-yl and A is CH₂.

The present disclosure also includes Tables 4-A through 4-P, each of which are constructed the same as Table 4 above except that the Row Heading in Table 4 (i.e. “R¹ is 3-CF₃-5-Me-1H-pyrazol-1-yl and A is CH₂”) is replaced with the respective row headings shown below. For example, in Table 4-A the row heading is “R¹ is 3-CF₃-5-Cl-1H-pyrazol-1-yl and A is CH₂” and X, R^{7a} and R^{7b} are as defined in Table 4 above. Thus, the first entry in Table 4-A specifically discloses 5-[(2,6-difluorophenyl)methyl]-6,7-dihydro-2-[1-[2-[5-chloro-3-(trifluoromethyl)-1H-pyrazol-1-yl]acetyl]-4-piperidinyl]thiazolo[4,5-c]pyridin-4(5H)-one. Tables 4-B through 4-P are constructed similarly.

| Table | Row Heading |
|---|---|
| 4-A | R¹ is 3-CF₃-5-Cl-1H-pyrazol-1-yl and A is CH₂. |
| 4-B | R¹ is 3-CF₃-5-Me-1H-pyrazol-1-yl and A is NH. |
| 4-C | R¹ is 3-CHF₂-1H-pyrazol-1-yl and A is CH₂. |
| 4-D | R¹ is 3-CHF₂-5-Me-1H-pyrazol-1-yl and A is CH₂. |
| 4-E | R¹ is 3,5-bis-(CHF₂)-1H-pyrazol-1-yl and A is CH₂. |
| 4-F | R¹ is 3-CF₃-5-Me-1H-1,2,4-triazol-1-yl and A is CH₂. |
| 4-G | R¹ is 3,5-di-Cl-1H-1,2,4-triazol-1-yl and A is CH₂. |
| 4-H | R¹ is 3,5-di-Br-1H-1,2,4-triazol-1-yl and A is CH₂. |
| 4-I | R¹ is 2,5-di-Me—Ph and A is CH₂. |
| 4-J | R¹ is 2,5-di-Me—Ph and A is NH. |
| 4-K | R¹ is 2,5-di-Me—Ph and A is CH(OH). |
| 4-L | R¹ is 2,5-di-Me—Ph and A is C(═O). |
| 4-M | R¹ is CF₃CH₂CH₂O and A is CH₂. |
| 4-N | R¹ is CF₃CH₂OCH₂ and A is CH₂. |
| 4-O | R¹ is CF₃OCH₂CH₂ and A is CH₂. |
| 4-P | R¹ is CF₃CH₂CH₂CH₂ and A is CH₂. |

TABLE 5

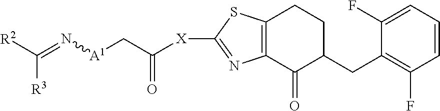

In Table 5 the substituents R^{7a} and R^{7b} are
attached to the X-ring, as defined in the Summary of the
Invention. A dash (“—”) in the R^{7a} and/or R^{7b}
column below indicates that the X-ring is unsubstituted.

| X | R^{7a} | R^{7b} |
|---|---|---|
| X¹ | — | — |
| X² | — | — |
| X³ | — | — |
| X⁴ | — | — |
| X⁵ | — | — |
| X⁶ | — | — |
| X⁷ | — | — |
| X⁸ | — | — |
| X⁹ | — | — |
| X¹⁰ | — | — |
| X¹¹ | — | — |
| X² | 2-Me | — |
| X⁵ | — | Me |
| X⁵ | — | CH₃C(═O)O |
| X² | 3-Me | — |

R² is CF₃, R³ is H and A¹ is O.

The present disclosure also includes Tables 5-A through 5-D, each of which is constructed the same as Table 5 above except that the Row Heading in Table 5 (i.e. “R² is CF₃, R³ is H and A¹ is O”) is replaced with the respective row headings shown below. For example, in Table 5-A the row heading is “R² is CF₃, R³ is Me and A¹ is O” and X, R^{7a} and R^{7b} are as defined in Table 5 above. Thus, the first entry in Table 5-A specifically discloses 5-[(2,6-difluorophenyl)methyl]-6,7-dihydro-2-[1-[2-[[(2,2,2-trifluoro-1-methylethylidene)amino]oxy]acetyl]-4-piperidinyl]-4(5H)-benzothiazolone. Tables 5-B and 5-D are constructed similarly.

| Table | Row Heading |
|---|---|
| 5-A | R² is CF₃, R³ is Me and A¹ is O. |
| 5-B | R² is CF₃, R³ is H and A¹ is N(Me). |
| 5-C | R² is CF₃, R³ is Me and A¹ is N(Me). |
| 5-D | R² is CHF₂, R³ is Me and A¹ is O. |

TABLE 6

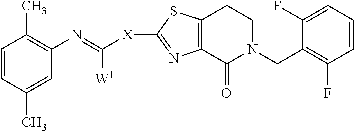

In Table 5 the substituents R^{7a} and R^{7b} are
attached to the X-ring, as defined in the Summary of the
Invention. A dash (“—”) in the R^{7a} and/or R^{7b}
column below indicates that the X-ring is unsubstituted.

| W¹ | X | R^{7a} | R^{7b} | A | X | R^{7a} | R^{7b} |
|---|---|---|---|---|---|---|---|
| MeO | X¹ | — | — | NH₂ | X¹ | — | — |
| MeO | X² | — | — | NH₂ | X² | — | — |
| MeO | X³ | — | — | NH₂ | X³ | — | — |
| MeO | X⁴ | — | — | NH₂ | X⁴ | — | — |
| MeO | X⁵ | — | — | NH₂ | X⁵ | — | — |
| MeO | X⁶ | — | — | NH₂ | X⁶ | — | — |
| MeO | X⁷ | — | — | NH₂ | X⁷ | — | — |
| MeO | X⁸ | — | — | NH₂ | X⁸ | — | — |
| MeO | X⁹ | — | — | NH₂ | X⁹ | — | — |
| MeO | X¹⁰ | — | — | NH₂ | X¹⁰ | — | — |
| MeO | X¹¹ | — | — | NH₂ | X¹¹ | — | — |
| MeO | X² | 2-Me | — | NH₂ | X² | 2-Me | — |
| MeO | X⁵ | — | Me | NH₂ | X⁵ | — | Me |
| MeO | X⁵ | — | CH₃C(═O)O | NH₂ | X⁵ | — | CH₃C(═O)O |
| MeO | X² | 3-Me | — | NH₂ | X² | 3-Me | — |
| MeS | X¹ | — | — | NHOH | X¹ | — | — |
| MeS | X² | — | — | NHOH | X² | — | — |
| MeS | X³ | — | — | NHOH | X³ | — | — |
| MeS | X⁴ | — | — | NHOH | X⁴ | — | — |
| MeS | X⁵ | — | — | NHOH | X⁵ | — | — |
| MeS | X⁶ | — | — | NHOH | X⁶ | — | — |
| MeS | X⁷ | — | — | NHOH | X⁷ | — | — |
| MeS | X⁸ | — | — | NHOH | X⁸ | — | — |
| MeS | X⁹ | — | — | NHOH | X⁹ | — | — |
| MeS | X¹⁰ | — | — | NHOH | X¹⁰ | — | — |
| MeS | X¹¹ | — | — | NHOH | X¹¹ | — | — |
| MeS | X² | 2-Me | — | NHOH | X² | 2-Me | — |
| MeS | X⁵ | — | Me | NHOH | X⁵ | — | Me |
| MeS | X⁵ | — | CH₃C(═O)O | NHOH | X⁵ | — | CH₃C(═O)O |
| MeS | X² | 3-Me | — | NHOH | X² | 3-Me | — |

TABLE 7

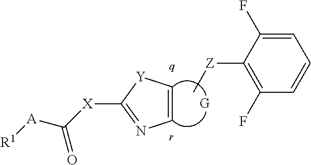

In Table 7 the structures of G-1 through G-30 are
shown in Exhibit 1 above. The substituent R⁸ and is
attached to the G-ring, as defined in the Summary of
the Invention. A dash (“—”) in the R⁸ column below
indicates the G-ring is unsubstituted. The point of
attachment of the G-ring to Z is shown in Exhibit 1 above.

| G | R⁸ | Z | G | R⁸ | Z | G | R⁸ | Z |
|---|---|---|---|---|---|---|---|---|
| G-1 | — | CH₂ | G-27 | — | CH₂ | G-23 | — | OCH₂CH₂ |
| G-2 | — | CH | G-28 | — | CH₂ | G-24 | — | N(Me) |
| G-3 | — | CH₂ | G-29 | — | CH₂ | G-25 | — | CH₂O |
| G-4 | — | CH₂ | G-30 | — | CH₂ | G-26 | — | NHCH(Me) |
| G-5 | — | CH | G-1 | — | CH₂CH₂ | G-27 | — | OCH₂ |
| G-6 | — | CH₂ | G-2 | — | NNH | G-28 | — | SCH₂ |
| G-7 | — | CH₂ | G-3 | — | CH₂CH₂CH₂ | G-29 | — | O |
| G-8 | — | CH | G-4 | — | CH₂CH₂ | G-30 | — | NH |
| G-9 | — | CH₂ | G-5 | — | CHCH₂ | G-13 | 6-Me | CH₂ |
| G-10 | — | CH₂ | G-6 | — | CH₂CH₂ | G-10 | 4-OH | CH₂ |
| G-11 | — | CH | G-7 | — | CH(OH) | G-10 | 4-Me, | CH₂ |
| G-12 | — | CH₂ | G-8 | — | CH(Me) | | 4-OH | |
| G-13 | — | CH₂ | G-9 | — | CH(Me) | G-13 | — | CH₂OCH₂ |
| G-14 | — | CH | G-10 | — | O | G-13 | — | CH₂SCH₂ |
| G-15 | — | CH₂ | G-11 | — | NO | G-13 | — | CH₂NHCH₂ |
| G-16 | — | CH₂ | G-12 | — | CH(C≡N) | G-13 | — | CH₂N(Me)CH₂ |
| G-17 | — | CH | G-13 | — | CH₂O | G-13 | — | CH₂CH₂O |
| G-18 | — | CH₂ | G-14 | — | CHCH₂ | G-13 | — | CH₂CH₂S |
| G-19 | — | CH₂ | G-15 | — | CH(Me) | G-13 | — | CH₂CH₂N(Me) |
| G-20 | — | CH₂ | G-15 | — | CH(Et) | G-19 | — | SCH₂ |
| G-21 | — | CH₂ | G-13 | — | CH₂S | G-19 | — | NHCH₂ |
| G-22 | — | CH₂ | G-13 | — | CH₂CH₂NH | G-19 | — | OCH₂CH₂ |
| G-23 | — | CH₂ | G-19 | — | OCH₂ | G-19 | — | SCH₂CH₂ |
| G-24 | — | CH₂ | G-20 | — | CH₂O | G-19 | — | CH₂O |
| G-25 | — | CH₂ | G-21 | — | NH | G-19 | — | CH₂S |
| G-26 | — | CH₂ | G-22 | — | S | G-19 | — | CH₂NH |
| | | | | | | G-19 | — | CH₂OCH₂ |
| | | | | | | G-19 | — | CH(OH) |

R¹ is 3-CF₃-CF₃-5-Me-1H-pyrazol-1-yl, A is CH₂, X is X¹ and Y is S.

The present disclosure also includes Tables 7-A through 7-X, each of which is constructed the same as Table 7 above except that the Row Heading in Table 7 (i.e. “R¹ is 3-CF₃-5-Me-1H-pyrazol-1-yl, A is CH₂, X is X¹ and Y is S”) is replaced with the respective row headings shown below. For example, in Table 7-A the row heading is “R¹ is 2,5-di-Me-Ph, A is CH₂, X is X¹ and Y is S” and G, R⁸ and Z are as defined in Table 7 above. Thus, the first entry in Table 7-A specifically discloses 1-[4-[5-[(2,6-difluorophenyl)methyl]-5,6-dihydro-4H-cyclopentathiazol-2-yl]-1-piperidinyl]-2-(2,5-dimethylphenyl)ethanone. Tables 7-B and 7-X are constructed similarly.

| Table | Row Heading |
|---|---|
| 7-A | R¹ is 2,5-di-Me-Ph, A is CH₂, X is X¹ and Y is S. |
| 7-B | R¹ is 2,5-di-Me-Ph, A is NH, X is X¹ and Y is S. |
| 7-C | R¹ is 3-CF₃-5-Me-1H-pyrazol-1-yl, A is CH₂, X is X² and Y is S. |
| 7-D | R¹ is 2,5-di-Me-Ph, A is CH₂, X is X² and Y is S. |
| 7-E | R¹ is 2,5-di-Me-Ph, A is NH, X is X² and Y is S. |
| 7-F | R¹ is 3-CF₃-5-Me-1H-pyrazol-1-yl, A is CH₂, X is X¹ and Y is O. |
| 7-G | R¹ is 2,5-di-Me-Ph, A is CH₂, X is X¹ and Y is O. |
| 7-H | R¹ is 2,5-di-Me-Ph, A is NH, X is X¹ and Y is O. |
| 7-I | R¹ is 3-CF₃-5-Me-1H-pyrazol-1-yl, A is CH₂, X is X² and Y is O. |
| 7-J | R¹ is 2,5-di-Me-Ph, A is CH₂, X is X² and Y is O. |
| 7-K | R¹ is 2,5-di-Me-Ph, A is NH, X is X² and Y is O. |
| 7-L | R¹ is 3-CF₃-5-Me-1H-pyrazol-1-yl, A is CH₂, X is X¹ and Y is |
| | NH. |
| 7-M | R¹ is 2,5-di-Me-Ph, A is CH₂, X is X¹ and Y is NH. |
| 7-N | R¹ is 2,5-di-Me-Ph, A is NH, X is X¹ and Y is NH. |
| 7-O | R¹ is 3-CF₃-5-Me-1H-pyrazol-1-yl, A is CH₂, X is X² and Y |
| | is NH. |
| 7-P | R¹ is 2,5-di-Me-Ph, A is CH₂, X is X² and Y is NH. |
| 7-Q | R¹ is 2,5-di-Me-Ph, A is NH, X is X² and Y is NH. |
| 7-R | R¹ is 3-CF₃-5-Me-1H-pyrazol-1-yl, A is CH₂, X is X¹ and Y |
| | is N(Me). |
| 7-S | R¹ is 2,5-di-Me-Ph, A is CH₂, X is X¹ and Y is N(Me). |
| 7-T | R¹ is 2,5-di-Me-Ph, A is NH, X is X¹ and Y is N(Me). |
| 7-U | R¹ is 3-CF₃-5-Me-1H-pyrazol-1-yl, A is CH₂, X is X² and Y |
| | is N(Me). |
| 7-V | R¹ is 2,5-di-Me-Ph, A is CH₂, X is X² and Y is N(Me). |
| 7-W | R¹ is 2,5-di-Me-Ph, A is NH, X is X² and Y is N(Me). |
| 7-X | R¹ is 3,5-bis-(CHF₂)-1H-pyrazol-1-yl, A is CH2, X is X² and Y |
| | is S. |

TABLE 8

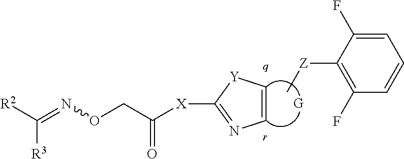

In Table 8 the structures of G-1 through G-30 are
shown in Exhibit 1 above. The substituent R⁸ and is
attached to the G-ring, as defined in the Summary
of the Invention. A dash (“—”) in the R⁸ column below
indicates the G-ring is unsubstituted. The point of
attachment of the G-ring to Z is shown in Exhibit 1 above.

| G | R⁸ | Z | G | R^{7a} | Z | G | R⁸ | Z |
|---|---|---|---|---|---|---|---|---|
| G-1 | — | CH₂ | G-27 | — | CH₂ | G-23 | — | OCH₂CH₂ |
| G-2 | — | CH | G-28 | — | CH₂ | G-24 | — | N(Me) |
| G-3 | — | CH₂ | G-29 | — | CH₂ | G-25 | — | CH₂O |
| G-4 | — | CH₂ | G-30 | — | CH₂ | G-26 | — | NHCH(Me) |
| G-5 | — | CH | G-1 | — | CH₂CH₂ | G-27 | — | OCH₂ |
| G-6 | — | CH₂ | G-2 | — | NNH | G-28 | — | SCH₂ |
| G-7 | — | CH₂ | G-3 | — | CH₂CH₂CH₂ | G-29 | — | O |
| G-8 | — | CH | G-4 | — | CH₂CH₂ | G-30 | — | NH |
| G-9 | — | CH₂ | G-5 | — | CHCH₂ | G-13 | 6-Me | CH₂ |
| G-10 | — | CH₂ | G-6 | — | CH₂CH₂ | G-10 | 4-OH | CH₂ |
| G-11 | — | CH | G-7 | — | CH(OH) | G-10 | 4-Me, | CH₂ |
| G-12 | — | CH₂ | G-8 | — | CH(Me) | | 4-OH | |
| G-13 | — | CH₂ | G-9 | — | CH(Me) | G-13 | — | CH₂OCH₂ |
| G-14 | — | CH | G-10 | — | O | G-13 | — | CH₂SCH₂ |
| G-15 | — | CH₂ | G-11 | — | NO | G-13 | — | CH₂NHCH₂ |
| G-16 | — | CH₂ | G-12 | — | CH(C≡N) | G-13 | — | CH₂N(Me)CH₂ |
| G-17 | — | CH | G-13 | — | CH₂O | G-13 | — | CH₂CH₂O |
| G-18 | — | CH₂ | G-14 | — | CHCH₂ | G-13 | — | CH₂CH₂S |
| G-19 | — | CH₂ | G-15 | — | CH(Me) | G-13 | — | CH₂CH₂N(Me) |
| G-20 | — | CH₂ | G-15 | — | CH(Et) | G-19 | — | SCH₂ |
| G-21 | — | CH₂ | G-13 | — | CH₂S | G-19 | — | NHCH₂ |
| G-22 | — | CH₂ | G-13 | — | CH₂CH₂NH | G-19 | — | OCH₂CH₂ |
| G-23 | — | CH₂ | G-19 | — | OCH₂ | G-19 | — | SCH₂CH₂ |
| G-24 | — | CH₂ | G-20 | — | CH₂O | G-19 | — | CH₂O |
| G-25 | — | CH₂ | G-21 | — | NH | G-19 | — | CH₂S |
| G-26 | — | CH₂ | G-22 | — | S | G-19 | — | CH₂NH |
| | | | | | | G-19 | — | CH₂OCH₂ |
| | | | | | | G-19 | — | CH(OH) |

R² is CF₃, R³ is H, X is X¹ and Y is S.

The present disclosure also includes Tables 8-A through 8-P, each of which is constructed the same as Table 8 above except that the Row Heading in Table 8 (i.e. “R² is CF₃, R³ is H, X is X¹ and Y is S”) is replaced with the respective row headings shown below. For example, in Table 8-A the row heading is “R² is CF₃, R³ is Me, X is X¹ and Y is S” and G, R⁸ and Z are as defined in Table 8 above. Thus, the first entry in Table 8-A specifically discloses 1,1,1-trifluoro-2-propanone O-[2-[4-[5-[(2,6-difluorophenyl)methyl]-5,6-dihydro-4H-cyclopentathiazol-2-yl]-1-piperidinyl]-2-oxoethyl]oxime. Tables 8-B and 8-P are constructed similarly.

| Table | Row Heading |
|---|---|
| 8-A | R² is CF₃, R³ is Me, X is X¹ and Y is S. |
| 8-B | R² is CF₃, R³ is H, X is X² and Y is S. |
| 8-C | R² is CF₃, R³ is Me, X is X² and Y is S. |
| 8-D | R² is CF₃, R³ is H, X is X¹ and Y is O. |
| 8-E | R² is CF₃, R³ is Me, X is X¹ and Y is O. |
| 8-F | R² is CF₃, R³ is H, X is X² and Y is O. |
| 8-G | R² is CF₃, R³ is Me, X is X² and Y is O. |
| 8-H | R² is CF₃, R³ is H, X is X¹ and Y is NH. |
| 8-I | R² is CF₃, R³ is Me, X is X¹ and Y is NH. |
| 8-J | R² is CF₃, R³ is H, X is X² and Y is NH. |
| 8-K | R² is CF₃, R³ is Me, X is X² and Y is NH |
| 8-L | R² is CF₃, R³ is H, X is X¹ and Y is N(Me). |
| 8-M | R² is CF₃, R³ is Me, X is X¹ and Y is N(Me). |
| 8-N | R² is CF₃, R³ is H, X is X² and Y is N(Me). |
| 8-O | R² is CF₃, R³ is Me, X is X² and Y is N(Me). |
| 8-P | R² is CHF₂, R³ is Me, X is X² and Y is S. |

TABLE 9

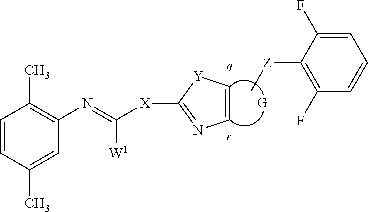

In Table 9 the structures of G-1 through G-30 are
shown in Exhibit 1 above. The substituent R⁸ and is
attached to the G-ring, as defined in the Summary
of the Invention. A dash (“—”) in the R⁸ column below
indicates the G-ring is unsubstituted. The point of
attachment of the G-ring to Z is shown in Exhibit 1 above.

| G | R⁸ | Z | G | R^{7a} | Z | G | R⁸ | Z |
|---|---|---|---|---|---|---|---|---|
| G-1 | — | CH₂ | G-27 | — | CH₂ | G-23 | — | OCH₂CH₂ |
| G-2 | — | CH | G-28 | — | CH₂ | G-24 | — | N(Me) |
| G-3 | — | CH₂ | G-29 | — | CH₂ | G-25 | — | CH₂O |
| G-4 | — | CH₂ | G-30 | — | CH₂ | G-26 | — | NHCH(Me) |
| G-5 | — | CH | G-1 | — | CH₂CH₂ | G-27 | — | OCH₂ |
| G-6 | — | CH₂ | G-2 | — | NNH | G-28 | — | SCH₂ |
| G-7 | — | CH₂ | G-3 | — | CH₂CH₂CH₂ | G-29 | — | O |
| G-8 | — | CH | G-4 | — | CH₂CH₂ | G-30 | — | NH |
| G-9 | — | CH₂ | G-5 | — | CHCH₂ | G-13 | 6-Me | CH₂ |
| G-10 | — | CH₂ | G-6 | — | CH₂CH₂ | G-10 | 4-OH | CH₂ |
| G-11 | — | CH | G-7 | — | CH(OH) | G-10 | 4-Me, | CH₂ |
| G-12 | — | CH₂ | G-8 | — | CH(Me) | | 4-OH | |
| G-13 | — | CH₂ | G-9 | — | CH(Me) | G-13 | — | CH₂OCH₂ |
| G-14 | — | CH | G-10 | — | O | G-13 | — | CH₂SCH₂ |
| G-15 | — | CH₂ | G-11 | — | NO | G-13 | — | CH₂NHCH₂ |
| G-16 | — | CH₂ | G-12 | — | CH(C≡N) | G-13 | — | CH₂N(Me)CH₂ |
| G-17 | — | CH | G-13 | — | CH₂O | G-13 | — | CH₂CH₂O |
| G-18 | — | CH₂ | G-14 | — | CHCH₂ | G-13 | — | CH₂CH₂S |
| G-19 | — | CH₂ | G-15 | — | CH(Me) | G-13 | — | CH₂CH₂N(Me) |
| G-20 | — | CH₂ | G-15 | — | CH(Et) | G-19 | — | SCH₂ |
| G-21 | — | CH₂ | G-13 | — | CH₂S | G-19 | — | NHCH₂ |
| G-22 | — | CH₂ | G-13 | — | CH₂CH₂NH | G-19 | — | OCH₂CH₂ |
| G-23 | — | CH₂ | G-19 | — | OCH₂ | G-19 | — | SCH₂CH₂ |
| G-24 | — | CH₂ | G-20 | — | CH₂O | G-19 | — | CH₂O |
| G-25 | — | CH₂ | G-21 | — | NH | G-19 | — | CH₂S |
| G-26 | — | CH₂ | G-22 | — | S | G-19 | — | CH₂NH |
| | | | | | | G-19 | — | CH₂OCH₂ |
| | | | | | | G-19 | — | CH(OH) |

W¹ is CH₃O, X is X¹ and Y is S.

The present disclosure also includes Tables 9-A through 9-G, each of which is constructed the same as Table 9 above except that the Row Heading in Table 9 (i.e. “W¹ is CH₃O, X is X¹ and Y is S”) is replaced with the respective row headings shown below. For example, in Table 9-A the row heading is “W¹ is CH₃O, X is X² and Y is S” and G, R⁸ and Z are as defined in Table 9 above. Thus, the first entry in Table 9-A specifically discloses methyl 4-[5-[(2,6-difluorophenyl)methyl]-5,6-dihydro-4H-cyclopentathiazol-2-yl]-N-(2,5-dimethylphenyl)-1-piperazinecarboximidate. Tables 9-B and 9-G are constructed similarly.

| Table | Row Heading |
|---|---|
| 9-A | W¹ is CH₃O, X is X² and Y is S. |
| 9-B | W¹ is CH₃O, X is X¹ and Y is O. |
| 9-C | W¹ is CH₃O, X is X² and Y is O. |
| 9-D | W¹ is CH₃O, X is X¹ and Y is NH. |
| 9-E | W¹ is CH₃O, X is X² and Y is NH. |
| 9-F | W¹ is CH₃O, X is X¹ and Y is N(Me). |
| 9-G | W¹ is CH₃O, X is X² and Y is N(Me). |

TABLE 10

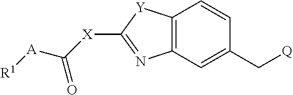

In Table 10 the structures of Q-1 through Q-102 are shown
in Exhibit 2 above. Where applicable, the
substituent R^{10c} on Q is methyl, and p (i.e. in the definition of (R^{10a})p) is 0.
Q
Q-1
Q-2
Q-3
Q-4
Q-5
Q-6
Q-7
Q-8
Q-9
Q-10
Q-11
Q-12
Q-13
Q-14
Q-15
Q-16
Q-17
Q-18
Q-19
Q-20
Q-21
Q-22
Q-23
Q-24
Q-25
Q-26
Q-27
Q-28
Q-29
Q-30
Q-31
Q-32
Q-33
Q-34
Q-35
Q-36
Q-37
Q-38
Q-39
Q-40
Q-41
Q-42
Q-43
Q-44
Q-45
Q-46
Q-47
Q-48
Q-49
Q-50
Q-51
Q-52
Q-53
Q-54
Q-55
Q-56
Q-57
Q-58
Q-59
Q-60
Q-61
Q-62
Q-63
Q-64
Q-65
Q-66
Q-67
Q-68
Q-69
Q-70
Q-71
Q-72
Q-73
Q-74
Q-75
Q-76
Q-77
Q-78
Q-79
Q-80
Q-81
Q-82
Q-83
Q-84
Q-85
Q-86
Q-87
Q-88
Q-89
Q-90
Q-91
Q-92
Q-93
Q-94
Q-95
Q-96
Q-97
Q-98
Q-99
Q-100
Q-101
Q-102
R¹ is 3-CF₃-5-Me-1H-pyrazol-1-yl, A is CH₂, X is X¹ and Y is S.

The present disclosure also includes Tables 10-A through 10-X, each of which is constructed the same as Table 10 above except that the Row Heading in Table 10 (i.e. “R¹ is 3-CF₃-5-Me-1H-pyrazol-1-yl, A is CH₂, X is X¹ and Y is S”) is replaced with the respective row headings shown below. For example, in Table 10-A the row heading is “R¹ is 2,5-di-Me-Ph, A is CH₂, X is X¹ and Y is S” and Q is as defined in Table 10 above. Thus, the first entry in Table 10-A specifically discloses 1-[4-[5-(2-thienylmethyl)-2-benzothiazolyl]-1-piperidinyl]-2-(2,5-dimethylphenyl)ethanone. Tables 10-B and 10-X are constructed similarly.

| Table | Row Heading |
|---|---|
| 10-A | R¹ is 2,5-di-Me-Ph, A is CH₂, X is X¹ and Y is S. |
| 10-B | R¹ is 2,5-di-Me-Ph, A is NH, X is X¹ and Y is S. |
| 10-C | R¹ is 3-CF₃-5-Me-1H-pyrazol-1-yl, A is CH₂, X is X² and Y is S. |
| 10-D | R¹ is 2,5-di-Me-Ph, A is CH₂, X is X² and Y is S. |
| 10-E | R¹ is 2,5-di-Me-Ph, A is NH, X is X² and Y is S. |
| 10-F | R¹ is 3-CF₃-5-Me-1H-pyrazol-1-yl, A is CH₂, X is X¹ and Y is O. |
| 10-G | R¹ is 2,5-di-Me-Ph, A is CH₂, X is X¹ and Y is O. |
| 10-H | R¹ is 2,5-di-Me-Ph, A is NH, X is X¹ and Y is O. |
| 10-I | R¹ is 3-CF₃-5-Me-1H-pyrazol-1-yl, A is CH₂, X is X² and Y is O. |
| 10-J | R¹ is 2,5-di-Me-Ph, A is CH₂, X is X² and Y is O. |
| 10-K | R¹ is 2,5-di-Me-Ph, A is NH, X is X² and Y is O. |
| 10-L | R¹ is 3-CF₃-5-Me-1H-pyrazol-1-yl, A is CH₂, X is X¹ and Y is |
| | NH. |
| 10-M | R¹ is 2,5-di-Me-Ph, A is CH₂, X is X¹ and Y is NH. |
| 10-N | R¹ is 2,5-di-Me-Ph, A is NH, X is X¹ and Y is NH. |
| 10-O | R¹ is 3-CF₃-5-Me-1H-pyrazol-1-yl, A is CH₂, X is X² and Y is |
| | NH. |
| 10-P | R¹ is 2,5-di-Me-Ph, A is CH₂, X is X² and Y is NH. |
| 10-Q | R¹ is 3-CF₃-5-Me-1H-pyrazol-1-yl, A is CH₂, X is X¹ and Y is |
| | N(Me). |
| 10-R | R¹ is 2,5-di-Me-Ph, A is CH₂, X is X¹ and Y is N(Me). |
| 10-S | R¹ is 2,5-di-Me-Ph, A is NH, X is X¹ and Y is N(Me). |
| 10-T | R¹ is 3-CF₃-5-Me-1H-pyrazol-1-yl, A is CH₂, X is X² and Y is |
| | N(Me). |
| 10-U | R¹ is 2,5-di-Me-Ph, A is CH₂, X is X² and Y is N(Me). |
| 10-V | R¹ is 2,5-di-Me-Ph, A is NH, X is X² and Y is N(Me). |
| 10-W | R¹ is 3,5-bis-(CHF₂)-1H-pyrazol-1-yl, A is CH₂, X is X¹ and Y |
| | is S. |
| 10-X | R¹ is 3,5-bis-(CHF₂)-1H-pyrazol-1-yl, A is CH₂, X is X¹ and Y |
| | is O. |

TABLE 11

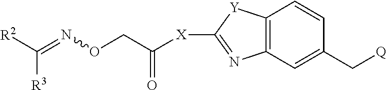

In Table 11 the structures of Q-1 through Q-102 are shown
in Exhibit 2 above. Where applicable, the
substituent R^{10c} on Q is methyl, and p (i.e. in the definition of (R^{10a})p) is 0.
Q
Q-1
Q-2
Q-3
Q-4
Q-5
Q-6
Q-7
Q-8
Q-9
Q-10
Q-11
Q-12
Q-13
Q-14
Q-15
Q-16
Q-17
Q-18
Q-19
Q-20
Q-21
Q-22
Q-23
Q-24
Q-25
Q-26
Q-27
Q-28
Q-29
Q-30
Q-31
Q-32
Q-33
Q-34
Q-35
Q-36
Q-37
Q-38
Q-39
Q-40
Q-41
Q-42
Q-43
Q-44
Q-45
Q-46
Q-47
Q-48
Q-49
Q-50
Q-51
Q-52
Q-53
Q-54
Q-55
Q-56
Q-57
Q-58
Q-59
Q-60
Q-61
Q-62
Q-63
Q-64
Q-65
Q-66
Q-67
Q-68
Q-69
Q-70
Q-71
Q-72
Q-73
Q-74
Q-75
Q-76
Q-77
Q-78
Q-79
Q-80
Q-81
Q-82
Q-83
Q-84
Q-85
Q-86
Q-87
Q-88
Q-89
Q-90
Q-91
Q-92
Q-93
Q-94
Q-95
Q-96
Q-97
Q-98
Q-99
Q-100
Q-101
Q-102
R² is CF₃, R³ is H, X is X1 and Y is S.

The present disclosure also includes Tables 11-A through 11-Q, each of which is constructed the same as Table 11 above except that the Row Heading in Table 11 (i.e. “R² is CF₃, R³ is H, X is X¹ and Y is S”) is replaced with the respective row headings shown below. For example, in Table 11-A the row heading is “R² is CF₃, R³ is Me, X is X¹ and Y is S” and Q is as defined in Table 11 above. Thus, the first entry in Table 11-A specifically discloses 1,1,1-trifluoro-2-propanone O-[2-oxo-2-[4-[5-(2-thienylmethyl)-2-benzothiazolyl]-1-piperidinyl]ethyl]oxime. Tables 11-B and 11-Q are constructed similarly.

| Table | Row Heading |
|---|---|
| 11-A | R² is CF₃, R³ is Me, X is X¹ and Y is S. |
| 11-B | R² is CF₃, R³ is H, X is X² and Y is S. |
| 11-C | R² is CF₃, R³ is Me, X is X² and Y is S. |
| 11-D | R² is CF₃, R³ is H, X is X¹ and Y is O. |
| 11-E | R² is CF₃, R³ is Me, X is X¹ and Y is O. |
| 11-F | R² is CF₃, R³ is H, X is X² and Y is O. |
| 11-G | R² is CF₃, R³ is Me, X is X² and Y is O. |
| 11-H | R² is CF₃, R³ is H, X is X¹ and Y is NH. |
| 11-I | R² is CF₃, R³ is Me, X is X¹ and Y is NH. |
| 11-J | R² is CF₃, R³ is H, X is X² and Y is NH. |
| 11-K | R² is CF₃, R³ is Me, X is X² and Y is NH |
| 11-L | R² is CF₃, R³ is H, X is X¹ and Y is N(Me). |
| 11-M | R² is CF₃, R³ is Me, X is X¹ and Y is N(Me). |
| 11-N | R² is CF₃, R³ is H, X is X² and Y is N(Me). |
| 11-O | R² is CF₃, R³ is Me, X is X² and Y is N(Me). |
| 11-P | R² is CHF₂, R³ is Me, X is X¹ and Y is S. |
| 11-Q | R² is CHF₂, R³ is Me, X is X¹ and Y is O. |

TABLE 12

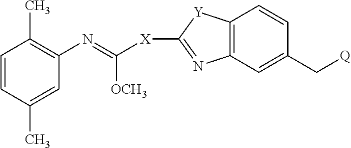

In Table 12 the structures of Q-1 through Q-102 are shown
in Exhibit 2 above. Where applicable, the
substituent R^{10c} on Q is methyl, and p (i.e. in the definition of (R^{10a})p) is 0.
Q
Q-1
Q-2
Q-3
Q-4
Q-5
Q-6
Q-7
Q-8
Q-9
Q-10
Q-11
Q-12
Q-13
Q-14
Q-15
Q-16
Q-17
Q-18
Q-19
Q-20
Q-21
Q-22
Q-23
Q-24
Q-25
Q-26
Q-27
Q-28
Q-29
Q-30
Q-31
Q-32
Q-33
Q-34
Q-35
Q-36
Q-37
Q-38
Q-39
Q-40
Q-41
Q-42
Q-43
Q-44
Q-45
Q-46
Q-47
Q-48
Q-49
Q-50
Q-51
Q-52
Q-53
Q-54
Q-55
Q-56
Q-57
Q-58
Q-59
Q-60
Q-61
Q-62
Q-63
Q-64
Q-65
Q-66
Q-67
Q-68
Q-69
Q-70
Q-71
Q-72
Q-73
Q-74
Q-75
Q-76
Q-77
Q-78
Q-79
Q-80
Q-81
Q-82
Q-83
Q-84
Q-85
Q-86
Q-87
Q-88
Q-89
Q-90
Q-91
Q-92
Q-93
Q-94
Q-95
Q-96
Q-97
Q-98
Q-99
Q-100
Q-101
Q-102
X is X¹ and Y is S.

The present disclosure also includes Tables 12-A through 12-H, each of which is constructed the same as Table 12 above except that the Row Heading in Table 12 (i.e. “X is X¹ and Y is S.”) is replaced with the respective row headings shown below. For example, in Table 12-A the row heading is “X is X² and Y is S” and Q is as defined in Table 12 above. Thus, the first entry in Table 12-A specifically discloses methyl N-(2,5-dimethylphenyl)-4-[5-(2-thienylmethyl)-2-benzothiazolyl]-1-piperazinecarboximidate. Tables 12-B and 12-H are constructed similarly.

| Table | Row Heading |
|---|---|
| 12-A | X is X² and Y is S. |
| 12-B | X is X¹ and Y is O. |
| 12-C | X is X² and Y is O. |
| 12-D | X is X¹ and Y is NH. |
| 12-E | X is X² and Y is NH. |
| 12-F | X is X² and Y is NH. |
| 12-G | X is X¹ and Y is N(Me). |
| 12-H | X is X² and Y is N(Me). |

### Formulation/Utility

A compound of this invention will generally be used as a fungicidal active ingredient in a composition, i.e. formulation, with at least one additional component selected from the group consisting of surfactants, solid diluents and liquid diluents, which serve as a carrier. The formulation or composition ingredients are selected to be consistent with the physical properties of the active ingredient, mode of application and environmental factors such as soil type, moisture and temperature.

Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspo-emulsion. The general types of nonaqueous liquid compositions are emulsifiable concentrate, microemulsifiable concentrate, dispersible concentrate and oil dispersion.

The general types of solid compositions are dusts, powders, granules, pellets, prills, pastilles, tablets, filled films (including seed coatings) and the like, which can be water-dispersible (“wettable”) or water-soluble. Films and coatings formed from film-forming solutions or flowable suspensions are particularly useful for seed treatment. Active ingredient can be (micro)encapsulated and further formed into a suspension or solid formulation; alternatively the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions are primarily used as intermediates for further formulation.

Sprayable formulations are typically extended in a suitable medium before spraying. Such liquid and solid formulations are formulated to be readily diluted in the spray medium, usually water. Spray volumes can range from about one to several thousand liters per hectare, but more typically are in the range from about ten to several hundred liters per hectare. Sprayable formulations can be tank mixed with water or another suitable medium for foliar treatment by aerial or ground application, or for application to the growing medium of the plant. Liquid and dry formulations can be metered directly into drip irrigation systems or metered into the furrow during planting. Liquid and solid formulations can be applied onto seeds of crops and other desirable vegetation as seed treatments before planting to protect developing roots and other subterranean plant parts and/or foliage through systemic uptake.

The formulations will typically contain effective amounts of active ingredient, diluent and surfactant within the following approximate ranges which add up to 100 percent by weight.

| | Weight Percent | | |
|---|---|---|---|
| | Active | | |
| | Ingredient | Diluent | Surfactant |
| Water-Dispersible and | 0.001-90 | 0-99.999 | 0-15 |
| Water-soluble Granules, | | | |
| Tablets and Powders | | | |
| Oil Dispersions, | 1-50 | 40-99 | 0-50 |
| Suspensions, Emulsions, | | | |
| Solutions (including | | | |
| Emulsifiable Concentrates) | | | |
| Dusts | 1-25 | 70-99 | 0-5 |
| Granules and Pellets | 0.001-95 | 5-99.999 | 0-15 |
| High Strength | 90-99 | 0-10 | 0-2 |
| Compositions | | | |

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and y-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof. Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950.

The solid and liquid compositions of the present invention often include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents.

Surfactants can be classified as nonionic, anionic or cationic. Nonionic surfactants useful for the present compositions include, but are not limited to: alcohol alkoxylates such as alcohol alkoxylates based on natural and synthetic alcohols (which may be branched or linear) and prepared from the alcohols and ethylene oxide, propylene oxide, butylene oxide or mixtures thereof; amine ethoxylates, alkanolamides and ethoxylated alkanolamides; alkoxylated triglycerides such as ethoxylated soybean, castor and rapeseed oils; alkylphenol alkoxylates such as octylphenol ethoxylates, nonylphenol ethoxylates, dinonyl phenol ethoxylates and dodecyl phenol ethoxylates (prepared from the phenols and ethylene oxide, propylene oxide, butylene oxide or mixtures thereof); block polymers prepared from ethylene oxide or propylene oxide and reverse block polymers where the terminal blocks are prepared from propylene oxide; ethoxylated fatty acids; ethoxylated fatty esters and oils; ethoxylated methyl esters; ethoxylated tristyrylphenol (including those prepared from ethylene oxide, propylene oxide, butylene oxide or mixtures thereof); fatty acid esters, glycerol esters, lanolin-based derivatives, polyethoxylate esters such as polyethoxylated sorbitan fatty acid esters, polyethoxylated sorbitol fatty acid esters and polyethoxylated glycerol fatty acid esters; other sorbitan derivatives such as sorbitan esters; polymeric surfactants such as random copolymers, block copolymers, alkyd peg (polyethylene glycol) resins, graft or comb polymers and star polymers; polyethylene glycols (pegs); polyethylene glycol fatty acid esters; silicone-based surfactants; and sugar-derivatives such as sucrose esters, alkyl polyglycosides and alkyl polysaccharides.

Useful anionic surfactants include, but are not limited to: alkylaryl sulfonic acids and their salts; carboxylated alcohol or alkylphenol ethoxylates; diphenyl sulfonate derivatives; lignin and lignin derivatives such as lignosulfonates; maleic or succinic acids or their anhydrides; olefin sulfonates; phosphate esters such as phosphate esters of alcohol alkoxylates, phosphate esters of alkylphenol alkoxylates and phosphate esters of styryl phenol ethoxylates; protein-based surfactants; sarcosine derivatives; styryl phenol ether sulfate; sulfates and sulfonates of oils and fatty acids; sulfates and sulfonates of ethoxylated alkylphenols; sulfates of alcohols; sulfates of ethoxylated alcohols; sulfonates of amines and amides such as N,N-alkyltaurates; sulfonates of benzene, cumene, toluene, xylene, and dodecyl and tridecylbenzenes; sulfonates of condensed naphthalenes; sulfonates of naphthalene and alkyl naphthalene; sulfonates of fractionated petroleum; sulfosuccinamates; and sulfosuccinates and their derivatives such as dialkyl sulfosuccinate salts.

Useful cationic surfactants include, but are not limited to: amides and ethoxylated amides; amines such as N-alkyl propanediamines, tripropylenetriamines and dipropylenetetramines, and ethoxylated amines, ethoxylated diamines and propoxylated amines (prepared from the amines and ethylene oxide, propylene oxide, butylene oxide or mixtures thereof); amine salts such as amine acetates and diamine salts; quaternary ammonium salts such as quaternary salts, ethoxylated quaternary salts and diquaternary salts; and amine oxides such as alkyldimethylamine oxides and bis-(2-hydroxyethyl)-alkylamine oxides.

Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co.; and PCT Patent Publication WO 03/024222.

The compound of Formula 1 and any other active ingredients are typically incorporated into the present compositions by dissolving the active ingredient in a solvent or by grinding in a liquid or dry diluent. Solutions, including emulsifiable concentrates, can be prepared by simply mixing the ingredients. If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. No. 4,144,050, U.S. Pat. No. 3,920,442 and DE 3,246,493. Tablets can be prepared as taught in U.S. Pat. No. 5,180,587, U.S. Pat. No. 5,232,701 and U.S. Pat. No. 5,208,030. Films can be prepared as taught in GB 2,095,558 and U.S. Pat. No. 3,299,566.

For further information regarding the art of formulation, see T. S. Woods, “The Formulator's Toolbox—Product Forms for Modern Agriculture” in Pesticide Chemistry and Bioscience, The Food—Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, UK, 2000.

In the following Examples, all percentages are by weight and all formulations are prepared in conventional ways. Compound numbers refer to compounds in Index Tables A-B. Without further elaboration, it is believed that one skilled in the art using the preceding description can utilize the present invention to its fullest extent. The following Examples are, therefore, to be constructed as merely illustrative, and not limiting of the disclosure in any way whatsoever. Percentages are by weight except where otherwise indicated.

### Example A

High Strength Concentrate

| | |
|---|---|
| Compound 8 | 98.5% |
| silica aerogel | 0.5% |
| synthetic amorphous fine silica | 1.0% |

### Example B

Wettable Powder

| | |
|---|---|
| Compound 5 | 65.0% |
| dodecylphenol polyethylene glycol ether | 2.0% |
| sodium ligninsulfonate | 4.0% |
| sodium silicoaluminate | 6.0% |
| montmorillonite (calcined) | 23.0% |

### Example C

Grandule

| | |
|---|---|
| Compound 8 | 10.0% |
| attapulgite granules (low volatile matter, | 90.0% |
| 0.71/0.30 mm; U.S.S. No. 25-50 sieves) | |

### Example D

Extruded Pellet

| | |
|---|---|
| Compound 6 | 25.0% |
| anhydrous sodium sulfate | 10.0% |
| crude calcium ligninsulfonate | 5.0% |
| sodium alkylnaphthalenesulfonate | 1.0% |
| calcium/magnesium bentonite | 59.0% |

### Example E

Emulsifiable Concentrate

| | |
|---|---|
| Compound 8 | 10.0% |
| polyoxyethylene sorbitol hexoleate | 20.0% |
| C₆-C₁₀ fatty acid methyl ester | 70.0% |

### Example F

Microemulsion

| | |
|---|---|
| Compound 5 | 5.0% |
| polyvinylpyrrolidone-vinyl acetate copolymer | 30.0% |
| alkylpolyglycoside | 30.0% |
| glyceryl monooleate | 15.0% |
| water | 20.0% |

### Example G

Seed Treatment

| | |
|---|---|
| Compound 6 | 20.00% |
| polyvinylpyrrolidone-vinyl acetate copolymer | 5.00% |
| montan acid wax | 5.00% |
| calcium ligninsulfonate | 1.00% |
| polyoxyethylene/polyoxypropylene block copolymers | 1.00% |
| stearyl alcohol (POE 20) | 2.00% |
| polyorganosilane | 0.20% |
| colorant red dye | 0.05% |
| water | 65.75% |

Water-soluble and water-dispersible formulations are typically diluted with water to form aqueous compositions before application. Aqueous compositions for direct applications to the plant or portion thereof (e.g., spray tank compositions) typically at least about 1 ppm or more (e.g., from 1 ppm to 100 ppm) of the compound(s) of this invention.

The compounds of this invention are useful as plant disease control agents. The present invention therefore further comprises a method for controlling plant diseases caused by fungal plant pathogens comprising applying to the plant or portion thereof to be protected, or to the plant seed to be protected, an effective amount of a compound of the invention or a fungicidal composition containing said compound. The compounds and/or compositions of this invention provide control of diseases caused by a broad spectrum of fungal plant pathogens in the Basidiomycete, Ascomycete, Oomycete and Deuteromycete classes. They are effective in controlling a broad spectrum of plant diseases, particularly foliar pathogens of ornamental, turf, vegetable, field, cereal, and fruit crops. These pathogens include: Oomycetes, including Phytophthora diseases such as Phytophthora infestans, Phytophthora megasperma, Phytophthora parasitica, Phytophthora cinnamomi and Phytophthora capsici, Pythium diseases such as Pythium aphanidermatum, and diseases in the Peronosporaceae family such as Plasmopara viticola, Peronospora spp. (including Peronospora tabacina and Peronospora parasitica), Pseudoperonospora spp. (including Pseudoperonospora cubensis) and Bremia lactucae; Ascomycetes, including Alternaria diseases such as Alternaria solani and Alternaria brassicae, Guignardia diseases such as Guignardia bidwell, Venturia diseases such as Venturia inaequalis, Septoria diseases such as Septoria nodorum and Septoria tritici, powdery mildew diseases such as Erysiphe spp. (including Erysiphe graminis and Erysiphe polygoni), Uncinula necatur, Sphaerotheca fuliginea, Podosphaera leucotricha and Pseudocercosporella herpotrichoides, Botrytis diseases such as Botrytis cinerea, Monilinia fructicola, Sclerotinia diseases such as Sclerotinia sclerotiorum, Sclerotinia minor, Magnaporthe grisea, and Phomopsis viticola, Helminthosporium diseases such as Helminthosporium tritici repentis and Pyrenophora teres, anthracnose diseases such as Glomerella or Colletotrichum spp. (such as Colletotrichum graminicola and Colletotrichum orbiculare), and Gaeumannomyces graminis; Basidiomycetes, including rust diseases caused by Puccinia spp. (such as Puccinia recondite, Puccinia striiformis, Puccinia hordei, Puccinia graminis and Puccinia arachidis), Hemileia vastatrix and Phakopsora pachyrhizi; other pathogens including Rutstroemia floccosum (also known as Sclerontina homoeocarpa); Rhizoctonia spp. (such as Rhizoctonia solani); Fusarium diseases such as Fusarium roseum, Fusarium graminearum and Fusarium oxysporum Verticillium dahliae; Sclerotium rolfsii; Rynchosporium secalis; Cercosporidium personatum, Cercospora arachidicola and Cercospora beticola; Rhizopus spp. (such as Rhizopus stolnifer); Aspergillus spp. (such as Aspergillus flavus and Aspergillus parasiticus); and other genera and species closely related to these pathogens. In addition to their fungicidal activity, the compositions or combinations also have activity against bacteria such as Erwinia amylovora, Xanthomonas campestris, Pseudomonas syringae, and other related species. Furthermore, the compounds of this invention are useful in treating postharvest diseases of fruits and vegetables caused by fungi and bacteria. These infections can occur before, during and after harvest. For example, infections can occur before harvest and then remain dormant until some point during ripening (e.g., host begins tissue changes in such a way that infection can progress); also infections can arise from surface wounds created by mechanical or insect injury. In this respect, the compounds of this invention can reduce losses (i.e. losses resulting from quantity and quality) due to postharvest diseases which may occur at any time from harvest to consumption. Treatment of postharvest diseases with compounds of the invention can increase the period of time during which perishable edible plant parts (e.g., fruits, seeds, foliage, stems, bulbs. tubers) can be stored refrigerated or un-refrigerated after harvest, and remain edible and free from noticeable or harmful degradation or contamination by fungi or other microorganisms. Treatment of edible plant parts before or after harvest with compounds of the invention can also decrease the formation of toxic metabolites of fungi or other microorganisms, for example mycotoxins such as aflatoxins.

Plant disease control is ordinarily accomplished by applying an effective amount of a compound of this invention either pre- or post-infection, to the portion of the plant to be protected such as the roots, stems, foliage, fruits, seeds, tubers or bulbs, or to the media (soil or sand) in which the plants to be protected are growing. The compounds can also be applied to seeds to protect the seeds and seedlings developing from the seeds. The compounds can also be applied through irrigation water to treat plants. Control of postharvest pathogens which infect the produce before harvest is typically accomplished by field application of a compound of this invention, and in cases where infection occurs after harvest the compounds can be applied to the harvested crop as dips, sprays, fumigants, treated wraps and box liners.

Rates of application for these compounds (i.e. a fungicidally effective amount) can be influenced by factors such as the plant diseases to be controlled, the plant species to be protected, ambient moisture and temperature and should be determined under actual use conditions. One skilled in the art can easily determine through simple experimentation the fungicidally effective amount necessary for the desired level of plant disease control. Foliage can normally be protected when treated at a rate of from less than about 1 g/ha to about 5,000 g/ha of active ingredient. Seed and seedlings can normally be protected when seed is treated at a rate of from about 0.1 to about 10 g per kilogram of seed.

Compounds of this invention can also be mixed with one or more other biologically active compounds or agents including fungicides, insecticides, nematocides, bactericides, acaricides, herbicides, herbicide safeners, growth regulators such as insect molting inhibitors and rooting stimulants, chemosterilants, semiochemicals, repellents, attractants, pheromones, feeding stimulants, plant nutrients, other biologically active compounds or entomopathogenic bacteria, virus or fungi to form a multi-component pesticide giving an even broader spectrum of agricultural protection. Thus the present invention also pertains to a composition comprising a compound of Formula 1 (in a fungicidally effective amount) and at least one additional biologically active compound or agent (in a biologically effective amount) and can further comprise at least one of a surfactant, a solid diluent or a liquid diluent. The other biologically active compounds or agents can be formulated in compositions comprising at least one of a surfactant, solid or liquid diluent. For mixtures of the present invention, one or more other biologically active compounds or agents can be formulated together with a compound of Formula 1, to form a premix, or one or more other biologically active compounds or agents can be formulated separately from the compound of Formula 1, and the formulations combined together before application (e.g., in a spray tank) or, alternatively, applied in succession.

Of note is a composition which in addition to the compound of Formula 1 include at least one fungicidal compound selected from the group consisting of the classes (1) methyl benzimidazole carbamate (MBC) fungicides; (2) dicarboximide fungicides; (3) demethylation inhibitor (DMI) fungicides; (4) phenylamide fungicides; (5) amine/morpholine fungicides; (6) phospholipid biosynthesis inhibitor fungicides; (7) carboxamide fungicides; (8) hydroxy(2-amino-)pyrimidine fungicides; (9) anilinopyrimidine fungicides; (10) N-phenyl carbamate fungicides; (11) quinone outside inhibitor (QoI) fungicides; (12) phenylpyrrole fungicides; (13) quinoline fungicides; (14) lipid peroxidation inhibitor fungicides; (15) melanin biosynthesis inhibitors-reductase (MBI-R) fungicides; (16) melanin biosynthesis inhibitors-dehydratase (MBI-D) fungicides; (17) hydroxyanilide fungicides; (18) squalene-epoxidase inhibitor fungicides; (19) polyoxin fungicides; (20) phenylurea fungicides; (21) quinone inside inhibitor (QiI) fungicides; (22) benzamide fungicides; (23) enopyranuronic acid antibiotic fungicides; (24) hexopyranosyl antibiotic fungicides; (25) glucopyranosyl antibiotic: protein synthesis fungicides; (26) glucopyranosyl antibiotic: trehalase and inositol biosynthesis fungicides; (27) cyanoacetamideoxime fungicides; (28) carbamate fungicides; (29) oxidative phosphorylation uncoupling fungicides; (30) organo tin fungicides; (31) carboxylic acid fungicides; (32) heteroaromatic fungicides; (33) phosphonate fungicides; (34) phthalamic acid fungicides; (35) benzotriazine fungicides; (36) benzene-sulfonamide fungicides; (37) pyridazinone fungicides; (38) thiophene-carboxamide fungicides; (39) pyrimidinamide fungicides; (40) carboxylic acid amide (CAA) fungicides; (41) tetracycline antibiotic fungicides; (42) thiocarbamate fungicides; (43) benzamide fungicides; (44) host plant defense induction fungicides; (45) multi-site contact activity fungicides; (46) fungicides other than classes (1) through (45); and salts of compounds of classes (1) through (46).

Further descriptions of these classes of fungicidal compounds are provided below.

(1) “Methyl benzimidazole carbamate (MBC) fungicides” (Fungicide Resistance Action Committee (FRAC) code 1) inhibit mitosis by binding to β-tubulin during microtubule assembly. Inhibition of microtubule assembly can disrupt cell division, transport within the cell and cell structure. Methyl benzimidazole carbamate fungicides include benzimidazoles and thiophanates. The benzimidazoles include benomyl, carbendazim, fuberidazole and thiabendazole. The thiophanates include thiophanate and thiophanate-methyl.

(2) “Dicarboximide fungicides” (Fungicide Resistance Action Committee (FRAC) code 2) are proposed to inhibit a lipid peroxidation in fungi through interference with NADH cytochrome c reductase. Examples include chlozolinate, iprodione, procymidone and vinclozolin.

(3) “Demethylation inhibitor (DMI) fungicides” (Fungicide Resistance Action Committee (FRAC) code 3) inhibit C14-demethylase, which plays a role in sterol production. Sterols, such as ergosterol, are needed for membrane structure and function, making them essential for the development of functional cell walls. Therefore, exposure to these fungicides results in abnormal growth and eventually death of sensitive fungi. Demethylation fungicides include azoles (including triazoles and imidazoles), pyrimidines, piperazines and pyridines. The triazoles include azaconazole, bitertanol, bromuconazole, cyproconazole, difenoconazole, diniconazole (including diniconazole-M), epoxiconazole, fenbuconazole, fluquinconazole, flusilazole, flutriafol, hexaconazole, imibenconazole, ipconazole, metconazole, myclobutanil, penconazole, propiconazole, prothioconazole, simeconazole, tebuconazole, tetraconazole, triadimefon, triadimenol, triticonazole and uniconazole. The imidazoles include clotrimazole, imazalil, oxpoconazole, prochloraz, pefurazoate and triflumizole. The pyrimidines include fenarimol and nuarimol. The piperazines include triforine. The pyridines include pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

(4) “Phenylamide fungicides” (Fungicide Resistance Action Committee (FRAC) code 4) are specific inhibitors of RNA polymerase in Oomycete fungi. Sensitive fungi exposed to these fungicides show a reduced capacity to incorporate uridine into rRNA. Growth and development in sensitive fungi is prevented by exposure to this class of fungicide. Phenylamide fungicides include acylalanines, oxazolidinones and butyrolactones. The acylalanines include benalaxyl, benalaxyl-M, furalaxyl, metalaxyl and metalaxyl-M/mefenoxam. The oxazolidinones include oxadixyl. The butyrolactones include ofurace.

(5) “Amine/morpholine fungicides” (Fungicide Resistance Action Committee (FRAC) code 5) inhibit two target sites within the sterol biosynthetic pathway, Δ⁸→Δ⁷ isomerase and Δ¹⁴ reductase. Sterols, such as ergosterol, are needed for membrane structure and function, making them essential for the development of functional cell walls. Therefore, exposure to these fungicides results in abnormal growth and eventually death of sensitive fungi. Amine/morpholine fungicides (also known as non-DMI sterol biosynthesis inhibitors) include morpholines, piperidines and spiroketal-amines. The morpholines include aldimorph, dodemorph, fenpropimorph, tridemorph and trimorphamide. The piperidines include fenpropidin and piperalin. The spiroketal-amines include spiroxamine.

(6) “Phospholipid biosynthesis inhibitor fungicides” (Fungicide Resistance Action Committee (FRAC) code 6) inhibit growth of fungi by affecting phospholipid biosynthesis. Phospholipid biosynthesis fungicides include phophorothiolates and dithiolanes. The phosphorothiolates include edifenphos, iprobenfos and pyrazophos. The dithiolanes include isoprothiolane.

(7) “Carboxamide fungicides” (Fungicide Resistance Action Committee (FRAC) code 7) inhibit Complex II (succinate dehydrogenase) fungal respiration by disrupting a key enzyme in the Krebs Cycle (TCA cycle) named succinate dehydrogenase. Inhibiting respiration prevents the fungus from making ATP, and thus inhibits growth and reproduction. Carboxamide fungicides include benzamides, furan carboxamides, oxathiin carboxamides, thiazole carboxamides, pyrazole carboxamides, pyridine carboxamides and thiophene carboxamides. The benzamides include benodanil, flutolanil and mepronil. The furan carboxamides include fenfuram. The oxathiin carboxamides include carboxin and oxycarboxin. The thiazole carboxamides include thifluzamide. The pyrazole carboxamides include furametpyr, penthiopyrad, bixafen, isopyrazam, benzovindiflupyr, N-[2-(1S,2R)-[1,1′-bicyclopropyl]-2-ylphenyl]-3-(difluoromethyl)-1-methyl-1H-pyrazole-4-carboxamide, penflufen, (N-[2-(1,3-dimethylbutyl)phenyl]-5-fluoro-1,3-dimethyl-1H-pyrazole-4-carboxamide) and N-[2-(2,4-dichlorophenyl)-2-methoxy-1-methylethyl]-3-(difluoromethyl)-1-methyl-1H-pyrazole-4-carboxamide. The pyridine carboxamides include boscalid. The thiophene carboxamides include isofetamid.

(8) “Hydroxy(2-amino-)pyrimidine fungicides” (Fungicide Resistance Action Committee (FRAC) code 8) inhibit nucleic acid synthesis by interfering with adenosine deaminase. Examples include bupirimate, dimethirimol and ethirimol.
(9) “Anilinopyrimidine fungicides” (Fungicide Resistance Action Committee (FRAC) code 9) are proposed to inhibit biosynthesis of the amino acid methionine and to disrupt the secretion of hydrolytic enzymes that lyse plant cells during infection. Examples include cyprodinil, mepanipyrim and pyrimethanil.

(10) “N-Phenyl carbamate fungicides” (Fungicide Resistance Action Committee (FRAC) code 10) inhibit mitosis by binding to β-tubulin and disrupting microtubule assembly. Inhibition of microtubule assembly can disrupt cell division, transport within the cell and cell structure. Examples include diethofencarb.

(11) “Quinone outside inhibitor (QoI) fungicides” (Fungicide Resistance Action Committee (FRAC) code 11) inhibit Complex III mitochondrial respiration in fungi by affecting ubiquinol oxidase. Oxidation of ubiquinol is blocked at the “quinone outside” (Qₒ) site of the cytochrome bc₁ complex, which is located in the inner mitochondrial membrane of fungi. Inhibiting mitochondrial respiration prevents normal fungal growth and development. Quinone outside inhibitor fungicides (also known as strobilurin fungicides) include methoxyacrylates, methoxycarbamates, oximinoacetates, oximinoacetamides, oxazolidinediones, dihydrodioxazines, imidazolinones and benzylcarbamates. The methoxyacrylates include azoxystrobin, enestroburin (SYP-Z071), picoxystrobin and pyraoxystrobin (SYP-3343) . The methoxycarbamates include pyraclostrobin and pyrametostrobin (SYP-4155). The oximinoacetates include kresoxim-methyl and trifloxystrobin. The oximinoacetamides include dimoxystrobin, metominostrobin, orysastrobin, α-[methoxyimino]-N-methyl-2-[[[1-[3-(trifluoromethyl)phenyl]ethoxy]imino]-methyl]benzeneacetamide and 2-[[[3-(2,6-dichlorophenyl)-1-methyl-2-propen-1-ylidene]-amino]oxy]methyl]-α-(methoxyimino)-N-methylbenzeneacetamide. The oxazolidinediones include famoxadone. The dihydrodioxazines include fluoxastrobin. The imidazolinones include fenamidone. The benzylcarbamates include pyribencarb. Class (11) also includes 2-[(2,5-dimethylphenoxy)methyl]-a-methoxy-N-benzeneacetamide.

(12) “Phenylpyrrole fungicides” (Fungicide Resistance Action Committee (FRAC) code 12) inhibit a MAP protein kinase associated with osmotic signal transduction in fungi. Fenpiclonil and fludioxonil are examples of this fungicide class.

(13) “Quinoline fungicides” (Fungicide Resistance Action Committee (FRAC) code 13) are proposed to inhibit signal transduction by affecting G-proteins in early cell signaling. They have been shown to interfere with germination and/or appressorium formation in fungi that cause powder mildew diseases. Quinoxyfen and tebufloquin are examples of this class of fungicide.

(14) “Lipid peroxidation inhibitor fungicides” (Fungicide Resistance Action Committee (FRAC) code 14) are proposed to inhibit lipid peroxidation which affects membrane synthesis in fungi. Members of this class, such as etridiazole, may also affect other biological processes such as respiration and melanin biosynthesis. Lipid peroxidation fungicides include aromatic carbons and 1,2,4-thiadiazoles. The aromatic carbon fungicides include biphenyl, chloroneb, dicloran, quintozene, tecnazene and tolclofos-methyl. The 1,2,4-thiadiazole fungicides include etridiazole.

(15) “Melanin biosynthesis inhibitors-reductase (MBI-R) fungicides” (Fungicide Resistance Action Committee (FRAC) code 16.1) inhibit the naphthal reduction step in melanin biosynthesis. Melanin is required for host plant infection by some fungi. Melanin biosynthesis inhibitors-reductase fungicides include isobenzofuranones, pyrroloquinolinones and triazolobenzothiazoles. The isobenzofuranones include fthalide. The pyrroloquinolinones include pyroquilon. The triazolobenzothiazoles include tricyclazole.

(16) “Melanin biosynthesis inhibitors-dehydratase (MBI-D) fungicides” (Fungicide Resistance Action Committee (FRAC) code 16.2) inhibit scytalone dehydratase in melanin biosynthesis. Melanin in required for host plant infection by some fungi. Melanin biosynthesis inhibitors-dehydratase fungicides include cyclopropanecarboxamides, carboxamides and propionamides. The cyclopropanecarboxamides include carpropamid. The carboxamides include diclocymet. The propionamides include fenoxanil.

(17) “Hydroxyanilide fungicides (Fungicide Resistance Action Committee (FRAC) code 17) inhibit C4-demethylase which plays a role in sterol production. Examples include fenhexamid.

(18) “Squalene-epoxidase inhibitor fungicides” (Fungicide Resistance Action Committee (FRAC) code 18) inhibit squalene-epoxidase in ergosterol biosynthesis pathway. Sterols such as ergosterol are needed for membrane structure and function, making them essential for the development of functional cell walls. Therefore exposure to these fungicides results in abnormal growth and eventually death of sensitive fungi. Squalene-epoxidase inhibitor fungicides include thiocarbamates and allylaminess. The thiocarbamates include pyributicarb. The allylamines include naftifine and terbinafine.

(19) “Polyoxin fungicides” (Fungicide Resistance Action Committee (FRAC) code 19) inhibit chitin synthase. Examples include polyoxin.

(20) “Phenylurea fungicides” (Fungicide Resistance Action Committee (FRAC) code 20) are proposed to affect cell division. Examples include pencycuron.

(21) “Quinone inside inhibitor (QiI) fungicides” (Fungicide Resistance Action Committee (FRAC) code 21) inhibit Complex III mitochondrial respiration in fungi by affecting ubiquinol reductase. Reduction of ubiquinol is blocked at the “quinone inside” (Qᵢ) site of the cytochrome bc₁ complex, which is located in the inner mitochondrial membrane of fungi. Inhibiting mitochondrial respiration prevents normal fungal growth and development. Quinone inside inhibitor fungicides include cyanoimidazoles and sulfamoyltriazoles. The cyanoimidazoles include cyazofamid. The sulfamoyltriazoles include amisulbrom.

(22) “Benzamide fungicides” (Fungicide Resistance Action Committee (FRAC) code 22) inhibit mitosis by binding to β-tubulin and disrupting microtubule assembly Inhibition of microtubule assembly can disrupt cell division, transport within the cell and cell structure. Examples include zoxamide.

(23) “Enopyranuronic acid antibiotic fungicides” (Fungicide Resistance Action Committee (FRAC) code 23) inhibit growth of fungi by affecting protein biosynthesis. Examples include blasticidin-S.

(24) “Hexopyranosyl antibiotic fungicides” (Fungicide Resistance Action Committee (FRAC) code 24) inhibit growth of fungi by affecting protein biosynthesis. Examples include kasugamycin.

(25) “Glucopyranosyl antibiotic: protein synthesis fungicides” (Fungicide Resistance Action Committee (FRAC) code 25) inhibit growth of fungi by affecting protein biosynthesis. Examples include streptomycin.

(26) “Glucopyranosyl antibiotic: trehalase and inositol biosynthesis fungicides” (Fungicide Resistance Action Committee (FRAC) code 26) inhibit trehalase in inositol biosynthesis pathway. Examples include validamycin.

(27) “Cyanoacetamideoxime fungicides (Fungicide Resistance Action Committee (FRAC) code 27) include cymoxanil.

(28) “Carbamate fungicides” (Fungicide Resistance Action Committee (FRAC) code 28) are considered multi-site inhibitors of fungal growth. They are proposed to interfere with the synthesis of fatty acids in cell membranes, which then disrupts cell membrane permeability. Propamacarb, propamacarb-hydrochloride, iodocarb, and prothiocarb are examples of this fungicide class.

(29) “Oxidative phosphorylation uncoupling fungicides” (Fungicide Resistance Action Committee (FRAC) code 29) inhibit fungal respiration by uncoupling oxidative phosphorylation. Inhibiting respiration prevents normal fungal growth and development. This class includes 2,6-dinitroanilines such as fluazinam, pyrimidonehydrazones such as ferimzone and dinitrophenyl crotonates such as dinocap, meptyldinocap and binapacryl.

(30) “Organo tin fungicides” (Fungicide Resistance Action Committee (FRAC) code 30) inhibit adenosine triphosphate (ATP) synthase in oxidative phosphorylation pathway. Examples include fentin acetate, fentin chloride and fentin hydroxide.

(31) “Carboxylic acid fungicides” (Fungicide Resistance Action Committee (FRAC) code 31) inhibit growth of fungi by affecting deoxyribonucleic acid (DNA) topoisomerase type II (gyrase). Examples include oxolinic acid.

(32) “Heteroaromatic fungicides” (Fungicide Resistance Action Committee (FRAC) code 32) are proposed to affect DNA/ribonucleic acid (RNA) synthesis. Heteroaromatic fungicides include isoxazoles and isothiazolones. The isoxazoles include hymexazole and the isothiazolones include octhilinone.

(33) “Phosphonate fungicides” (Fungicide Resistance Action Committee (FRAC) code 33) include phosphorous acid and its various salts, including fosetyl-aluminum.

(34) “Phthalamic acid fungicides” (Fungicide Resistance Action Committee (FRAC) code 34) include teclofthalam.

(35) “Benzotriazine fungicides” (Fungicide Resistance Action Committee (FRAC) code 35) include triazoxide.

(36) “Benzene-sulfonamide fungicides” (Fungicide Resistance Action Committee (FRAC) code 36) include flusulfamide.

(37) “Pyridazinone fungicides” (Fungicide Resistance Action Committee (FRAC) code 37) include diclomezine.

(38) “Thiophene-carboxamide fungicides” (Fungicide Resistance Action Committee (FRAC) code 38) are proposed to affect ATP production. Examples include silthiofam.

(39) “Pyrimidinamide fungicides” (Fungicide Resistance Action Committee (FRAC) code 39) inhibit growth of fungi by affecting phospholipid biosynthesis and include diflumetorim.

(40) “Carboxylic acid amide (CAA) fungicides” (Fungicide Resistance Action Committee (FRAC) code 40) are proposed to inhibit phospholipid biosynthesis and cell wall deposition. Inhibition of these processes prevents growth and leads to death of the target fungus. Carboxylic acid amide fungicides include cinnamic acid amides, valinamide carbamates, carbamates and mandelic acid amides. The cinnamic acid amides include dimethomorph and flumorph. The valinamide carbamates include benthiavalicarb, benthiavalicarb-isopropyl, iprovalicarb, valifenalate and valiphenal. The carbamates include tolprocarb. The mandelic acid amides include mandipropamid, N-[2-[4-[[3-(4-chlorophenyl)-2-propyn-1-yl]oxy]-3-methoxyphenyl]ethyl]-3-methyl-2-[(methylsulfonyl)-amino]butanamide and N-[2-[4-[[3-(4-chlorophenyl)-2-propyn-1-yl]oxy]-3-methoxyphenyl]-ethyl]-3-methyl-2-[(ethylsulfonyl)amino]butanamide.

(41) “Tetracycline antibiotic fungicides” (Fungicide Resistance Action Committee (FRAC) code 41) inhibit growth of fungi by affecting complex 1 nicotinamide adenine dinucleotide (NADH) oxidoreductase. Examples include oxytetracycline.

(42) “Thiocarbamate fungicides” (Fungicide Resistance Action Committee (FRAC) code 42) include methasulfocarb.

(43) “Benzamide fungicides” (Fungicide Resistance Action Committee (FRAC) code 43) inhibit growth of fungi by delocalization of spectrin-like proteins. Examples include acylpicolide fungicides such as fluopicolide and fluopyram.

(44) “Host plant defense induction fungicides” (Fungicide Resistance Action Committee (FRAC) code P) induce host plant defense mechanisms. Host plant defense induction fungicides include benzo-thiadiazoles, benzisothiazoles and thiadiazole-carboxamides. The benzo-thiadiazoles include acibenzolar-S-methyl. The benzisothiazoles include probenazole. The thiadiazole-carboxamides include tiadinil and isotianil.

(45) “Multi-site contact fungicides” inhibit fungal growth through multiple sites of action and have contact/preventive activity. This class of fungicides includes: (45.1) “copper fungicides” (Fungicide Resistance Action Committee (FRAC) code M1)”, (45.2) “sulfur fungicides” (Fungicide Resistance Action Committee (FRAC) code M2), (45.3) “dithiocarbamate fungicides” (Fungicide Resistance Action Committee (FRAC) code M3), (45.4) “phthalimide fungicides” (Fungicide Resistance Action Committee (FRAC) code M4), (45.5) “chloronitrile fungicides” (Fungicide Resistance Action Committee (FRAC) code M5), (45.6) “sulfamide fungicides” (Fungicide Resistance Action Committee (FRAC) code M6), (45.7) “guanidine fungicides” (Fungicide Resistance Action Committee (FRAC) code M7), (45.8) “triazine fungicides” (Fungicide Resistance Action Committee (FRAC) code M8) and (45.9) “quinone fungicides” (Fungicide Resistance Action Committee (FRAC) code M9). “Copper fungicides” are inorganic compounds containing copper, typically in the copper(II) oxidation state; examples include copper oxychloride, copper sulfate and copper hydroxide, including compositions such as Bordeaux mixture (tribasic copper sulfate). “Sulfur fungicides” are inorganic chemicals containing rings or chains of sulfur atoms; examples include elemental sulfur. “Dithiocarbamate fungicides” contain a dithiocarbamate molecular moiety; examples include mancozeb, metiram, propineb, ferbam, maneb, thiram, zineb and ziram. “Phthalimide fungicides” contain a phthalimide molecular moiety; examples include folpet, captan and captafol. “Chloronitrile fungicides” contain an aromatic ring substituted with chloro and cyano; examples include chlorothalonil. “Sulfamide fungicides” include dichlofluanid and tolyfluanid. “Guanidine fungicides” include dodine, guazatine, iminoctadine albesilate and iminoctadine triacetate. “Triazine fungicides” include anilazine. “Quinone fungicides” include dithianon.

(46) “Fungicides other than fungicides of classes (1) through (45)” include certain fungicides whose mode of action may be unknown. These include: (46.1) “thiazole carboxamide fungicides” (Fungicide Resistance Action Committee (FRAC) code U5), (46.2) “phenyl-acetamide fungicides” (Fungicide Resistance Action Committee (FRAC) code U6), (46.3) “quinazolinone fungicides” (Fungicide Resistance Action Committee (FRAC) code U7), (46.4) “benzophenone fungicides” (Fungicide Resistance Action Committee (FRAC) code U8) and (46.5) “triazolopyrimidine fungicides”. The thiazole carboxamides include ethaboxam. The phenyl-acetamides include cyflufenamid and N-[[(cyclopropylmethoxy)-amino][6-(difluoromethoxy)-2,3-difluorophenyl]-methylene]benzeneacetamide. The quinazolinones include proquinazid. The benzophenones include metrafenone. The triazolopyrimidines include ametoctradin. Class (46) (i.e. “Fungicides other than classes (1) through (45)”) also includes bethoxazin, fluxapyroxad, neo-asozin (ferric methanearsonate), pyriofenone, pyrrolnitrin, quinomethionate, tebufloquin, N-[2-[4-[[3-(4-chlorophenyl)-2-propyn-1-yl]oxy]-3-methoxyphenyl]ethyl]-3-methyl-2-[(methylsulfonyl)amino]butanamide, N-[2-[4-[[3-(4-chlorophenyl)-2-propyn-1-yl]oxy]-3-methoxyphenyl]ethyl]-3-methyl-2-[(ethylsulfonyl)amino]butanamide, 2-[[2-fluoro-5-(trifluoromethyl)phenyl]thio]-2-[3-(2-methoxyphenyl)-2-thiazolidinylidene]acetonitrile, 3-[5-(4-chlorophenyl)-2,3-dimethyl-3-isoxazolidinyl]pyridine, 4-fluorophenyl N-[1-[[[1-(4-cyanophenyl)ethyl]sulfonyl]methyl]-propyl]carbamate, 5-chloro-6-(2,4,6-trifluorophenyl)-7-(4-methylpiperidin-1-yl) [1,2,4]-triazolo[1,5-a]pyrimidine, N-(4-chloro-2-nitrophenyl)-N-ethyl-4-methylbenz enesulfonamide, N-[[(cyclopropylmethoxy)amino][6-(difluoromethoxy)-2,3-difluorophenyl]methylene]-benzeneacetamide, N-[4-[4-chloro-3-(trifluoromethyl)phenoxy]-2,5-dimethylphenyl]-N-ethyl-N-methylmethanimidamide, 1-[(2-propenylthio)carbonyl]-2-(1-methylethyl)-4-(2-methylphenyl)-5-amino-1H-pyrazol-3-one, N-[4-[[3-[(4-chlorophenyl)methyl]-1,2,4-thia-diazol-5-yl]oxy]-2,5-dimethylphenyl]-N-ethyl-N-methyl-methanimidamide, 1,1-dimethyl-ethyl N-[6-[[[[1-methyl-1H-tetrazol-5-yl)phenylmethylene]amino]oxy]methyl-2-pyridinyl]-carbamate, 3-butyn-1-yl N-[6-[[[[1-methyl-1H-tetrazol-5-yl)phenylmethylene]amino]oxy]-methyl]-2-pyridinyl]carbamate, 2,6-dimethyl-1H,5H-[1,4]dithiino[2,3-c:5,6-c']0 dipyrrole-1,3,5,7(2H,6H)-tetrone, 5-fluoro-2-[(4-methylphenyl)methoxy]-4-pyrimidinamine and 5-fluoro-2-[(4-fluorophenyl)methoxy]-4-pyrimidinamine.

Therefore of note is a mixture (i.e. composition) comprising a compound of Formula 1 and at least one fungicidal compound selected from the group consisting of the aforedescribed classes (1) through (46). Also of note is a composition comprising said mixture (in fungicidally effective amount) and further comprising at least one additional component selected from the group consisting of surfactants, solid diluents and liquid diluents. Of particular note is a mixture (i.e. composition) comprising a compound of Formula 1 and at least one fungicidal compound selected from the group of specific compounds listed above in connection with classes (1) through (46). Also of particular note is a composition comprising said mixture (in fungicidally effective amount) and further comprising at least one additional surfactant selected from the group consisting of surfactants, solid diluents and liquid diluents.

Examples of other biologically active compounds or agents with which compounds of this invention can be formulated are: insecticides such as abamectin, acephate, acetamiprid, acrinathrin, amidoflumet (S-1955), avermectin, azadirachtin, azinphos-methyl, bifenthrin, bifenazate, buprofezin, carbofuran, cartap, chlorantraniliprole, chlorfenapyr, chlorfluazuron, chlorpyrifos, chlorpyrifos-methyl, chromafenozide, clothianidin, cyantraniliprole (3-bromo-1-(3-chloro-2-pyridinyl)-N4-[4-cyano-2-methyl-6-[(methylamino)carbonyl]phenyl]-1H-pyrazole-5-carboxamide), cyflumetofen, cyfluthrin, beta-cyfluthrin, cyhalothrin, lambda-cyhalothrin, cypermethrin, cyromazine, deltamethrin, diafenthiuron, diazinon, dieldrin, diflubenzuron, dimefluthrin, dimethoate, dinotefuran, diofenolan, emamectin, endosulfan, esfenvalerate, ethiprole, fenothiocarb, fenoxycarb, fenpropathrin, fenvalerate, fipronil, flonicamid, flubendiamide, flucythrinate, tau-fluvalinate, flufenerim (UR-50701), flufenoxuron, fonophos, halofenozide, hexaflumuron, hydramethylnon, imidacloprid, indoxacarb, isofenphos, lufenuron, malathion, meperfluthrin, metaflumizone, metaldehyde, methamidophos, methidathion, methomyl, methoprene, methoxychlor, methoxyfenozide, metofluthrin, milbemycin oxime, monocrotophos, nicotine, nitenpyram, nithiazine, novaluron, noviflumuron (XDE-007), oxamyl, parathion, parathion-methyl, permethrin, phorate, phosalone, phosmet, phosphamidon, pirimicarb, profenofos, profluthrin, pymetrozine, pyrafluprole, pyrethrin, pyridalyl, pyrifluquinazon, pyriprole, pyriproxyfen, rotenone, ryanodine, spinetoram, spinosad, spirodiclofen, spiromesifen (BSN 2060), spirotetramat, sulfoxaflor, sulprofos, tebufenozide, teflubenzuron, tefluthrin, terbufos, tetrachlorvinphos, tetramethylfluthrin, thiacloprid, thiamethoxam, thiodicarb, thiosultap-sodium, tolfenpyrad, tralomethrin, triazamate, trichlorfon and triflumuron; and biological agents including entomopathogenic bacteria, such as Bacillus thuringiensis subsp. aizawai, Bacillus thuringiensis subsp. kurstaki, and the encapsulated delta-endotoxins of Bacillus thuringiensis (e.g., Cellcap, MPV, MPVII); entomopathogenic fungi, such as green muscardine fungus; and entomopathogenic virus including baculovirus, nucleopolyhedro virus (NPV) such as HzNPV, AfNPV; and granulosis virus (GV) such as CpGV.

Compounds of this invention and compositions thereof can be applied to plants genetically transformed to express proteins toxic to invertebrate pests (such as Bacillus thuringiensis delta-endotoxins). The effect of the exogenously applied fungicidal compounds of this invention may be synergistic with the expressed toxin proteins.

General references for agricultural protectants (i.e. insecticides, fungicides, nematocides, acaricides, herbicides and biological agents) include The Pesticide Manual, 13th Edition, C. D. S. Tomlin, Ed., British Crop Protection Council, Farnham, Surrey, U.K., 2003 and The BioPesticide Manual, 2nd Edition, L. G. Copping, Ed., British Crop Protection Council, Farnham, Surrey, U.K., 2001.

For embodiments where one or more of these various mixing partners are used, the weight ratio of these various mixing partners (in total) to the compound of Formula 1 is typically between about 1:3000 and about 3000:1. Of note are weight ratios between about 1:300 and about 300:1 (for example ratios between about 1:30 and about 30:1). One skilled in the art can easily determine through simple experimentation the biologically effective amounts of active ingredients necessary for the desired spectrum of biological activity. It will be evident that including these additional components may expand the spectrum of diseases controlled beyond the spectrum controlled by the compound of Formula 1 alone.

In certain instances, combinations of a compound of this invention with other biologically active (particularly fungicidal) compounds or agents (i.e. active ingredients) can result in a greater-than-additive (i.e. synergistic) effect. Reducing the quantity of active ingredients released in the environment while ensuring effective pest control is always desirable. When synergism of fungicidal active ingredients occurs at application rates giving agronomically satisfactory levels of fungal control, such combinations can be advantageous for reducing crop production cost and decreasing environmental load.

Of note is a combination of a compound of Formula 1 with at least one other fungicidal active ingredient. Of particular note is such a combination where the other fungicidal active ingredient has different site of action from the compound of Formula 1. In certain instances, a combination with at least one other fungicidal active ingredient having a similar spectrum of control but a different site of action will be particularly advantageous for resistance management. Thus, a composition of the present invention can further comprise a biologically effective amount of at least one additional fungicidal active ingredient having a similar spectrum of control but a different site of action.

Of particular note are compositions which in addition to compound of Formula 1 include at least one compound selected from the group consisting of (1) alkylenebis(dithiocarbamate) fungicides; (2) cymoxanil; (3) phenylamide fungicides; (4) proquinazid (6-iodo-3-propyl-2-propyloxy-4(3H)-quinazolinone); (5) chlorothalonil; (6) carboxamides acting at complex II of the fungal mitochondrial respiratory electron transfer site; (7) quinoxyfen; (8) metrafenone; (9) cyflufenamid; (10) cyprodinil; (11) copper compounds; (12) phthalimide fungicides; (13) fosetyl-aluminum; (14) benzimidazole fungicides; (15) cyazofamid; (16) fluazinam; (17) iprovalicarb; (18) propamocarb; (19) validomycin; (20) dichlorophenyl dicarboximide fungicides; (21) zoxamide; (22) fluopicolide; (23) mandipropamid; (24) carboxylic acid amides acting on phospholipid biosynthesis and cell wall deposition; (25) dimethomorph; (26) non-DMI sterol biosynthesis inhibitors; (27) inhibitors of demethylase in sterol biosynthesis; (28) bc₁ complex fungicides; and salts of compounds of (1) through (28).

Further descriptions of classes of fungicidal compounds are provided below.

Sterol biosynthesis inhibitors (group (27)) control fungi by inhibiting enzymes in the sterol biosynthesis pathway. Demethylase-inhibiting fungicides have a common site of action within the fungal sterol biosynthesis pathway, involving inhibition of demethylation at position 14 of lanosterol or 24-methylene dihydrolanosterol, which are precursors to sterols in fungi. Compounds acting at this site are often referred to as demethylase inhibitors, DMI fungicides, or DMIs. The demethylase enzyme is sometimes referred to by other names in the biochemical literature, including cytochrome P-450 (14DM). The demethylase enzyme is described in, for example, J. Biol. Chem. 1992, 267, 13175-79 and references cited therein. DMI fungicides are divided between several chemical classes: azoles (including triazoles and imidazoles), pyrimidines, piperazines and pyridines. The triazoles include azaconazole, bromuconazole, cyproconazole, difenoconazole, diniconazole (including diniconazole-M), epoxiconazole, etaconazole, fenbuconazole, fluquinconazole, flusilazole, flutriafol, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole and uniconazole. The imidazoles include clotrimazole, econazole, imazalil, isoconazole, miconazole, oxpoconazole, prochloraz and triflumizole. The pyrimidines include fenarimol, nuarimol and triarimol. The piperazines include triforine. The pyridines include buthiobate and pyrifenox. Biochemical investigations have shown that all of the above mentioned fungicides are DMI fungicides as described by K. H. Kuck et al. in Modern Selective Fungicides—Properties, Applications and Mechanisms of Action, H. Lyr (Ed.), Gustav Fischer Verlag: New York, 1995, 205-258.

bc₁ Complex Fungicides (group 28) have a fungicidal mode of action which inhibits the bc₁ complex in the mitochondrial respiration chain. The bc₁ complex is sometimes referred to by other names in the biochemical literature, including complex III of the electron transfer chain, and ubihydroquinone:cytochrome c oxidoreductase. This complex is uniquely identified by Enzyme Commission number EC1.10.2.2. The bc₁ complex is described in, for example, J. Biol. Chem. 1989, 264, 14543-48; Methods Enzymol. 1986, 126, 253-71; and references cited therein. Strobilurin fungicides such as azoxystrobin, dimoxystrobin, enestroburin (SYP-Z071), fluoxastrobin, kresoxim-methyl, metominostrobin, orysastrobin, picoxystrobin, pyraclostrobin, pyrametostrobin, pyraoxystrobin and trifloxystrobin are known to have this mode of action (H. Sauter et al., Angew. Chem. Int. Ed. 1999, 38, 1328-1349). Other fungicidal compounds that inhibit the bc₁ complex in the mitochondrial respiration chain include famoxadone and fenamidone.

Alkylenebis(dithiocarbamate)s (group (1)) include compounds such as mancozeb, maneb, propineb and zineb. Phenylamides (group (3)) include compounds such as metalaxyl, benalaxyl, furalaxyl and oxadixyl. Carboxamides (group (6)) include compounds such as boscalid, carboxin, fenfuram, flutolanil, furametpyr, mepronil, oxycarboxin, thifluzamide, penthiopyrad and N-[2-(1,3-dimethylbutyl)phenyl]-5-fluoro-1,3-dimethyl-1H-pyrazole-4-carboxamide (PCT Patent Publication WO 2003/010149), and are known to inhibit mitochondrial function by disrupting complex II (succinate dehydrogenase) in the respiratory electron transport chain. Copper compounds (group (11)) include compounds such as copper oxychloride, copper sulfate and copper hydroxide, including compositions such as Bordeaux mixture (tribasic copper sulfate). Phthalimides (group (12)) include compounds such as folpet and captan. Benzimidazole fungicides (group (14)) include benomyl and carbendazim. Dichlorophenyl dicarboximide fungicides (group (20)) include chlozolinate, dichlozoline, iprodione, isovaledione, myclozolin, procymidone and vinclozolin.

Non-DMI sterol biosynthesis inhibitors (group (26)) include morpholine and piperidine fungicides. The morpholines and piperidines are sterol biosynthesis inhibitors that have been shown to inhibit steps in the sterol biosynthesis pathway at a point later than the inhibitions achieved by the DMI sterol biosynthesis (group (27)). The morpholines include aldimorph, dodemorph, fenpropimorph, tridemorph and trimorphamide. The piperidines include fenpropidin.

Of further note are combinations of compounds of Formula 1 with azoxystrobin, kresoxim-methyl, trifloxystrobin, pyraclostrobin, picoxystrobin, dimoxystrobin, metominostrobin/fenominostrobin, carbendazim, chlorothalonil, quinoxyfen, metrafenone, cyflufenamid, fenpropidine, fenpropimorph, bromuconazole, cyproconazole, difenoconazole, epoxiconazole, fenbuconazole, flusilazole, hexaconazole, ipconazole, metconazole, penconazole, propiconazole, proquinazid, prothioconazole, tebuconazole, triticonazole, famoxadone, prochloraz, penthiopyrad and boscalid (nicobifen).

Specifically preferred mixtures (compound numbers refer to compounds in Index Table A) are selected from the group: combinations of Compound 5, Compound 6, or Compound 8 with azoxystrobin, combinations of Compound 5, Compound 6 or Compound 8 with kresoxim-methyl, combinations of Compound 5, Compound 6 or Compound 8 with trifloxystrobin, combinations of Compound 5, Compound 6 or Compound 8 with picoxystrobin, combinations of Compound 5, Compound 6 or Compound 8 with quinoxyfen, combinations of Compound 5, Compound 6 or Compound 8 with metrafenone, combinations of Compound 5, Compound 6 or Compound 8 with fenpropidine, combinations of Compound 5, Compound 6 or Compound 8 with fenpropimorph, combinations of Compound 5, Compound 6 or Compound 8 with cyproconazole, combinations of Compound 5, Compound 6 or Compound 8 with epoxiconazole, combinations of Compound 5, Compound 6 or Compound 8 with flusilazole, combinations of Compound 5, Compound 6 or Compound 8 with metconazole, combinations of Compound 5, Compound 6 or Compound 8 with propiconazole, combinations of Compound 5, Compound 6 or Compound 8 with proquinazid, combinations of Compound 5, Compound 6 or Compound 8 with prothioconazole, combinations of Compound 5, Compound 6 or Compound 8 with tebuconazole, combinations of Compound 5, Compound 6 or Compound 8 with triticonazole, combinations of Compound 5, Compound 6 or Compound 8 with famoxadone, combinations of Compound 5, Compound 6 or Compound 8 with penthiopyrad, combinations of Compound 5, Compound 6 or Compound 8 with 3-(difluoromethyl)-1-methyl-N-(3′,4′,5′-trifluoro[1,1′-biphenyl]-2-yl)-1H-pyrazole-4-carboxamide, combinations of Compound 5, Compound 6 or Compound 8 with 5-ethyl-6-octyl-[1,2,4]triazole[1,5-a]pyrimidin-7-amine, and Compound 5, Compound 6, or Compound 8 with Initium®.

The control efficacy of compounds of this invention on specific pathogens is demonstrated in TABLE A below. The pathogen control protection afforded by the compounds is not limited, however, to the test results in TABLE A. Descriptions of the compounds are provided in Index Table A below. The following abbreviations are used in the index table: Me is methyl, “Cmpd. No.” means compound number, and “Ex.” stands for “Example” and is followed by a number indicating in which example the compound is prepared. In Index Table A the numerical value reported in the column “AP⁺ (M+1)”, is the molecular weight of the observed molecular ion formed by addition of H⁺ (molecular weight of 1) to the molecule having the greatest isotopic abundance (i.e. M). The presence of molecular ions containing one or higher atomic weight isotopes of lower abundance (e.g., ³⁷Cl, ⁸¹Br) is not reported. The reported M+1 peaks were observed by mass spectrometry using atmospheric pressure chemical ionization (AP⁺).

INDEX TABLE A

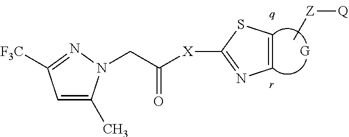

| Cmpd. No. | X | G | Z | Q | AP⁺ (M + 1) |
|---|---|---|---|---|---|
| 1 (Note 1) | X² | G-31 | NHCH(Me) | Ph | 529 |
| 2 (Note 1) | X² | G-31 | N(Me)CH(Me) | Ph | 543 |
| 3 (Note 1) | X² | G-31 | N(Me) | 1,2,3,4-tetrahydro-1- | 569 |
| | | | | naphthalenyl | |
| 4 | X¹ | G-32 | NOCH₂ | Ph | 532 |
| 5 (Ex. 2) | X¹ | G-13 | CH₂ | 2,6-di-F—Ph | 553 |
| 6 (Ex. 3) | X¹ | G-14 | CH | 2,6-di-F—Ph | 551 |
| 7 | X¹ | G-33 | CH₂ | 2,6-di-F—Ph | 568 |
| 8 (Ex. 1) | X¹ | G-15 | CH₂ | Ph | 518 |
| 9 | X¹ | G-34 | CH₂ | 2,6-di-F—Ph | 540 |

Note 1: (R)-enantiomer.

### Biological Examples of the Invention

General protocol for preparing test suspensions for Tests A-B2: The test compounds were first dissolved in acetone in an amount equal to 3% of the final volume and then suspended at the desired concentration (in ppm) in acetone and purified water (50/50 mix by volume) containing 250 ppm of the surfactant Trem® 014 (polyhydric alcohol esters). The resulting test suspensions were then used in Tests A-B2. Spraying a 40 ppm test suspension to the point of run-off on the test plants was equivalent to a rate of 160 g/ha.

### Test A

Grape seedlings were inoculated with a spore suspension of Plasmopara viticola (the causal agent of grape downy mildew) and incubated in a saturated atmosphere at 20° C. for 24 h. After a short drying period, the grape seedlings were sprayed with the test suspension to the point of run-off, then moved to a growth chamber at 20° C. for 5 days, and then back into a saturated atmosphere at 20° C. for 24 h. Upon removal, visual disease ratings were made.

### Test B1

The test suspension was sprayed to the point of run-off on tomato seedlings. The following day the seedlings were inoculated with a spore suspension of Phytophthora infestans (the causal agent of tomato late blight) and incubated in a saturated atmosphere at 20° C. for 24 h, and then moved to a growth chamber at 20° C. for 5 days, after which time visual disease ratings were made.

### Test B2

Tomato seedlings were inoculated with a spore suspension of Phytophthora infestans (the causal agent of tomato late blight) and incubated in a saturated atmosphere at 20° C. for 17 h. After a short drying period, the tomato seedlings were sprayed with test suspension to the point of run-off, and then moved to a growth chamber at 20° C. for 4 days, after which time visual disease ratings were made.

Results for Tests A-B2 are given in Table A. In the Table, a rating of 100 indicates 100% disease control and a rating of 0 indicates no disease control (relative to the controls). All results are for 40 ppm.

TABLE A

| Cmpd. No. | Test A | Test B1 | Test B2 |
|---|---|---|---|
| 1 | 0 | 0 | 0 |
| 2 | 0 | 17 | 0 |
| 3 | 0 | 40 | 9 |
| 4 | 0 | 66 | 0 |
| 5 | 99 | 100 | 99 |
| 6 | 78 | 100 | 78 |
| 7 | 96 | 77 | 35 |
| 8 | 100 | 100 | 99 |
| 9 | 29 | 31 | 0 |

What is claimed is:

1. A compound selected from Formula 1, tautomers, N-oxides, and salts thereof, 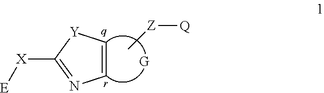
wherein
E is a radical selected from the group consisting of 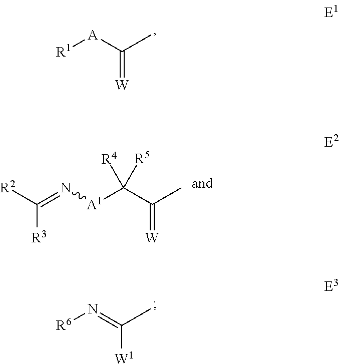
X is a radical selected from the group consisting of 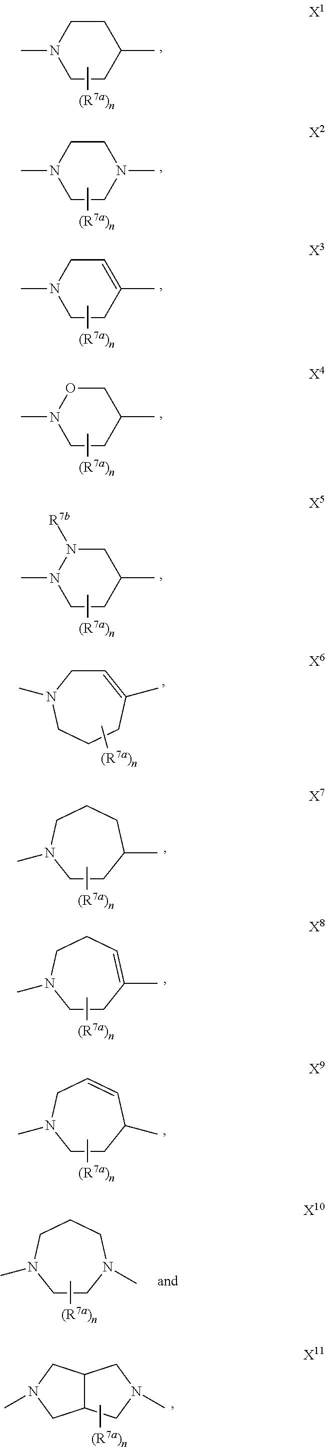
wherein the bond projecting to the left is connected to E, and the bond projecting to the right is connected to the carbon atom in Formula 1;
Y is O, S, NH or N(CH₃);
G together with the two carbon atoms identified as “q” and “r” in Formula 1 forms a 5- to 6-membered ring containing ring members selected from carbon atoms and up to 2 heteroatoms independently selected from up to 1 O, up to 1 S and up to 2 N atoms, wherein up to 1 carbon atom ring member is selected from C(═O), C(═S) and C(═NOH), the ring optionally substituted with up to 2 substituents independently selected from R⁸ on carbon atom ring members and methyl on nitrogen atom ring members;
Z is a saturated, partially unsaturated or fully unsaturated chain containing 1- to 3-atoms selected from up to 3 carbon, up to 1 O, up to 1 S and up to 2 N atoms, the chain optionally substituted with up to 2 substituents independently selected from R^{9a} on carbon atoms and R^{9b} on nitrogen atoms;
Q is phenyl or naphthalenyl, each optionally substituted with up to 3 substituents independently selected from R^{10a}; or
a 5- to 6-membered heteroaromatic ring or an 8- to 11-membered heteroaromatic bicyclic ring system, each ring or ring system containing ring members selected from carbon atoms and up to 4 heteroatoms independently selected from up to 2 O, up to 2 S and up to 4 N atoms, each ring or ring system optionally substituted with up to 3 substituents independently selected from R^{10a} on carbon atom ring members and R^{10b} on nitrogen atom ring members; or
a 3- to 7-membered nonaromatic carbocyclic ring, a 5- to 7-membered nonaromatic heterocyclic ring or an 8- to 11-membered nonaromatic bicyclic ring system, each ring or ring system containing ring members selected from carbon atoms and up to 4 heteroatoms independently selected from up to 2 O, up to 2 S and up to 4 N atoms, wherein up to 3 carbon atom ring members are independently selected from C(═O) and C(═S), and the sulfur atom ring members are independently selected from S(═O)ₛ(═NR²⁰)_{f}, each ring or ring system optionally substituted with up to 3 substituents independently selected from R^{10a} on carbon atom ring members and R^{10b} on nitrogen atom ring members;
A is CH(R¹¹), N(R¹²) or C(═O);
A¹ is O, S, C(R¹⁴)₂; N(R¹³); —OC(R¹⁴)₂—, —SC(R¹⁴)₂—) or —N(R¹³)C(R¹⁴)₂—, wherein the bond projecting to the left is connected to the nitrogen atom, and the bond projecting to the right is connected to the carbon atom in Formula 1;
W is O or S;
W¹ is OR¹⁵; SR¹⁶, NR¹⁷R¹⁸ or R¹⁹;
R¹ and R⁶ are each optionally substituted phenyl, optionally substituted naphthalenyl or an optionally substituted 5- to 6-membered heteroaromatic ring; or cyano, C₁-C₈ alkyl, C₁-C₈ haloalkyl, C₂-C₈ alkenyl, C₂-C₈ haloalkenyl, C₂-C₈ alkynyl, C₂-C₈ haloalkynyl, C₃-C₈ cycloalkyl, C₃-C₈ halocycloalkyl, C₄-C₁₀ alkylcycloalkyl, C₄-C₁₀ cycloalkylalkyl, C₄-C₁₀ halocycloalkylalkyl, C₅-C₁₀ alkylcycloalkylalkyl, C₂-C₈ alkoxyalkyl, C₂-C₈ haloalkoxyalkyl, C₄-C₁₀ cycloalkoxyalkyl, C₃-C₁₀ alkoxyalkoxyalkyl, C₂-C₈ alkylthioalkyl, C₂-C₈ haloalkylthioalkyl, C₂-C₈ alkylsulfinylalkyl, C₂-C₈ alkylsulfonylalkyl, C₂-C₈ alkylaminoalkyl, C₂-C₈ haloalkylaminoalkyl, C₃-C₁₀ dialkylaminoalkyl, C₄-C₁₀ cycloalkylaminoalkyl, C₃-C₈ alkoxycarbonylalkyl, C₃-C₈ haloalkoxycarbonylalkyl, C₁-C₈ alkoxy, C₁-C₈ haloalkoxy, C₂-C₈ alkenyloxy, C₂-C₈ haloalkenyloxy, C₂-C₈ alkynyloxy, C₃-C₈ haloalkynyloxy, C₃-C₈ cycloalkoxy, C₃-C₈ halocycloalkoxy, C₄-C₁₀ cycloalkylalkoxy, C₂-C₈ alkoxyalkoxy, C₂-C₈ alkylcarbonyloxy, C₂-C₈ haloalkylcarbonyloxy, C₁-C₈ alkylthio, C₁-C₈ haloalkylthio, C₃-C₈ cycloalkylthio, C₁-C₈ alkylamino, C₁-C₈ haloalkylamino, C₂-C₈ dialkylamino, C₂-C₈ halodialkylamino, C₃-C₈ cycloalkylamino, C₁-C₈ alkylsulfonylamino, C₁-C₈ halo alkylsulfonylamino, C₂-C₈ alkylcarbonylamino, C₂-C₈ haloalkylcarbonylamino, C₃-C₁₀ trialkylsilyl, pyrrolidinyl, piperidinyl or morpholinyl;
R² is H, amino, cyano, halogen, —CH(═O), —C(═O)OH, —C(═O)NH₂, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₃-C₆ cycloalkyl, C₃-C₆ halocycloalkyl, C₃-C₆ cycloalkenyl, C₃-C₆ halocycloalkenyl, C₄-C₆ alkylcycloalkyl, C₄-C₆ cycloalkylalkyl, C₄-C₆ halocycloalkylalkyl, C₂-C₆ alkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl, C₂-C₆ alkylcarbonyl, C₂-C₆ haloalkylcarbonyl, C₄-C₆ cycloalkylcarbonyl, C₂-C₆ alkoxycarbonyl, C₄-C₆ cycloalkoxycarbonyl, C₅-C₆ cycloalkylalkoxycarbonyl, C₂-C₆ alkylaminocarbonyl, C₃-C₆ dialkylaminocarbonyl, C₁-C₆ alkoxy, C₁-C₆ haloalkoxy, C₂-C₆ alkenyloxy, C₂-C₆ haloalkenyloxy, C₂-C₆ alkynyloxy, C₃-C₆ haloalkynyloxy, C₃-C₆ cycloalkoxy, C₃-C₆ halocycloalkoxy, C₂-C₆ alkoxyalkoxy, C₂-C₆ alkylcarbonyloxy, C₂-C₆ haloalkylcarbonyloxy, C₁-C₆ alkylthio, C₁-C₆ haloalkylthio, C₃-C₆ cycloalkylthio, C₁-C₆ alkylamino, C₂-C₆ dialkylamino, C₁-C₆ haloalkylamino, C₂-C₆ halodialkylamino, C₃-C₆ cycloalkylamino, C₁-C₆ alkylsulfonylamino, C₁-C₆ halo alkylsulfonylamino C₂-C₆ alkylcarbonylamino or C₂-C₆ haloalkylcarbonylamino;
R³ is H, cyano, halogen, hydroxy, C₁-C₃ alkyl, C₁-C₃ haloalkyl, C₁-C₃ alkoxy or C₁-C₃ haloalkoxy; or
R² and R³ are taken together with the carbon atom to which they are attached to form a 3- to 7-membered ring containing ring members selected from carbon atoms and up to 4 heteroatoms independently selected from up to 2 O, up to 2 S and up to 2 N atoms, wherein up to 3 carbon atom ring members are independently selected from C(═O) and C(═S), and the sulfur atom ring members are independently selected from S(═O)ₛ(═NR²⁰)_{f}, the ring optionally substituted with up to 4 substituents independently selected from halogen, cyano, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
R⁴ is optionally substituted phenyl, optionally substituted naphthalenyl or an optionally substituted 5- to 6-membered heteroaromatic ring; or H, cyano, halogen, hydroxy, —CH(═O), C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₁-C₄ alkoxy, C₁-C₄ haloalkoxy, C₂-C₄ alkylcarbonyloxy, C₂-C₄ haloalkylcarbonyloxy, C₂-C₅ alkoxycarbonyloxy, C₂-C₅ alkylaminocarbonyloxy, C₃-C₅ dialkylaminocarbonyloxy, C₁-C₄ alkylthio, C₁-C₄ haloalkylthio, C₁-C₄ alkylsulfinyl, C₁-C₄ haloalkylsulfinyl, C₁-C₄ alkylsulfonyl, C₁-C₄ haloalkylsulfonyl C₂-C₄ alkylcarbonyl, C₂-C₄ haloalkylcarbonyl, C₂-C₅ alkoxycarbonyl, C₂-C₅ alkylaminocarbonyl or C₃-C₅ dialkylaminocarbonyl;
R⁵ is H, C₁-C₃ alkyl or C₁-C₃ haloalkyl;
each R^{7a} is independently halogen, cyano, hydroxy, C₁-C₄ alkyl, C₁-C₄ haloalkyl, C₂-C₄ alkenyl or C₁-C₄ alkoxy; or
two R^{7a} are taken together as C₁-C₄ alkylene or C₂-C₄ alkenylene to form a bridged or fused ring system;
R^{7b} is H, cyano, C₁-C₃ alkyl, C₁-C₃ haloalkyl, C₃-C₆ cycloalkyl, C₁-C₃ alkoxy, C₂-C₃ alkylcarbonyl or C₂-C₃ alkoxycarbonyl;
each R⁸ is independently cyano, halogen, hydroxy, methyl or methoxy;
each R^{9a} is independently cyano, halogen, hydroxy, C₁-C₄ alkyl, C₁-C₄ haloalkyl, C₃-C₆ cycloalkyl, C₂-C₄ alkoxyalkyl, C₁-C₄ alkoxy, C₁-C₄ haloalkoxy, C₂-C₄ alkylcarbonyl or C₂-C₄ alkoxycarbonyl;
each R^{9b} is independently cyano, C₁-C₄ alkyl, C₁-C₄ haloalkyl, C₃-C₆ cycloalkyl, C₁-C₄ alkoxy, C₂-C₄ alkylcarbonyl or C₂-C₄ alkoxycarbonyl;
each R^{10a} is independently amino, cyano, halogen, hydroxy, nitro, C₁-C₆ alkyl, C₁-C₆ haloalkyl, C₂-C₆ alkenyl, C₂-C₆ haloalkenyl, C₂-C₆ alkynyl, C₂-C₆ haloalkynyl, C₁-C₄ hydroxyalkyl, C₃-C₆ cycloalkyl, C₃-C₆ halocycloalkyl, C₄-C₁₀ cycloalkylalkyl, C₄-C₁₀ alkylcycloalkyl, C₅-C₁₀ alkylcycloalkylalkyl, C₆-C₁₄ cycloalkylcycloalkyl, C₂-C₄ alkoxyalkyl, C₁-C₄ alkoxy, C₁-C₄ haloalkoxy, C₂-C₆ alkylcarbonyloxy, C₁-C₄ alkylthio, C₁-C₄ haloalkylthio, C₂-C₆ alkylcarbonylthio, C₁-C₄ alkylsulfinyl, C₁-C₄ haloalkylsulfinyl, C₁-C₄ alkylsulfonyl, C₁-C₄ haloalkylsulfonyl, C₁-C₄ alkylamino, C₂-C₈ dialkylamino, C₃-C₆ cycloalkylamino, C₂-C₄ alkylcarbonyl, C₂-C₆ alkoxycarbonyl, C₂-C₆ alkylaminocarbonyl, C₃-C₈ dialkylaminocarbonyl or C₃-C₆ trialkylsilyl; or
phenyl or naphthalenyl, each optionally substituted with up to 3 substituents independently selected from cyano, halogen, C₁-C₂ alkyl, C₁-C₂ haloalkyl, C₁-C₂ alkoxy and C₁-C₂ haloalkoxy; or
a 5- to 6-membered heteroaromatic ring containing ring members selected from carbon atoms and up to 4 heteroatoms independently selected from up to 2 O, up to 2 S and up to 4 N atoms, the ring optionally substituted with up to 3 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members; or
a 3- to 7-membered nonaromatic ring containing ring members selected from carbon atoms and up to 4 heteroatoms independently selected from up to 2 O, up to 2 S and up to 4 N atoms, wherein up to 3 carbon atom ring members are independently selected from C(═O) and C(═S), the ring optionally substituted with up to 3 substituents independently selected from cyano, halogen, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
R^{10b} is cyano, C₁-C₃ alkyl, C₁-C₃ haloalkyl, C₃-C₆ cycloalkyl C₁-C₃ alkoxy, C₂-C₃ alkylcarbonyl or C₂-C₃ alkoxycarbonyl;
R¹¹ is H, cyano, halogen, hydroxy, —CH(═O), C₁-C₄ alkyl, C₁-C₄ haloalkyl, C₂-C₄ alkenyl, C₂-C₄ haloalkenyl, C₂-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₃-C₅ alkoxycarbonylalkyl, C₂-C₄ alkylcarbonyl, C₂-C₄ haloalkylcarbonyl, C₂-C₅ alkoxycarbonyl, C₂-C₅ alkylaminocarbonyl, C₃-C₅ dialkylaminocarbonyl, C₁-C₄ alkoxy, C₁-C₄ haloalkoxy, C₁-C₄ alkylthio, C₁-C₄ haloalkylthio, C₁-C₄ alkylsulfinyl, C₁-C₄ haloalkylsulfinyl, C₁-C₄ alkylsulfonyl or C₁-C₄ haloalkylsulfonyl;
R¹² is H, C₁-C₄ alkyl, C₁-C₄ haloalkyl, C₂-C₄ alkenyl, C₂-C₄ haloalkenyl, C₃-C₄ alkynyl, C₂-C₄ haloalkynyl, C₂-C₄ alkoxyalkyl, C₂-C₄ alkylthioalkyl, C₂-C₄ alkylsulfinylalkyl, C₂-C₄ alkylsulfonylalkyl, C₃-C₅ alkoxycarbonylalkyl, C₁-C₄ alkylsulfonyl, C₁-C₄ haloalkylsulfonyl, C₂-C₄ alkylcarbonyl, C₂-C₄ haloalkylcarbonyl, C₂-C₅ alkoxycarbonyl, C₂-C₅ alkylaminocarbonyl or C₃-C₅ dialkylaminocarbonyl;
R¹³ is H, cyano, C₁-C₄ alkyl, C₁-C₄ haloalkyl, C₂-C₄ alkoxyalkyl, C₂-C₄ alkylthioalkyl, C₁-C₄ alkylsulfonyl, C₁-C₄ haloalkylsulfonyl, C₂-C₄ alkylcarbonyl, C₂-C₄ haloalkylcarbonyl, C₂-C₄ alkoxycarbonyl, C₂-C₄ alkylaminocarbonyl or C₃-C₅ dialkylaminocarbonyl; or
R¹³ and R³ are taken together with the atoms to which they are attached to form a 5- to 7-membered partially saturated ring containing ring members selected from carbon atoms and up to 3 heteroatoms independently selected from up to 1 O, up to 1 S and up to 1 N atom, the ring optionally substituted with up to 3 substituents independently selected from cyano, halogen, nitro, C₁-C₂ alkyl, C₁-C₂ haloalkyl, C₁-C₂ alkoxy and C₁-C₂ haloalkoxy on carbon atom ring members and cyano, C₁-C₂ alkyl and C₁-C₂ alkoxy on nitrogen atom ring members;
each R¹⁴ is independently H, C₁-C₃ alkyl or C₁-C₃ haloalkyl;
R¹⁵ and R¹⁶ are each C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₃-C₆ halocycloalkyl, C₄-C₈ alkylcycloalkyl, C₄-C₈ cycloalkylalkyl, C₄-C₈ halocycloalkylalkyl, C₅-C₈ alkylcycloalkylalkyl, C₂-C₆ alkoxyalkyl, C₄-C₈ cycloalkoxyalkyl, C₃-C₆ alkoxyalkoxyalkyl, C₂-C₆ alkylthioalkyl, C₂-C₆ alkylsulfinylalkyl, C₂-C₆ alkylsulfonylalkyl, C₂-C₆ alkylaminoalkyl, C₂-C₆ haloalkylaminoalkyl, C₃-C₆ dialkylaminoalkyl, C₄-C₈ cycloalkylaminoalkyl, C₂-C₆ alkylcarbonyl, C₂-C₆ haloalkylcarbonyl, C₄-C₈ cycloalkylcarbonyl, C₂-C₆ alkoxycarbonyl, C₂-C₆ alkylaminocarbonyl, C₃-C₈ dialkylaminocarbonyl or C₄-C₈ cycloalkylaminocarbonyl;
R¹⁷ is H, amino, cyano, hydroxy, C₁-C₆ alkyl, C₁-C₆ haloalkyl, C₃-C₆ alkenyl, C₃-C₆ haloalkenyl, C₃-C₆ alkynyl, C₃-C₆ haloalkynyl, C₃-C₆ cycloalkyl, C₄-C₈ cycloalkylalkyl, C₂-C₆ alkoxyalkyl, C₁-C₆ alkoxy, C₁-C₆ haloalkoxy, C₁-C₆ alkylsulfonyl, C₁-C₆ haloalkylsulfonyl, C₁-C₆ alkylamino, C₁-C₆ haloalkylamino, C₂-C₈ dialkylamino, C₂-C₈ halodialkylamino, C₂-C₆ alkylcarbonyl or C₂-C₆ haloalkylcarbonyl;
R¹⁸ is H, C₁-C₆ alkyl, C₁-C₆ haloalkyl, C₃-C₆ alkenyl, C₃-C₆ alkynyl or C₃-C₆ cycloalkyl; or
R¹⁷ and R¹⁸ are taken together as —(CH₂)₄—, —(CH₂)₅— or —(CH₂)₂O(CH₂)₂—;
R¹⁹ is H, cyano, halogen, C₁-C₄ alkyl, C₁-C₄ haloalkyl, C₂-C₄ alkoxyalkyl, C₂-C₄ alkylcarbonyl, C₂-C₄ alkoxycarbonyl, C₂-C₃ alkylaminocarbonyl or C₃-C₆ dialkylaminocarbonyl;
each R²⁰ is independently H, cyano, C₁-C₆ alkyl, C₁-C₆ haloalkyl, C₃-C₈ cycloalkyl, C₃-C₈ halocycloalkyl, C₁-C₆ alkoxy, C₁-C₆ haloalkoxy, C₁-C₆ alkylamino, C₂-C₈ dialkylamino, C₁-C₆ haloalkylamino or phenyl;
n is 0, 1 or 2; and
s and f are independently 0, 1 or 2 in each instance of S(═O)ₛ(═NR²⁰)_{f};
provided that:
(a) that the sum of s and f is 0, 1 or 2; and
(b) when A is C(═O) or CH(R¹¹) and R¹¹ is hydroxy, then R¹ is bonded through a carbon atom to A.

2. A compound of claim 1 wherein:
E is E-1 or E-2;
X is X¹ or X²;
Y is S;
G is selected from G-12, G-13, G-14, G-15, G-31, G-32 and G-33 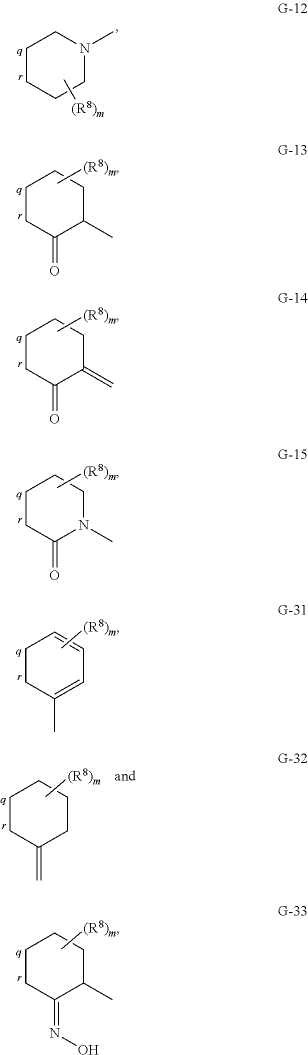
wherein the bond projecting to the right or down is connected to Z in Formula 1;
m is 0, 1 or 2;
Z is NH, CH₂, NHCH₂, CH or NOCH₂, each optionally substituted with up to 1 substituent selected from R^{9a} on a carbon atom and R^{9b} on a nitrogen atom;
Q is selected from Q-45, Q-63, Q-65, Q-70, Q-71, Q-72 and Q-84 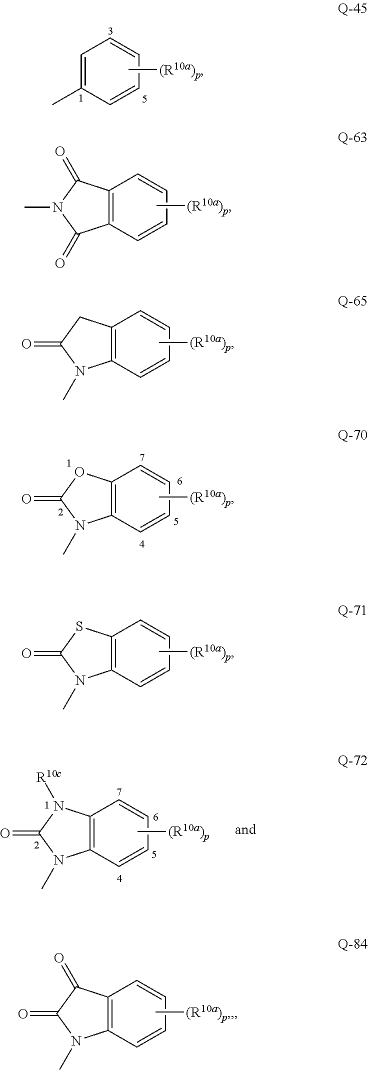
wherein the bond projecting to the left is connected to Z;
p is 0, 1 or 2;
R^{10c} is selected from H and R^{10b};
A is CH(R¹¹) or N(R¹²);
A¹ is O or N(R¹³);
W is O;
R¹ is selected from U-1, U-20 and U-50 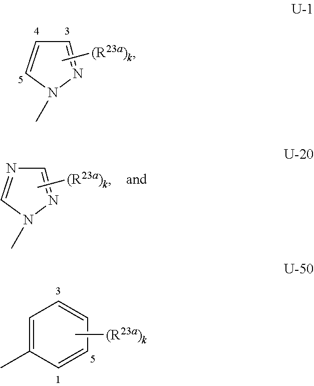
wherein the bond projecting to the left is connected to Formula 1;
k is 0, 1 or 2;
each R^{23a} is independently halogen, C₁-C₃ alkyl, C₁-C₃ haloalkyl or C₂-C₃ alkoxyalkyl;
R² is H, C₁-C₃ alkyl or C₁-C₃ haloalkyl;
R³ is H, C₁-C₃ alkyl or C₁-C₃ haloalkyl;
R⁴ is H or methyl;
R⁵ is H or C₁-C₂ alkyl;
each R^{7a} is independently cyano, halogen, hydroxy, C₁-C₂ alkyl, C₁-C₂ haloalkyl or C₁-C₂ alkoxy;
R⁸ is independently halogen, hydroxy or methyl;
each R^{9a} is halogen, C₁-C₄ alkyl or C₁-C₄ alkoxy;
each R^{9b} is C₁-C₄ alkyl;
each R^{10a} is independently halogen, C₁-C₆ alkyl, C₁-C₆ haloalkyl or C₁-C₆ alkoxy;
R¹¹ is H, halogen, cyano, hydroxy, CH(═O), C₁-C₄ alkyl, C₁-C₄ haloalkyl, C₂-C₅ alkoxycarbonyl or C₁-C₄ alkoxy;
R¹² is H, methyl, CH₃C(═O) or CH₃OC(═O); and
R¹³ is H or methyl.

3. A compound of claim 2 wherein:
E is E-1;
G is selected from G-12, G-13, G-14 and G-15;
m is 0;
Q is Q-45;
A is CH(R¹¹);
R¹ is U-1;
each R^{23a} is independently halogen, methyl or C₁-C₂ haloalkyl;
each R^{9a} is methyl;
each R^{9b} is methyl;
each R^{10a} is independently halogen, C₁-C₂ alkyl, C₁-C₂ haloalkyl or C₁-C₂ alkoxy;
R¹¹ is H; and
n is 0.

4. The compound of claim 3 wherein:
X is X-1;
G is selected from G-13, G-14 and G-15; and
Z is CH₂ or CH.

5. A compound of claim 1 selected from the group consisting of:
6,7-dihydro-2-[1-[2-[5-methyl-3-(trifluoromethyl)-1H-pyrazol-1-yl]acetyl]-4-piperidinyl]-5-(phenylmethyl)thiazol[4,5-c]pyridin-4(5H)-one;
5-[(2,6-difluorophenyl)methyl]-6,7-dihydro-2-[1-[2-[5-methyl-3-(trifluoromethyl)-1H-pyrazol-1-yl]acetyl]-4-piperidinyl]-4(5H)-benzonthiazolone; and
5-[(2,6-difluorophenyl)methyl]-6,7-dihydro-2-[1-[2-[5-methyl-3-(trifluoromethylene)-1H-pyrazol-1-yl]acetyl]-4-piperidinyl]-4(5H)-benzothiazolone.

6. A fungicidal composition comprising (a) a compound of claim 1; and (b) at least one other fungicide.

7. A fungicidal composition comprising (a) a compound of claim 1; and (b) at least one additional component selected from the group consisting of surfactants, solid diluents and liquid diluents.

8. A method for controlling plant diseases caused by fungal plant pathogens comprising applying to the plant or portion thereof, or to the plant seed, a fungicidally effective amount of a compound of claim 1.

9. A method for controlling plant diseases caused by Oomycete fungal plant pathogens comprising applying to the plant or portion thereof, or to the plant seed, a fungicidally effective amount of a compound of claim 1.
